SEQUENCE LISTING (42 sequences)

SEQ ID NO: 1 - DNA, 540 bp, Homo sapiens
gcacccgcccgctcgcccagccccagcacgcagccctgggagtgtgatctgcctcaaacccacagcgcgggtagcaggaggaccttgatggccctggcacagatgaggagaatctctcttttctcctgcttgaaggacagacatgactttggatttccccaggaggagtttggcaaccaggcccaaaaggctgaaaccatccctgtcctccatgagatgatccagcagatcttcgctgccttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattgcggctgtgaggaaatacgcccaaagaatcactgcctatctgaaagagaagaaatacagcccttgtgcctgggaggttgtcagagcagaaaccatgagatctttttctttgtcaacaaacgcgcaagaaagtttaagaagtaaggaatga

SEQ ID NO: 2 - protein, 179 aa, Homo sapiens
APARSPSPSTQPWECDLPQTHSAGSRRTLMALAQMRRISLFSCLKDRHDFGFPQEEFGNQAQKAETIPVLHEMIQQIFAAFSTKDSSAAWDETLLDKFYTELYQQLNDLEACVIQGVGVTETPLMKEDSIAAVRKYAQRITAYLKEKKYSPCAWEVVRAETMRSFSLSTNAQESLRSKE

SEQ ID NO: 3 - DNA, 540 bp, Homo sapiens
gcacccgcccgctcgcccagccccagcacgcagccctgggagtgtgatctgcctcaaacccacagcgcgggtagcaggaggaccttgatgctcctggcacagatgaggagaatctctcttttctcctgcttgaaggacagacatgactttggatttccccaggaggagtttggcaaccaggcccaaaaggctgaaaccatccctgtcctccatgagatgatccagcagatcttcaatctcttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattgcggctgtgaggaaatacgcccaaagaatcactgcctatctgaaagagaagaaatacagcccttgtgcctgggaggttgtcagagcagaaatcatgagatctttttctttgtcaacaaacttgcaagaaagtttaagaagtaaggaatga

SEQ ID NO: 4 - protein, 179 aa, Homo sapiens
APARSPSPSTQPWECDLPQTHSAGSRRTLMLLAQMRRISLFSCLKDRHDFGFPQEEFGNQAQKAETIPVLHEMIQQIFNLFSTKDSSAAWDETLLDKFYTELYQQLNDLEACVIQGVGVTETPLMKEDSIAAVRKYAQRITAYLKEKKYSPCAWEVVRAEIMRSFSLSTNLQESLRSKE

SEQ ID NO: 5 - DNA, 540 bp, Homo sapiens
gcacccgcccgctcgcccagccccagcacgcagccctgggagtgtgatctgcctcaaacccacagcgcgggtagcaggaggaccttgatgctcctggcacagatgaggagaatctctcttttctcctgcttgaaggacagacatgactttggatttccccaggaggagtttggcaaccaggcgcaaaaggctgaaaccatccctgtcctccatgagatgatccagcagatcttcaatctcttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattgcggctgtgaggaaatacgcccaaagaatcactgcctatctgaaagagaagaaatacagcccttgtgcctgggaggttgtcagagcagaaaccatgagatctttttctttgtcaacaaacgcgcaagaaagtttaagaagtaaggaatga

SEQ ID NO: 6 - protein, 179 aa, Homo sapiens
APARSPSPSTQPWECDLPQTHSAGSRRTLMLLAQMRRISLFSCLKDRHDFGFPQEEFGNQAQKAETIPVLHEMIQQIFNLFSTKDSSAAWDETLLDKFYTELYQQLNDLEACVIQGVGVTETPLMKEDSIAAVRKYAQRITAYLKEKKYSPCAWEVVRAETMRSFSLSTNAQESLRSKE

SEQ ID NO: 7 - DNA, 540 bp, Homo sapiens
gcacccgcccgctcgcccagccccagcacgcagccctgggagtgtgatctgcctcaaacccacagcgcgggtagcaggaggaccttgatgctcctggcacagatgaggagaatctctcttttctcctgcttgaaggacagacatgactttggatttccccaggaggagtttggcaaccaggcgcaaaaggctgaaaccatccctgtcctccatgagatgatccagcagatcttcgctgccttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattgcggctgtgaggaaatacgcccaaagaatcactgcctatctgaaagagaagaaatacagcccttgtgcctgggaggttgtcagagcagaaatcatgagatctttttctttgtcaacaaacttgcaagaaagtttaagaagtaaggaatga

SEQ ID NO: 8 - protein, 179 aa, Homo sapiens
APARSPSPSTQPWECDLPQTHSAGSRRTLMLLAQMRRISLFSCLKDRHDFGFPQEEFGNQAQKAETIPVLHEMIQQIFAAFSTKDSSAAWDETLLDKFYTELYQQLNDLEACVIQGVGVTETPLMKEDSIAAVRKYAQRITAYLKEKKYSPCAWEVVRAEIMRSFSLSTNLQESLRSKE

SEQ ID NO: 9 - DNA, 543 bp, Homo sapiens
gcacccgcccgctcgcccagccccagcacgcagccctgggagtgtgacctaccacaaacccacagcctgggtagcaggaggaccttgatgctcctggcgcagatgaggagaatctctcttttctcctgcttgaaggacagacatgactttggatttccccaggaggagtttggcaaccagttccaaaaggctgaaaccatccctgtcctccatgagatgatccagcagatcttcaacctcttcagcacaaaagactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagatctaaagagtaatga

SEQ ID NO: 10 - protein, 179 aa, Homo sapiens
APARSPSPSTQPWECDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 11 - DNA, 501 bp, Homo sapiens
tgtgacctaccacaaacccacagcctgggtagcaggaggaccttgatgctcctggcgcagatgaggagaatctctcttttctcctgcttgaaggacagacatgactttggatttccccaggaggagtttggcaaccagttccaaaaggctgaaaccatccctgtcctccatgagatgatccagcagatcttcaacctcttcagcacaaaagactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagatctaaagagtaatga

SEQ ID NO: 12 - protein, 165 aa, Homo sapiens [sequence omitted]

SEQ ID NO: 13 - DNA, 498 bp, Homo sapiens
tgtgatctgcctcaaacccacagcgcgggtagcaggaggaccttgatggccctggcacagatgaggagaatctctcttttctcctgcttgaaggacagacatgactttggatttccccaggaggagtttggcaaccaggcccaaaaggctgaaaccatccctgtcctccatgagatgatccagcagatcttcgctgccttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattgcggctgtgaggaaatacgcccaaagaatcactgcctatctgaaagagaagaaatacagcccttgtgcctgggaggttgtcagagcagaaaccatgagatctttttctttgtcaacaaacgcgcaagaaagtttaagaagtaaggaatga

SEQ ID NO: 14 - protein, 165 aa, Homo sapiens
CDLPQTHSAGSRRTLMALAQMRRISLFSCLKDRHDFGFPQEEFGNQAQKAETIPVLHEMIQQIFAAFSTKDSSAAWDETLLDKFYTELYQQLNDLEACVIQGVGVTETPLMKEDSIAAVRKYAQRITAYLKEKKYSPCAWEVVRAETMRSFSLSTNAQESLRSKE

SEQ ID NO: 15 - DNA, 498 bp, Homo sapiens
tgtgatctgcctcaaacccacagcgcgggtagcaggaggaccttgatgctcctggcacagatgaggagaatctctcttttctcctgcttgaaggacagacatgactttggatttccccaggaggagtttggcaaccaggcccaaaaggctgaaaccatccctgtcctccatgagatgatccagcagatcttcaatctcttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattgcggctgtgaggaaatacgcccaaagaatcactgcctatctgaaagagaagaaatacagcccttgtgcctgggaggttgtcagagcagaaatcatgagatctttttctttgtcaacaaacttgcaagaaagtttaagaagtaaggaatga

SEQ ID NO: 16 - protein, 165 aa, Homo sapiens
CDLPQTHSAGSRRTLMLLAQMRRISLFSCLKDRHDFGFPQEEFGNQAQKAETIPVLHEMIQQIFNLFSTKDSSAAWDETLLDKFYTELYQQLNDLEACVIQGVGVTETPLMKEDSIAAVRKYAQRITAYLKEKKYSPCAWEVVRAEIMRSFSLSTNLQESLRSKE

SEQ ID NO: 17 - DNA, 498 bp, Homo sapiens
tgtgatctgcctcaaacccacagcgcgggtagcaggaggaccttgatgctcctggcacagatgaggagaatctctcttttctcctgcttgaaggacagacatgactttggatttccccaggaggagtttggcaaccaggcgcaaaaggctgaaaccatccctgtcctccatgagatgatccagcagatcttcaatctcttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattgcggctgtgaggaaatacgcccaaagaatcactgcctatctgaaagagaagaaatacagcccttgtgcctgggaggttgtcagagcagaaaccatgagatctttttctttgtcaacaaacgcgcaagaaagtttaagaagtaaggaatga

SEQ ID NO: 18 - protein, 165 aa, Homo sapiens
CDLPQTHSAGSRRTLMLLAQMRRISLFSCLKDRHDFGFPQEEFGNQAQKAETIPVLHEMIQQIFNLFSTKDSSAAWDETLLDKFYTELYQQLNDLEACVIQGVGVTETPLMKEDSIAAVRKYAQRITAYLKEKKYSPCAWEVVRAETMRSFSLSTNAQESLRSKE

SEQ ID NO: 19 - DNA, 498 bp, Homo sapiens
tgtgatctgcctcaaacccacagcgcgggtagcaggaggaccttgatgctcctggcacagatgaggagaatctctcttttctcctgcttgaaggacagacatgactttggatttccccaggaggagtttggcaaccaggcgcaaaaggctgaaaccatccctgtcctccatgagatgatccagcagatcttcgctgccttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattgcggctgtgaggaaatacgcccaaagaatcactgcctatctgaaagagaagaaatacagcccttgtgcctgggaggttgtcagagcagaaatcatgagatctttttctttgtcaacaaacttgcaagaaagtttaagaagtaaggaatga

SEQ ID NO: 20 - protein, 165 aa, Homo sapiens
CDLPQTHSAGSRRTLMLLAQMRRISLFSCLKDRHDFGFPQEEFGNQAQKAETIPVLHEMIQQIFAAFSTKDSSAAWDETLLDKFYTELYQQLNDLEACVIQGVGVTETPLMKEDSIAAVRKYAQRITAYLKEKKYSPCAWEVVRAEIMRSFSLSTNLQESLRSKE

SEQ ID NO: 21 - protein, 179 aa, Homo sapiens
APARSPSPSTQPWECDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 22 - protein, 165 aa, Homo sapiens
CDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 23 - protein, 179 aa, Homo sapiens
APARSPSPSTQPWECDLPQTHSLGSRRTLMLLAQMRRISLFSCLKDRRDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 24 - protein, 165 aa, Homo sapiens
CDLPQTHSLGSRRTLMLLAQMRRISLFSCLKDRRDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 25 - DNA, 42 bp, Homo sapiens
gcacccgcccgctcgcccagccccagcacgcagccctgggag

SEQ ID NO: 26 - protein, 14 aa, Homo sapiens
APARSPSPSTQPWE

SEQ ID NO: 27 - protein, 179 aa, Homo sapiens
APARSPSPSTQPWECDLPQTHSAGSRRTLMALAQMRKISLFSCLKDRHDFGFPQEEFGNQAQKAETIPVLHEMIQQIFAAFSTKDSSAAWDETLLDKFYTELYQQLNDLEACVIQGVGVTETPLMKEDSIAAVRKYAQRITAYLKEKKYSPCAWEVVRAETMRSFSLSTNAQESLRSKE

SEQ ID NO: 28 - protein, 179 aa, Homo sapiens
APARSPSPSTQPWECDLPQTHSAGSRRTLMLLAQMRKISLFSCLKDRHDFGFPQEEFGNQAQKAETIPVLHEMIQQIFNLFSTKDSSAAWDETLLDKFYTELYQQLNDLEACVIQGVGVTETPLMKEDSIAAVRKYAQRITAYLKEKKYSPCAWEVVRAEIMRSFSLSTNLQESLRSKE

SEQ ID NO: 29 - protein, 179 aa, Homo sapiens
APARSPSPSTQPWECDLPQTHSAGSRRTLMLLAQMRKISLFSCLKDRHDFGFPQEEFGNQAQKAETIPVLHEMIQQIFNLFSTKDSSAAWDETLLDKFYTELYQQLNDLEACVIQGVGVTETPLMKEDSIAAVRKYAQRITAYLKEKKYSPCAWEVVRAETMRSFSLSTNAQESLRSKE

SEQ ID NO: 30 - protein, 179 aa, Homo sapiens
APARSPSPSTQPWECDLPQTHSAGSRRTLMLLAQMRKISLFSCLKDRHDFGFPQEEFGNQAQKAETIPVLHEMIQQIFAAFSTKDSSAAWDETLLDKFYTELYQQLNDLEACVIQGVGVTETPLMKEDSIAAVRKYAQRITAYLKEKKYSPCAWEVVRAEIMRSFSLSTNLQESLRSKE

SEQ ID NO: 31 - protein, 165 aa, Homo sapiens
CDLPQTHSAGSRRTLMALAQMRKISLFSCLKDRHDFGFPQEEFGNQAQKAETIPVLHEMIQQIFAAFSTKDSSAAWDETLLDKFYTELYQQLNDLEACVIQGVGVTETPLMKEDSIAAVRKYAQRITAYLKEKKYSPCAWEVVRAETMRSFSLSTNAQESLRSKE

SEQ ID NO: 32 - protein, 165 aa, Homo sapiens
CDLPQTHSAGSRRTLMLLAQMRKISLFSCLKDRHDFGFPQEEFGNQAQKAETIPVLHEMIQQIFNLFSTKDSSAAWDETLLDKFYTELYQQLNDLEACVIQGVGVTETPLMKEDSIAAVRKYAQRITAYLKEKKYSPCAWEVVRAEIMRSFSLSTNLQESLRSKE

SEQ ID NO: 33 - protein, 165 aa, Homo sapiens
CDLPQTHSAGSRRTLMLLAQMRKISLFSCLKDRHDFGFPQEEFGNQAQKAETIPVLHEMIQQIFNLFSTKDSSAAWDETLLDKFYTELYQQLNDLEACVIQGVGVTETPLMKEDSIAAVRKYAQRITAYLKEKKYSPCAWEVVRAETMRSFSLSTNAQESLRSKE

SEQ ID NO: 34 - protein, 165 aa, Homo sapiens
CDLPQTHSAGSRRTLMLLAQMRKISLFSCLKDRHDFGFPQEEFGNQAQKAETIPVLHEMIQQIFAAFSTKDSSAAWDETLLDKFYTELYQQLNDLEACVIQGVGVTETPLMKEDSIAAVRKYAQRITAYLKEKKYSPCAWEVVRAEIMRSFSLSTNLQESLRSKE

SEQ ID NO: 35 - protein, 179 aa, Homo sapiens
APARSPSPSTQPWECDLPQTHSAGSRRTLMALAQMRRISLFSCLKDRRDFGFPQEEFGNQAQKAETIPVLHEMIQQIFAAFSTKDSSAAWDETLLDKFYTELYQQLNDLEACVIQGVGVTETPLMKEDSIAAVRKYAQRITAYLKEKKYSPCAWEVVRAETMRSFSLSTNAQESLRSKE

SEQ ID NO: 36 - protein, 179 aa, Homo sapiens
APARSPSPSTQPWECDLPQTHSAGSRRTLMLLAQMRRISLFSCLKDRRDFGFPQEEFGNQAQKAETIPVLHEMIQQIFNLFSTKDSSAAWDETLLDKFYTELYQQLNDLEACVIQGVGVTETPLMKEDSIAAVRKYAQRITAYLKEKKYSPCAWEVVRAEIMRSFSLSTNLQESLRSKE

SEQ ID NO: 37 - protein, 179 aa, Homo sapiens
APARSPSPSTQPWECDLPQTHSAGSRRTLMLLAQMRRISLFSCLKDRRDFGFPQEEFGNQAQKAETIPVLHEMIQQIFNLFSTKDSSAAWDETLLDKFYTELYQQLNDLEACVIQGVGVTETPLMKEDSIAAVRKYAQRITAYLKEKKYSPCAWEVVRAETMRSFSLSTNAQESLRSKE

SEQ ID NO: 38 - protein, 179 aa, Homo sapiens
APARSPSPSTQPWECDLPQTHSAGSRRTLMLLAQMRRISLFSCLKDRRDFGFPQEEFGNQAQKAETIPVLHEMIQQIFAAFSTKDSSAAWDETLLDKFYTELYQQLNDLEACVIQGVGVTETPLMKEDSIAAVRKYAQRITAYLKEKKYSPCAWEVVRAEIMRSFSLSTNLQESLRSKE

SEQ ID NO: 39 - protein, 165 aa, Homo sapiens
CDLPQTHSAGSRRTLMALAQMRRISLFSCLKDRRDFGFPQEEFGNQAQKAETIPVLHEMIQQIFAAFSTKDSSAAWDETLLDKFYTELYQQLNDLEACVIQGVGVTETPLMKEDSIAAVRKYAQRITAYLKEKKYSPCAWEVVRAETMRSFSLSTNAQESLRSKE

SEQ ID NO: 40 - protein, 165 aa, Homo sapiens
CDLPQTHSAGSRRTLMLLAQMRRISLFSCLKDRRDFGFPQEEFGNQAQKAETIPVLHEMIQQIFNLFSTKDSSAAWDETLLDKFYTELYQQLNDLEACVIQGVGVTETPLMKEDSIAAVRKYAQRITAYLKEKKYSPCAWEVVRAEIMRSFSLSTNLQESLRSKE

SEQ ID NO: 41 - protein, 165 aa, Homo sapiens
CDLPQTHSAGSRRTLMLLAQMRRISLFSCLKDRRDFGFPQEEFGNQAQKAETIPVLHEMIQQIFNLFSTKDSSAAWDETLLDKFYTELYQQLNDLEACVIQGVGVTETPLMKEDSIAAVRKYAQRITAYLKEKKYSPCAWEVVRAETMRSFSLSTNAQESLRSKE

SEQ ID NO: 42 - protein, 165 aa, Homo sapiens
CDLPQTHSAGSRRTLMLLAQMRRISLFSCLKDRRDFGFPQEEFGNQAQKAETIPVLHEMIQQIFAAFSTKDSSAAWDETLLDKFYTELYQQLNDLEACVIQGVGVTETPLMKEDSIAAVRKYAQRITAYLKEKKYSPCAWEVVRAEIMRSFSLSTNLQESLRSKE

(19) United States
(12) Patent Application Publication
(10) Pub. No.: US 2023/0127506 A1
(43) Pub. Date: Apr. 27, 2023

(54) MODIFIED INTERFERON-ALPHA-2 HAVING REDUCED IMMUNOGENICITY

(71) Applicants: EPIVAX, INC., Providence, RI (US); UNIVERSIDAD NACIONAL DEL LITORAL, Santa Fe (AR); CONSEJO NACIONAL DE INVESTIGACIONES CIENTÍFICAS Y TÉCNICAS, Caba (AR)

(72) Inventors: Eduardo Federico Mufarrege, Santa Fe (AR); Sofía Inés Giorgetti, Santa Fe (AR); Marina Etcheverrigaray, Santa Fe (AR); Anne Searls De Groot, Providence, RI (US); William D. Martin, Providence, RI (US)

(21) Appl. No.: 17/783,948

(22) PCT Filed: Dec. 16, 2020
(86) PCT No.: PCT/US20/65246

§ 371 Date: Jun. 9, 2022

(30) Foreign Application Priority Data
Dec. 17, 2019 (AR) 20190103715

(51) Int. Cl. C07K 14/56

(52) U.S. Cl. CPC C07K 14/56; A61K 38/00

(57) ABSTRACT

The present disclosure is directed to compositions comprising modified interferon-α2 polypeptides having interferon-α2 activity and reduced immunogenicity. In aspects, said modified interferon-α2 polypeptides are hyperglycosylated, such as by addition of a GM-CSF-derived peptide sequence with multiple O-glycosylation sites. Furthermore, the present disclosure provides compositions comprising a nucleic acid molecule encoding said modified interferon-α2. The present disclosure also provides compositions comprising a recombinant protein expression cell line comprising said nucleic acid molecule encoding said modified interferon-α2; wherein said recombinant protein expression cell comprises a plasmid or vector containing said nucleic acid molecule. Also disclosed are pharmaceutical compositions comprising a modified interferon-α2 having interferon-α2 activity with reduced immunogenicity, as well as methods of use of said pharmaceutical formulations for treatment of medical conditions in a subject.

### CROSS-REFERENCE TO RELATED APPLICATION

This application depends from and claims priority to Argentina Provisional Application No: 20190103715, titled “Hyperglycosylated Interferon with Reduced Immunogenicity and filed Dec. 17, 2019, the entire contents of which is incorporated herein by reference.

### REFERENCE TO SEQUENCE LISTING SUBMITTED ELECTRONICALLY

The instant application contains a Sequence Listing which has been submitted electronically in ASCII format and is hereby incorporated by reference in its entirety. Said ASCII copy, created Dec. 11, 2020, is named “EPV0027WO Sequence Patent-In_ST25” and is 54 KB bytes in size.

### TECHNICAL FIELD

The present disclosure generally relates to the development of therapeutic molecules of pharmaceutical interest for application to humans. More particularly, the present disclosure relates to modified IFNα-2 polypeptides (including modified IFNα-2a, IFNα-2b, and IFNα-2c polypeptides), as well as to compounds and compositions. These modified IFNα-2 polypeptides display proven antiviral biological activity, improved pharmacokinetic parameters with respect to commercial cytokine, and reduced immunogenicity. These modified IFNα-2 polypeptides, as well as related compounds and compositions, can be used for human therapy and treatments, including antiviral therapy.

### BACKGROUND

Recombinant proteins for therapeutic use are part of routine medical practice and are used for the treatment of a wide variety of diseases. They account for more than 20% of the pharmaceutical market and their growth rate has doubled that of drugs based on small molecules. Therapeutic protein-based treatments typically have high rates of efficacy with limited adverse effects. Indeed, the use of biotherapeutics has provided possibilities for medical intervention that would not have been possible through the application of other types of drugs in the treatment of numerous human diseases, from microbial infections to various types of cancers, arthritis and autoimmune diseases.

However, the clinical application of therapeutic proteins entails overcoming a number of challenges, both from an operational and manufacturing point of view, as well as from the clinical limitations of the product. For example, issues with the administration of proteins as therapeutics include poor solubility, poor stability, short circulation half-life, and issues with retaining biological function. Additionally, achieving a readily administrable therapeutic may be difficult, as production of a composition containing pure protein with a high yield may entail many challenges. Thus, the efforts made to develop biotherapeutics capable of generating an effective and sustained biological response over time are not surprising.

Although in most cases, these proteins (cytokines, growth factors and monoclonal antibodies, among others) constitute molecules almost identical to those produced by the human body, numerous cases of immunological responses developed as a result of the administration of these drugs have been reported. Antibodies developed against these drugs (ADAs) can affect protein activity and produce effects of varying complexity and severity, depending on factors such as title, duration in circulation and its neutralizing activity. The most common consequences involve decreased treatment efficacy and hypersensitivity reactions, although they can also trigger anaphylaxis and autoimmune diseases. The prevalence of developed antibodies ranges from less than 1% for drugs such as the tissue plasminogen activator (Activase) to 70% for drugs such as OKT3, an IgG₂a monoclonal antibody.

The generation of neutralizing antibodies in response to administration of therapeutic proteins can occur as a result of various factors, which can be grouped into two broad categories: extrinsic factors, such as route of administration, dose, formulation, presence of aggregates and/or contaminants, and/or the presence and/or type of glycosylations; and intrinsic factors, including the presence of immunogenic epitopes in the protein.

The extrinsic factors are fundamentally related to the design and quality of the production process. In this sense, contamination of the product with pro-inflammatory agents or mutagenic nonspecific compounds, such as LPS (bacterial lipopolysaccharide), or the generation of aggregates in the product, can generate a critical signal for induction of an immune response. In addition, the denaturation of the therapeutic, which may take place during formulation, may lead to products with greater immunogenicity than their intact counterparts, due to the presence of new epitopes capable of being recognized by B-lymphocytes, leading to the stimulation of an immune response with the development of ADAs. In most cases, these factors have been successfully circumvented by the development of careful production processes, both in the stages prior to the production of the drug and in the final stages of purification of the product. In addition, good results have been achieved by incorporating excipients that stabilize the biotherapeutic.

However, intrinsic factors are a real challenge, as activation of B-lymphocytes contributes to the development of antibodies, even in cases where the therapeutic is virtually identical to autologous protein. Activation of B-lymphocytes may or may not be mediated by the collaboration of T-cells, resulting in T-dependent or T-independent responses, respectively. T-independent responses develop as a result of the activation of a particular group of B-lymphocytes, which are stimulated by certain structural characteristics of some molecules, such as polymeric repetitions. Antibodies developed as a result of this T-independent activation are primarily of the low affinity IgM type.

In contrast, T-cell-dependent activation is primarily associated with the primary protein sequence. In T-cell-dependent activation, when the molecule is endocytosed, processed and the resulting peptides are presented on the surface of antigen-presenting cells (dendritic cells, Macrophages or B lymphocytes) in the context of Class II Major Histocompatibility Complex (MHC) molecules, some sequences may be recognized by T cells “helper” (Tₕ) (via their receptor on the cell surface, called TCR (T Cell Receptor)). These specific lymphocytes, once activated, will trigger an immune response that will lead to B lymphocyte activation and consequent ADA production. In T-cell-dependent responses, the antibodies developed are of the IgG type, have a higher affinity and generation is more prolonged and sustained over time than those generated without the participation of T cells. Currently, there is a wide multiplicity of methodologies that allow for evaluation of the potential immunogenicity of therapeutic proteins, including computational or in silico immunogenicity prediction techniques, strategies for growing in vitro and ex vivo cells, and the use of animal models. All of them are based on the premise that immune responses to proteins of most interest therapeutic use are dependent on T cells.

Activation of T-cell-dependent B lymphocytes begins with the interaction of a group of B-lymphocytes with certain protein epitopes through their antigenic receptors (IgM/IgD) on the cell surface, constituting the first sign of activation of B-lymphocytes. This signal promotes the internalization of the protein that will then be processed into small peptide epitopes, which will eventually be exposed within the “groove” of Class II MHC molecules on the surface of B-lymphocytes. B cells also co-express the CD40 molecule on its surface. When Tₕ (helper T) lymphocytes interact through their TCR and the ligand of the molecule CD40 (CD154) with the complex epitope-MHC class II and with CD40 (on the surface of B lymphocytes), they trigger the second activation signal. This signal eventually activates B-lymphocytes and T cells produce, among others, cytokine IL-4 (in a response of Tₕ lymphocytes type 2) or interferon γ (Tₕ lymphocytes type 1) causing the maturation of the immune response. It should be noted that without the participation of T cells, which provide the second signal, B-lymphocytes suffer a scheduled cell death (apoptosis). For this reason, attenuation of an immune response mediated by T cells has become the focus of attention on the process known as “de-immunization” of recombinant proteins for therapeutic purposes.

In particular, in the case of treatments with IFNα or IFN-β, despite being autologous cytokines, some patients have observed a break in immune tolerance to their own antigens, resulting in the production of anti-IFN antibodies. These antibodies can bind to the IFN molecule without producing virtually any effect, or may alter the pharmacokinetics of the cytokine, causing the neutralization of its activity by blocking the binding domains to specific receptors on the surface of target cells. Indeed, numerous clinical studies have shown the development of anti-IFN-α antibodies in patients with chronic hepatitis C or neoplastic diseases treated with IFNα-2a or IFNα-2b.

Another major limitation associated with the use of IFNα-2 (including IFNα-2b) as a biotherapeutic is its short half-life in circulation, which leads to the need for prolonged treatments, resulting in the possible occurrence of the aforementioned adverse effects. In this sense, the PEGylation of the molecule has allowed to increase its half-life in plasma, allowing a weekly dosage and with improved efficiency compared to the native molecule. PEGylation is often incorporated as a strategy that reduces the immunogenicity of recombinant proteins, because it exerts an erric impairment that often reduces antigenic presentation. However, there is data showing that 8% of patients with chronic hepatitis C who do not respond to PEGylated IFNα-2 and ribavirin therapy had anti-IFN neutralizing antibodies, while none of the patients who eliminated HCV virus after treatment with IFN showed detectable levels of these antibodies.

Some strategies for improving plasma half-life target renal clearance, as it is a predominant fast elimination route. The glomerular barrier filters protein according to their charge and size, so the starting point for decreasing plasma clearance has been altering their hydrodynamic volume. As such, with the aim of improving the pharmacokinetics of different biotherapeutics, in recent years N- and O-glycosylation engineering strategies have been implemented, which allow for generation of glycoproteins with very low glomerular filtration rates. This result is due to the greater hydrodynamic radius that is conferred by the presence of glycans, as well as the negative charge of the terminal sialic acids of the glycans, which undergo a repulsive interaction with the negatively charged glycosaminoglycans of the glomerular pores.

However, despite the favorable results that have been obtained through the implementation of this strategy in terms of increased half-life, the introduction of a set of mutations in the coding sequence of the protein, in order to generate the consensus sites of N-glycosylation or large regions rich in Ser/Tre required for O-glycosylation (which lack an understood consensus site), usually have a negative impact on the biological activity of the therapeutic. An example of this is the development of a hyperglycosylated version of the wild type IFNα-2b by incorporating 4 N-glycosylation sites, which achieved a 25-fold increase in the half-life of modified cytokine (IFN-2b-4N) as compared to the wild type protein. However, the in vitro biological activity of IFN-2b-4N was less than 80% compared to the wild type protein.

Thus, there is a need in the art for interferon-derived protein therapeutics that not only have improved pharmacokinetic parameters and/or reduced immunogenicity, and thus better safety among patient populations; but that also retain their biological activity and therapeutic efficacy, such as their antiviral activity, and that are easy to produce and purify.

### SUMMARY

Accordingly, the present disclosure provides modified IFNα-2 polypeptides and related compositions displaying proven antiviral biological activity and having reduced immunogenicity and improved pharmacokinetic parameters with respect to wild-type IFNα-2 and available commercial cytokine. The modified IFNα-2 polypeptides find use as a therapeutic in human subjects for a variety of reasons, such as better safety among patient populations, ease in production and purification, reduced immunogenicity, improved pharmacokinetic profile, high relative antiviral activity, and low antiproliferative biological activity.

In aspects, the present disclosure provides a modified interferon-α2 polypeptide with reduced immunogenicity. In aspects, said modified interferon-α2 is a modified interferon-α2b polypeptide, interferon-α2a polypeptide, or interferon-α2c polypeptide. In aspects, said modified interferon-α2 polypeptide comprises the substitution of one or more amino acids occupying positions selected from the group consisting of the following positions in the natural human interferon-α2: 9, 17, 47, 65, 66, 117, 123, 128, 147, and 157, where such substitution includes the change of the amino acid from that position to an amino acid selected from the group consisting of: alanine, glycine, or threonine. In aspects, said modified interferon-α2 polypeptide comprises the substitution of one or more amino acids occupying positions selected from the group consisting of the following positions in the natural human interferon-α2: 9, 17, 47, 65, 66, 117, 123, 128, 147, and 157, where such mutations reduce the immunogenicity of the modified interferon-α2 as compared to the natural human interferon-α2. In aspects of the above-described polypeptides, the modified interferon-α2 polypeptides may be isolated, synthetic, or recombinant.

In aspects, a modified interferon-α2 also comprises the addition of amino acids containing one or more sites of N or O glycosylation. In aspects, a modified interferon-α2 also comprises the addition of amino acids containing one or more sites of N or O glycosylation, wherein these added amino acids comprise a sequence with at least 80%, 90, or 95% homology to APARSPSPSTQPWE or a fragment thereof. In aspects, it includes the addition of the amino acid sequence APARSPSPSTQPWE (SEQ ID NO: 26) or a fragment thereof. In aspects, said fragment of APARSPSPSTQPWE is at least 7, at least 8, at least 9 and/or at least 10 amino acids in length. Such amino acid additions may be added to the N-terminus and/or C-terminus of the instantly-disclosed modified interferon-α2 polypeptides. In aspects of the above-described polypeptides, the modified interferon-α2 polypeptides may be isolated, synthetic, or recombinant.

In aspects, the present disclosure is directed to a modified interferon-α2b polypeptide having interferon-α2 activity, the polypeptide comprising an amino acid sequence comprising at least 60, 70, 80, 90, or 95% homology to SEQ ID NO: 12 and further comprising at least five amino acid substitutions in any of the positions selected from the set comprised of: 9, 17, 47, 65, 66, 117, 123, 128, 147, and 157; wherein said substitution comprises the change of the amino acid of said position to alanine, glycine, or threonine. In aspects, said substitutions comprise the mutations: L9A, F47A, L117A, F123A, and L128A. In aspects, said substitutions comprise the mutations: L9A, F47A, L117A, F123A, L128A, I147T and L157A. In aspects, said substitutions comprise the mutations: L9A, F47A, N65A, L66A, L117A, F123A, and L128A. In aspects, said substitutions comprise the mutations: L9A, L17A, F47A, N65A, L66A, L117A, F123A, L128A, 1147T and L157A.

In aspects, the present disclosure is directed to a modified GMOP-interferon-α2b polypeptide having interferon-α2 activity, the polypeptide comprising an amino acid sequence comprising at least 60, 70, 80, 90, or 95% homology to SEQ ID NO: 10 and further comprising at least five amino acid substitutions in any of the positions selected from the set comprised of: 23, 31, 61, 79, 80, 131, 137, 142, 161, and 171; wherein said substitution comprises the change of the amino acid of said position to alanine, glycine, or threonine. In aspects, said substitutions comprise the mutations: L23A, F61A, L131A, F137A, and L142A. In aspects, said substitutions comprise the mutations: L23A, F61A, L131A, F137A, L142A, I161T, and L171A. In aspects, said substitutions comprise the mutations: L23A, F61A, N79A, L80A L131A, F137A, and L142A. In aspects, said substitutions comprise the mutations: L23A, L31A, F61A, N79A, L80A L131A, F137A, L142A, I161T, and L171A.

In aspects, the present disclosure is directed to a modified interferon-α2a polypeptide having interferon-α2 activity, the polypeptide comprising an amino acid sequence comprising at least 60, 70, 80, 90, or 95% homology to SEQ ID NO: 22 and further comprising at least five amino acid substitutions in any of the positions selected from the set comprised of: 9, 17, 47, 65, 66, 117, 123, 128, 147, and 157; wherein said substitution comprises the change of the amino acid of said position to alanine, glycine, or threonine. In aspects, said substitutions comprise the mutations: L9A, F47A, L117A, F123A, and L128A. In aspects, said substitutions comprise the mutations: L9A, F47A, L117A, F123A, L128A, I147T and L157A. In aspects, said substitutions comprise the mutations: L9A, F47A, N65A, L66A, L117A, F123A, and L128A. In aspects, said substitutions comprise the mutations: L9A, L17A, F47A, N65A, L66A, L117A, F123A, L128A, 1147T and L157A.

In aspects, the present disclosure is directed to a modified GMOP-interferon-α2a polypeptide having interferon-α2 activity, the polypeptide comprising an amino acid sequence comprising at least 60, 70, 80, 90, or 95% homology to SEQ ID NO: 21 and further comprising at least five amino acid substitutions in any of the positions selected from the set comprised of: 23, 31, 61, 79, 80, 131, 137, 142, 161, and 17; wherein said substitution comprises the change of the amino acid of said position to alanine, glycine, or threonine. In aspects, said substitutions comprise the mutations: L23A. F61A. L131A, F137A. and L142A. In aspects, said substitutions comprise the mutations: L23A, F61A, L131A, F137A, L142A, I161T, and L171A. In aspects, said substitutions comprise the mutations: L23A, F61A, N79A, L80A L131A, F137A, and L142A. In aspects, said substitutions comprise the mutations: L23A, L31A, F61A, N79A, L80A L131A, F137A, L142A, I161T, and L171A.

In aspects, the present disclosure is directed to a modified interferon-α2c polypeptide having interferon-α2 activity, the polypeptide comprising an amino acid sequence comprising at least 60, 70, 80, 90, or 95% homology to SEQ ID NO: 24 and further comprising at least five amino acid substitutions in any of the positions selected from the set comprised of: 9, 17, 47, 65, 66, 117, 123, 128, 147, and 157; wherein said substitution comprises the change of the amino acid of said position to alanine, glycine, or threonine. In aspects, said substitutions comprise the mutations: L9A, F47A, L117A, F123A, and L128A. In aspects, said substitutions comprise the mutations: L9A, F47A, L117A, F123A, L128A, I147T and L157A. In aspects, said substitutions comprise the mutations: L9A, F47A, N65A, L66A, L117A, F123A, and L128A. In aspects, said substitutions comprise the mutations: L9A, L17A, F47A, N65A, L66A, L117A, F123A, L128A, 1147T and L157A.

In aspects, the present disclosure is directed to a modified GMOP-interferon-α2c polypeptide having interferon-α2 activity, the polypeptide comprising an amino acid sequence comprising at least 60, 70, 80, 90, or 95% homology to SEQ ID NO: 23 and further comprising at least five amino acid substitutions in any of the positions selected from the set comprised of: 23, 31, 61, 79, 80, 131, 137, 142, 161, and 17; wherein said substitution comprises the change of the amino acid of said position to alanine, glycine, or threonine. In aspects, said substitutions comprise the mutations: L23A, F61A, L131A, F137A, and L142A. In aspects, said substitutions comprise the mutations: L23A, F61A, L131A, F137A, L142A, I161T, and L171A. In aspects, said substitutions comprise the mutations: L23A, F61A, N79A, L80A L131A, F137A, and L142A. In aspects, said substitutions comprise the mutations: L23A, L31A, F61A, N79A, L80A L131A, F137A, L142A, I161T, and L171A.

In aspects, a modified interferon-α2 polypeptide is selected from the group consisting of: SEQ ID NO: 14, SEQ ID NO: 16, SEQ ID NO: 18, and SEQ ID NO: 20. In aspects, a modified interferon-α2 polypeptide is selected from the group consisting of: SEQ ID NO: 2, SEQ ID NO: 4, SEQ ID NO: 6, and SEQ ID NO: 8. In aspects, said modified interferon-α2 is selected from the group consisting of: SEQ ID NO: 4 and SEQ ID NO: 6.

In aspects, the instantly-disclosed modified interferon-α2 polypeptide has antiviral activity that is comparable to the antiviral activity of the natural human interferon-α2. In aspects, said modified interferon-α2 has a Relative Antiviral Activity of between 10 and 90%, as compared with the antiviral activity of the natural human interferon-α2.

In aspects, the present disclosure is directed to a polynucleotide (e.g., DNA or RNA) encoding one or more of the modified polypeptides of the present disclosure. In aspects of the instantly-disclosed polynucleotides, the polynucleotides may be isolated, synthetic, or recombinant. In aspects, an expression cassette, plasmid, expression vector, and recombinant virus comprising such a polynucleotide is provided. In aspects, a microorganism or cell comprising an expression cassette, plasmid, vector, or recombinant virus of the present disclosure is provided. In aspects, the present disclosure is directed to a characterized cell line comprising the nucleic acid that encodes for one or more modified interferon-α2 polypeptides of the invention, which also presents reduced immunogenicity. In aspects, this cell line is suitable for the production of modified interferon-α2 with reduced immunogenicity. Preferably, this cell line is selected from the group consisting of: CHO-K1, HEK293, NS0, BHK, Sp2/0, CAP, and CAP/T.

In aspects, the instant disclosure is directed to a pharmaceutical composition, the pharmaceutical composition comprising one or more modified IFN-α2 polypeptides, nucleic acids, cells, and/or vectors as disclosed herein and optionally a pharmaceutically acceptable excipient and/or carrier. In aspects, the instantly-disclosed pharmaceutical compositions comprising at least one or more modified IFN-α2 polypeptides, nucleic acids, cells, and/or vectors may be used for treatment of diseases, such as melanomas (including malignant melanoma), chronic hepatitis C (including in patients with compensated liver disease), acute and chronic hepatitis B, acute and chronic non-A, non-B hepatitis, Kaposi's sarcoma (including AIDS-related Kaposi's sarcoma), multiple sclerosis, genital warts, leukemia (including Hairy cell leukemia), lymphomas (including follicular lymphoma), condylomata acumiate, viral infections (including SARS-CoV-2 infection ZIKV infection, CHIKV infection, or influenza A infection), among others.

In aspects, the present disclosure is direct to methods of preventing or treating one or more medical conditions in a subject comprising administering one or more modified interferon-α2 compounds or compositions of the present disclosure, and preventing or treating the medical condition in a subject by said step of administering said one or more modified interferon-α2 compounds or compositions of the present disclosure. The medical condition can be, for example against melanomas, melanomas (including malignant melanoma), chronic hepatitis C (including in patients with compensated liver disease), acute and chronic hepatitis B, acute and chronic non-A, non-B hepatitis, Kaposi's sarcoma (including AIDS-related Kaposi's sarcoma), multiple sclerosis, genital warts, leukemia (including Hairy cell leukemia), lymphomas (including follicular lymphoma), condylomata acumiate, and other viral infections (including SARS-CoV-2 infection ZIKV infection, CHIKV infection, or influenza A infection).

In aspects, the present disclosure provides the use of one or more modified interferon-α2 compounds or compositions of the present disclosure for manufacturing a medicament for the treatment of melanomas (including malignant melanoma), chronic hepatitis C (including in patients with compensated liver disease), acute and chronic hepatitis B, acute and chronic non-A, non-B hepatitis, Kaposi's sarcoma (including AIDS-related Kaposi's sarcoma), multiple sclerosis, genital warts, leukemia (including Hairy cell leukemia), lymphomas (including follicular lymphoma), condylomata acumiate, viral infections (including SARS-CoV-2 infection ZIKV infection, CHIKV infection, or influenza A infection).

### BRIEF DESCRIPTION OF THE DRAWINGS

The present disclosure may be better understood with reference to the following figures.

FIGS. 1A-B depict in silico immunogenicity analysis of GMOP-IFNα-2b. EpiMatrix-predicted 9-mer hits for 8 prevalent HLA class II alleles are aligned along the GMOP-IFN2b sequence. Peptides scoring above 1.64 on the EpiMatrix “Z” scale (top 5%) are considered to be potential epitopes (gray bars). Peptides scoring above 2.32 on the scale (top 1%) are extremely likely to bind MHC (black bars). Clusters identified by EpiMatrix with the respective scores indicated above are framed. Published epitopes (bars below map) determined by experimental methods overlapped with those defined here. FIG. 1A shows predicted MHC Class II binding clusters of GMOP-IFN as predicted by EpiMatrix. A total of six binding clusters were predicted. FIG. 1B shows the impact of 10 selected mutations on the overall potential immunogenicity of GMOP-IFN.

FIG. 2 shows the EpiMatrix MHC binding cluster immunogenicity scale. GMOP-IFN-2b and its deimmunized variants (GMOP-IFN-VAR1, GMOP-IFN-VAR2, GMOP-IFN-VAR3, and GMOP-IFN-VAR4) are mapped onto a cluster immunogenicity scale according to their individual EpiMatrix scores. The EpiMatrix cluster immunogenicity score represents the deviation in putative epitope content from baseline expectation based on a random peptide standard. MHC binding clusters scoring above +10 are considered to be potentially immunogenic, while MHC binding clusters scoring lower are considered to have less potential to be immunogenic. Some positive control peptides and proteins are also arranged by EpiMatrix score of immunogenicity, from highest (+80) to lowest (−50).

FIG. 3 depicts a purity evaluation of different modified GMOP-IFNα-2b polypeptides by denaturing SDS-PAGE gel following one-step immunoaffinity chromatography. Purity levels above 94% were achieved. Lane 1 contains the protein molecular weight marker. Lane 2 contains non-glycosylated IFN-α2b. Lane 3 contains wild type IFN-α2b. Lane 4 contains GMOP-IFN-α2b. Lane 5 contains GMOP-IFN-α2b-VAR1. Lane 6 contains GMOP-IFN-α2b-VAR2. Lane 7 contains GMOP-IFN-α2b-VAR3. Lane 8 contains GMOP-IFN-α2b-VAR4.

FIG. 4 depicts an isoelectric focusing assay. The charge-based heterogeneity of the modified GMOP-IFN variants was analyzed by IEF followed by Coomasie blue staining. Differently sialylated forms were distinguished for each protein variant, revealing 7 isoforms for GMOP-IFN and 11 electrophoretic bands for both GMOP-IFN-VAR2 and 3. GMOP-IFN deimmunized variants exhibited a higher content of glycan structures bound to the O-glycosylation moieties. Lane 1 contains wild type IFN-α2b. Lane 2 contains GMOP-IFN-α2B. Lane 3 contains GMOP-IFN-α2B-VAR2. Lane 4 contains GMOP-IFN-α2B-VAR3. The content of sialic acid increases from the top portion of the gel to the bottom portion of the gel.

FIG. 5 depicts a sandwich ELISA which measured IFN-γ secretion by T-cells after incubation with IFN-pulsed dendritic cells. The data was obtained from 20 donors. A Stimulation Index (SI) was defined as a ratio of the cytokine concentration from protein challenged samples divided by cytokine concentration from excipient treated samples. Differences between treatments were evaluated through a one-way analysis of variance (ANOVA). Differences were considered statistically significant when p<0.05. A post-hoc Tukey's multiple comparison test was then applied. Modified GMOP-IFN-alpha molecules exhibited a reduced immunogenicity in comparison with the original molecule.

FIG. 6 depicts an HLA-DR antibody blocking assay to study the HLA restriction of IFN-derived peptide presentation by DC. A successive decrease in IFN-γ Stimulation Index (SI) was observed when two different blocking Ab concentrations were evaluated. SI were normalized to the untreated control (excipients). IFN-derived peptides are presented in the context of HLA-DR molecules.

FIG. 7 is a graph that depicts the IFN-α2 pharmacokinetic plasma profiles in Wistar rats at different post-injection times after subcutaneous injection. Plasma protein concentration was plotted versus time. Data points represent the average±SEM of four animals in each group.

FIG. 8 shows a Sandwich ELISA test performed with the supernatants of the production lines of each variant of GMOP-IFN-α2b. The supernatants corresponding to GMOP-IFN-α2b-VAR1 and GMOP-IFN-α2b-VAR4 were pure, while those corresponding to GMOP-IFN-α2b-VAR2 and GMOP-IFN-α2b-VAR3 were diluted 1/20 in order to perform a preliminary quantification of each protein. All the supernatants showed the presence of the cytokine of interest.

FIG. 9 depicts data from a preliminary antiviral activity test performed on cell line culture supernatants producing the different de-immunized variants of GMOP-IFN-α2b. The absorbance data were plotted as a function of the corresponding activity values of IFN-α2b (standard) and of the dilutions of the samples on a logarithmic scale and the biological activity values (AB) were calculated for each of the molecules by comparison. All the supernatants showed antiviral activity at different magnitudes.

FIG. 10 depicts an antiviral biological assessment test of purified GMOP-IFN-2b and two purified de-immunized variants of GMOP-IFN2b: GMOP-IFN-2b-VAR1 and GMOP-IFN-2b-VAR4. The quantification of the specific activity of each molecule was determined from comparison with an international standard (NIBSC). The percentage relative antiviral activity value was calculated.

FIG. 11 depicts an antiviral biological assessment test of two purified deimmunized variants of GMOP-IFN-2b: GMOP-IFN-2b-VAR2 and GMOP-IFN-2b-VAR3. The quantification of the specific activity of each molecule was determined from comparison with an international standard (NIBSC). The percentage relative antiviral activity value was calculated.

### DETAILED DESCRIPTION

### General

The following description of particular aspect(s) is merely exemplary in nature and is in no way intended to limit the scope of the present disclosure, its application, or uses, which may, of course, vary. The present disclosure is described with relation to the non-limiting definitions and terminology included herein. These definitions and terminology are not designed to function as a limitation on the scope or practice of the present disclosure but are presented for illustrative and descriptive purposes only. While the processes and compositions are described as using specific a specific order of individual steps or specific materials, it is appreciated that steps or materials may be interchangeable such that the description of the present disclosure may include multiple steps or parts arranged in many ways as is readily appreciated by one of skill in the art.

Reference will now be made in detail to various embodiments of the instantly-disclosed modified IFNα-2 polypeptides (including modified IFNα-2b, IFN-α2a, and IFN-α2c polypeptides) with proven antiviral biological activity, improved pharmacokinetic parameters with respect to commercial cytokine, and reduced immunogenicity, nucleic acids that encode such modified IFNα-2 polypeptides, expression cassettes, plasmids, expression vectors, recombinant viruses, or cells comprising such nucleic acids, and modified IFNα-2 polypeptides pharmaceutical compositions and formulations. As described, these various compounds and compositions find use in treating various virus infections, including chronic hepatitis B, chronic hepatitis C, and condylomata acuminate, as well as hairy cell leukemia, malignant melanoma, AIDS-related Kaposi's sarcoma, follicular non-Hodgkin's lymphoma.

Although any methods and materials similar or equivalent to those described herein can be used in the practice or testing of the present disclosure, the preferred methods and materials are described. Other features, objects, and advantages of the present disclosure will be apparent from the description and the Claims. In the Specification and the appended Claims, the singular forms include plural referents unless the context clearly dictates otherwise. All references cited herein are incorporated herein by reference in their entirety and for all purposes to the same extent as if each individual publication, patent, or patent application was specifically and individually indicated to be incorporated by reference in its entirety for all purposes.

### Definitions

To further facilitate an understanding of the present disclosure, a number of terms and phrases are defined below. Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

The terminology used herein is for describing particular embodiments/aspects only and is not intended to be limiting.

As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

As used herein, the term “biological sample” as refers to any sample of tissue, cells, or secretions from an organism.

As used herein, the term “medical condition” includes, but is not limited to, any condition or disease manifested as one or more physical and/or psychological symptoms for which treatment and/or prevention is desirable, and includes previously and newly identified diseases and other disorders.

As used herein, the term “immune response” refers to the concerted action of lymphocytes, antigen presenting cells, phagocytic cells, granulocytes, and soluble macromolecules produced by the above cells or the liver (including antibodies, cytokines, and complement) that results in selective damage to, destruction of, or elimination from the human body of cancerous cells, metastatic tumor cells, malignant melanoma, invading pathogens, cells or tissues infected with pathogens, or, in cases of autoimmunity or pathological inflammation, normal human cells or tissues.

As used herein, the term “effective amount”, “therapeutically effective amount”, or the like of a composition, including modified interferon-α2 compounds or compositions of the present disclosure is a quantity sufficient to achieve a desired therapeutic and/or prophylactic effect, e.g., an amount that results in the prevention of, or a decrease in, the symptoms associated with a disease that is being treated. The amount of a compound or composition of the present disclosure administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compounds and compositions of the present disclosure can also be administered in combination with each other or with one or more additional therapeutic compounds.

As used herein, the term “T cell epitope” means an MHC ligand or protein determinant, 7 to 30 amino acids in length, and capable of specific binding to human leukocyte antigen (HLA) molecules and interacting with specific T cell receptors (TCRs). Generally, T cell epitopes are linear and do not express specific three-dimensional characteristics. T cell epitopes are not affected by the presence of denaturing solvents. The ability to interact with T cell epitopes can be predicted by in silico methods (De Groot A S et al., (1997), AIDS Res Hum Retroviruses, 13(7):539-41; Schafer J R et al., (1998), Vaccine, 16(19):1880-4; De Groot A S et al., (2001), Vaccine, 19(31):4385-95; De Groot A R et al., (2003), Vaccine, 21(27-30):4486-504, all of which are herein incorporated by reference in their entirety.

As used herein, the term “T-cell epitope cluster” refers to polypeptide that contains between about 4 to about 40 MHC binding motifs. In particular embodiments, the T-cell epitope cluster contains between about 5 to about 35 MHC binding motifs, between about 8 and about MHC binding motifs; and between about 10 and 20 MHC binding motifs.

As used herein, the term “immune stimulating T-cell epitope polypeptide” refers to a molecule capable of inducing an immune response, e.g., a humoral, T cell-based, or innate immune response.

As used herein, the term “B cell epitope” means a protein determinant capable of specific binding to an antibody. B cell epitopes usually consist of chemically active surface groupings of molecules such as amino acids or sugar side chains and usually have specific three-dimensional structural characteristics, as well as specific charge characteristics. Conformational and non-conformational epitopes are distinguished in that the binding to the former but not the latter is lost in the presence of denaturing solvents.

The term “subject” as used herein refers to any living organism in which an immune response is elicited. The term subject includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

As used herein, the term “MHC complex” refers to a protein complex capable of binding with a specific repertoire of polypeptides known as HLA ligands and transporting said ligands to the cell surface.

As used herein, the term “MHC Ligand” means a polypeptide capable of binding to one or more specific MHC alleles. The term “HLA ligand” is interchangeable with the term “MHC Ligand”.

Cells expressing MHC/Ligand complexes on their surface are referred to as “Antigen Presenting Cells” (APCs).

As used herein, the term “T Cell Receptor” or “TCR” refers to a protein complex expressed by T cells that is capable of engaging a specific repertoire of MHC/Ligand complexes as presented on the surface of APCs.

As used herein, the term “MHC Binding Motif” refers to a pattern of amino acids in a protein sequence that predicts binding to a particular MHC allele.

As used herein, the term “EpiBar™” refers to a 9-mer peptide that is predicted to be reactive to at least four different HLA alleles.

As used herein, the term “Immune Synapse” means the protein complex formed by the simultaneous engagement of a given T cell epitope to both a cell surface MHC complex and TCR.

The term “polypeptide” refers to a polymer of amino acids, and not to a specific length; thus, peptides, oligopeptides and proteins are included within the definition of a polypeptide. As used herein, a polypeptide is said to be “isolated” or “purified” when it is substantially free of cellular material when it is isolated from recombinant and non-recombinant cells, or free of chemical precursors or other chemicals when it is chemically synthesized. A polypeptide (e.g., a modified IFNα-2 polypeptide) of the present disclosure, however, can be joined to, linked to, or inserted into another polypeptide (e.g., a heterologous polypeptide) with which it is not normally associated in a cell and still be “isolated” or “purified.” When a polypeptide is recombinantly produced, it can also be substantially free of culture medium, for example, culture medium represents less than about 20%, less than about 10%, or less than about 5% of the volume of the polypeptide preparation.

The terms “polynucleotide” and “nucleic acid sequence” are used interchangeably to refer to a deoxyribonucleotide or ribonucleotide polymer in either single- or double-stranded form, and unless otherwise limited, encompasses known analogues (e.g., peptide nucleic acids) having the essential nature of natural nucleotides in that they hybridize to single-stranded nucleic acids in a manner similar to naturally occurring nucleotides. The term “polynucleotide” is not intended to limit the present invention to polynucleotides comprising DNA. Those of ordinary skill in the art will recognize that polynucleotides, can comprise ribonucleotides and combinations of ribonucleotides and deoxyribonucleotides. Such deoxyribonucleotides and ribonucleotides include both naturally occurring molecules and synthetic analogues. The polynucleotides of the invention also encompass all forms of sequences including, but not limited to, single-stranded forms, double-stranded forms, and the like. As used herein, the terms “encoding” or “encoded” when used in the context of a specified polynucleotide mean that the polynucleotide comprises the requisite information to direct translation of the polynucleotide sequence into a specified polypeptide. The information by which a polypeptide is encoded is specified by the use of codons.

A polynucleotide encoding a polypeptide may comprise non-translated sequences (e.g., introns) within translated regions of the nucleic acid or may lack such intervening non-translated sequences (e.g., as in cDNA).

As used herein, the term “natural interferon,” “natural human interferon-alpha 2b” (hIFN-α2b), “natural human interferon-alpha 2a” (hIFN-α2a), “natural human interferon-alpha 2c” (hIFN-α2c) “wild type interferon,” “native interferon,” or variants thereof refers to a cytokine (e.g., polypeptide, nucleic acid, etc.) as it is found in nature (i.e., wild type), without having been subjected to any kind of artificial modification or mutation.

As used herein, the term “amino acid substitution” refers to the change of one amino acid in the primary sequence of a natural (i.e., wild type) protein, such as hIFN-α2, for another amino acid.

As used herein, the term “modified interferon-α2,” “modified interferon-α2,” “glycosylated modified interferon-α2,” “human modified interferon-α2 with reduced immunogenicity,” “modified GMOP-interferon-α2,” “modified IFN-α2,” “modified GMOP-IFN-α2,” “modified interferon-alpha-2,” “modified GMOP-interferon-alpha-2,” “modified IFN-alpha-2,” “modified GMOP-IFN-alpha-2,” “modified interferon-2,” “modified GMOP-interferon-2,” “modified IFN-2,” “modified GMOP-IFN-2,” or variants thereof refers to molecules of a modified interferon alpha 2 molecule, containing changes to the amino acid or nucleic acid sequence as compared to the appropriate natural interferon, and in aspects includes at least one glycosylation site, with or without a GMOP amino acid sequence attached. In aspects, said molecules have reduced immunogenicity as compared to natural human interferon.

As used herein, the term “GMOP” refers to an amino acid sequence (SEQ. ID NO: 26) of a peptide derived from human granulocyte and macrophage-colony stimulating factor (GM-CSF) that contains four potential O-glycosylation sites, as well as a nucleic acid sequence (SEQ. ID NO: 25) that encodes for the GMOP peptide. “GMOP” may refer to a GMOP amino acid and/or nucleic acid sequence by itself, and/or as a component of larger amino acid and/or nucleic acid sequence.

As used herein, the term “hyperglycosylated” refers to a molecule comprising more than three additional glycosylations to those of native interferon-α2. Preferably, the glycosylated modified interferon-α2 of the present disclosure is hyperglycosylated, and comprises between 4 and 6 additional glycosylations than are present in the native interferon.

As used herein, the term “O-glycosylation site” refers to a serine of threonine residue within an amino acid sequence that is susceptible to O-glycosylation. The “position” of the “O-glycosylation site” is indicated by the position of a serine or threonine residue that is susceptible to O-glycosylation in the amino acid sequence. Said Ser or Thr residue, in said sequence, may be subjected to O-type enzymatic glycosylation, such as by O-glycosyltransferases. It is understood that there is a lack of known consensus recognition sequences for O-glycosyltransferases, although some O-glycosylation sites for specific proteins are known.

As used herein, the term “N-glycosylation site” refers to an Asn-Xaa-Ser/Thr tripeptide, where X may be any residue except a proline residue. The “position” of the “N-glycosylation site” is indicated by the position occupied by an amino acid residue in the amino acid sequence of a natural human interferon-alpha 2b that will be replaced by an Asn or it is the asparagine of said consensus sequence. Said Asn residue, in said consensus sequence, may be subjected to an N-type enzymatic glycosylation.

As used herein, the term “PEGylation” refers to the addition of one or more PEG (polyethylene glycol) polymer chains to a molecule (e.g., a polypeptide). PEGylation may be achieved by covalent and/or non-covalent attachment, and/or by covalent and/or non-covalent amalgamation of a PEG polymer chain to a molecule. “PEGylated” refers to molecules that have undergone PEGylation (i.e., one or more PEG polymer chains have been added to the molecule).

As used herein, the term “z-score” indicates how many standard deviations an element is from the mean. A z-score can be calculated from the following formula. z=(X−μ)/σ where z is the z-score, X is the value of the element, p is the population mean, and a is the standard deviation.

The following abbreviations and/or acronyms are used throughout this application:

ADA antibody developed against
APC antigen presenting cells
DMSO dimethyl sulfoxide
DR antibody antigen D related antibody
EDTA ethylenediaminetetraacetic acid
ELISA enzyme-linked immunosorbent assay
HLA human leukocyte antigen
IFN interferon
MHC major histocompatibility complex
PBMC peripheral blood mononuclear cell
RPMI Roswell Park Memorial Institute medium
TCR T-cell receptor
T_{eff} effector T cell
Tₕ helper T cell
T_{Reg} regulatory T cell

### Modified IFN-α2 Polypeptides and Nucleic Acids

In aspects, the present disclosure provides modified IFNα-2 polypeptides (including modified IFNα-2b polypeptides, modified IFNα-2a polypeptides, and IFNα-2c polypeptides) with proven antiviral biological activity, improved pharmacokinetic parameters with respect to wild-type and commercial IFNα-2 cytokines (e.g., INTRON-A, PEGINTRON, SYLATRON), and reduced immunogenicity, and thus have use in human therapy, including human antiviral therapy.

In aspects, the present disclosure provides a modified interferon-α2 polypeptide or nucleic acid having interferon-α2 activity (e.g., anti-viral activity) and reduced immunogenicity. In aspects, the modifications carried out in the natural amino acid sequence of human interferon-α2 for obtaining the modified interferon-α2 of the disclosure are a result of a modification of the amino acid encoding natural human interferon or a modification of a gene encoding natural human interferon, such as hIFN-alpha-2a, hIFN-alpha-2b, and hIFN-alpha-2c. In aspects, the modifications carried out in the natural amino acid sequence of human interferon-α2, optionally with the GMOP peptide sequence (or a fragment thereof) added on the N-terminus and/or C-terminus of the sequence of human interferon-α2, for obtaining the modified GMOP-interferon-α2 of the disclosure are a result of a modification of the amino acid encoding natural human interferon or a modification of a gene encoding such, such as wild type GMOP-IFN-alpha-2a, wild type GMOP-IFN-alpha-2b, and wild type GMOP-IFN-alpha-2c. Further, said modifications are introduced in such a way that they reduce the immunogenicity of the amino acid sequence as compared to natural human interferon, while maintaining its biological activity (such as its antiviral biological activity).

In aspects, the modified interferon-α2 polypeptides and related modified interferon-α2 compounds and compositions of the present disclosure have reduced immunogenicity as compared to natural interferon-α2. Mutations that reduce the immunogenicity of a modified interferon-α2 as compared to natural interferon-α2 were identified by EpiMatrix™ analysis. EpiMatrix™ is a proprietary computer algorithm developed by EpiVax (Providence, R.I.), which is used to screen protein sequences for the presence of putative T cell epitopes. Input sequences are parsed into overlapping 9-mer frames where each frame overlaps the last by 8 amino acids. Each of the resulting frames is then scored for predicted binding affinity with respect to a panel of eight common Class II HLA alleles (DRB1*0101, DRB1*0301, DRB1*0401, DRB1*0701, DRB1*0801, DRB1*1101, DRB1*1301, and DRB1*1501). Raw scores are normalized against the scores of a large sample of randomly generated peptides. The resulting “Z” score is reported. In aspects, any 9-mer peptide with an allele-specific EpiMatrix™ Z-score in excess of 1.64, theoretically the top 5% of any given sample, is considered a putative T cell epitope.

Peptides containing clusters of putative T cell epitopes are more likely to test positive in validating in vitro and in vivo assays. The results of the initial EpiMatrix™ analysis are further screened for the presence of putative T cell epitope “clusters” using a second proprietary algorithm known as Clustimer™ algorithm. The Clustimer™ algorithm identifies sub-regions contained within any given amino acid sequence that contains a statistically unusually high number of putative T cell epitopes. Typical T-cell epitope “clusters” range from about 9 to roughly amino acids in length and, considering their affinity to multiple alleles and across multiple 9-mer frames, can contain anywhere from about 4 to about 40 putative T cell epitopes. Each epitope cluster identified an aggregate EpiMatrix™ score is calculated by summing the scores of the putative T cell epitopes and subtracting a correcting factor based on the length of the candidate epitope cluster and the expected score of a randomly generated cluster of the same length. EpiMatrix™ cluster scores in excess of +10 are considered significant. In aspects, modified interferon-α2 molecules of the instant disclosure contain one or more modifications (e.g., changes, substitutions, or mutations) in the T cell epitope clusters to reduce their immunogenicity. For example, modified interferon-α2 mutations for the instantly-disclosed modified interferon α2 molecules are selected that not only reduce the immunogenicity of the molecule, but also do not significantly reduce its biological activity, such as its antiviral activity, and/or that do not affect its binding to receptors involved in the interferon's biological activity. In aspects, such modifications for modified interferon-α2 molecules of the present disclosure are selected that do not disrupt the structure or function of the natural interferon and include substitution of one or more amino acids occupying select positions in the natural human interferon-alpha-2 for alanine, threonine, or glycine.

Many of the most reactive T cell epitope clusters contain a feature referred to as an “EpiBar™”. As described previously, an EpiBar™ is a single 9-mer frame that is predicted to be reactive to at least four different HLA alleles. In aspects, the modified interferon-α2 molecules of the present disclosure can comprise one or more modifications (e.g., changes, substitutions, or mutations) within the EpiBars® of the natural interferon-α2. In aspects, said modifications of the modified interferon-α2 molecules reduce the immunogenicity of the modified interferon-α2 molecules as compared to the natural IFN-α2. In aspects, said modifications of the modified interferon-α2 molecules additionally do not disrupt the structure or function of the natural interferon-α2 activity. For example, modified interferon-α2 mutations are selected that do not significantly reduce its biological activity, such as its antiviral activity, and/or that do not affect its binding to receptors involved in the interferon's biological activity. In aspects, such modifications for modified interferon-α2 molecules of the present disclosure are selected that do not disrupt the structure or function of the natural interferon and include substitution of one or more amino acids occupying select positions in the natural human interferon-alpha-2 for alanine, threonine, or glycine.

In aspects, the contribution of each amino acid in these identified cluster regions to HLA binding was evaluated using OptiMatrix tool (part of the EpiVax ISPRI toolkit for deimmunization). OptiMatrix begins with looking at “critical” residues, which contribute most to MHC binding affinity across multiple 9-mer frames and multiple HLA alleles. The program then iteratively substitutes all 19 alternative amino acids in any given position of a protein sequence (with operator-defined input that may limit the list to naturally conserved variants) and then re-analyzes the predicted immunogenicity of the sequence following that change. To avoid a negative effect on protein structure and consequently in biological activity, a comprehensive search in literature for critical residues was also conducted, which identified amino acids that were not candidates for modification. In aspects, said modifications of the modified interferon-α2 molecules reduce the immunogenicity of the modified interferon-α2 molecules as compared to the natural IFN-α2. In aspects, said modifications of the modified interferon-α2 molecules additionally do not disrupt the structure or function of the natural interferon-α2 activity. For example, modified interferon-α2 mutations are selected that do not significantly reduce its biological activity, such as its antiviral activity, and/or that do not affect its binding to receptors involved in the interferon's biological activity. In aspects, such modifications for modified interferon-α2 molecules of the present disclosure are selected that do not disrupt the structure or function of the natural interferon and include substitution of one or more amino acids occupying select positions in the natural human interferon-alpha-2 for alanine, threonine, or glycine.

In aspects, a modified interferon-α2 polypeptide comprises the substitution of one or more amino acids occupying positions selected from the group consisting of the following positions in the natural human interferon-alpha-2 (including interferon-alpha-2b (SEQ ID NO: 12), interferon-alpha-2a (SEQ ID NO: 22), and interferon-alpha-2c (SEQ ID NO: 24): 9, 17, 47, 65, 66, 117, 123, 128, 147, and 157. In aspects, a modified interferon-α2 polypeptide comprises the substitution of one or more amino acids occupying positions selected from the group consisting of the following positions in the natural human interferon-alpha-2 (including interferon-alpha-2b (SEQ ID NO: 12), interferon-alpha-2a (SEQ ID NO: 22), and interferon-alpha-2c (SEQ ID NO: 24): 9, 17, 47, 65, 66, 117, 123, 128, 147, and 157, where such substitution includes the change of the amino acid from that position to an amino acid selected from the group consisting of: alanine, glycine, or threonine. In aspects, a modified interferon-α2 polypeptide comprises the substitution of one or more amino acids occupying positions selected from the group consisting of the following positions in the natural human interferon-alpha-2 (including interferon-alpha-2b (SEQ ID NO: 12), interferon-alpha-2a (SEQ ID NO: 22), and interferon-alpha-2c (SEQ ID NO: 24): 9, 17, 47, 65, 66, 117, 123, 128, 147, and 157, where such mutations reduce the immunogenicity of the modified interferon-α2 polypeptide as compared to the natural human interferon-alpha-2. In aspects, a modified interferon-α2 molecule is a modified interferon-alpha-2b polypeptide. In aspects, a modified interferon-α2 polypeptide is a modified IFN-α2a polypeptide. In aspects, a modified interferon-α2 polypeptide is a modified IFN-α2c polypeptide. In aspects, the modified interferon-α2 polypeptides as described herein are hyperglycosylated. In aspects of the above-described polypeptides, the modified interferon-α2 polypeptides may be isolated, synthetic, or recombinant.

In aspects, a modified interferon-α2 polypeptide as described herein is hyperglycosylated. Glycosylation of certain eukaryotic proteins takes place at certain positions of the polypeptide backbone, and commonly there are two types of glycosylation. O-type glycosylation involves binding of an oligosaccharide to an “—OH” (hydroxyl) group of a serine or threonine residue. N-type glycosylation involves binding of an oligosaccharide to an “—NH” group of an Asparagine residue. Particularly, N-glycosylation takes place in the consensus sequence, Asn-X-Ser/Thr, where X may be any amino acid different from Proline. All the oligosaccharides bound to a protein through an N-type binding have a pentasaccharide nucleus in common comprised by three mannose residues and two N-acetylglucosamine residues. Any sugars bound to this pentasaccharide nucleus may acquire a great variety of oligosaccharide patterns. The presence or absence of said oligosaccharides affects the physical properties of proteins and may be critical in their function, stability, secretion, and location in the cell. In aspects, a modified interferon-α2 polypeptide comprises the addition of amino acids containing one or more sites of N or O glycosylation.

In aspects, a modified interferon-α2 polypeptides as described herein comprise a peptide sequence called GMOP, sequence APARSPSPSTQPWE (SEQ ID NO: 26) or a fragment thereof, conjugated to a modified interferon-α2 sequence. In aspects, said fragment of APARSPSPSTQPWE is at least 7, at least 8, at least 9 and/or at least 10 amino acids in length. GMOP is a 14-amino acid-long peptide (SEQ. ID NO: 26) derived from the N-terminal region of the stimulating factor of granulocyte colonies and human macrophages (hGM-CSF), a stimulating growth factor of the proliferation and maturation of hematopoietic progenitors of various cell lineages, secreted by a wide variety of cells (endothelial cells, fibroblasts, macrophages, T cells, mast cells) in response to specific signals, which acts in a paracrine manner. hGM-CSF is a monomeric glycoprotein that, in its mature form, consists of 127 amino acids and exhibits a molecular mass between 14.5 and 32 kDa. This heterogeneity in its molecular mass is due to the two potential sites of N-glycosylation in residues N44 and N54 and 4 potential sites of O-glycosylation in the N-Terminal region: residues S22, S24, S26 and T27 (which correlate to residues S5, S7, S9, and T10 in the mature form of hGM-CSF, respectively). The first 7 amino acids (APARSPS) of mature hGM-CSF are a linear epitope, capable of being recognized by an anti-hGM-CSF monoclonal antibody (called, mAb CC1H7). The interaction of this epitope with its corresponding paratope has the characteristic of modifying its affinity with variations of ion strength, representing the latter an operational advantage for the development of immunochemical techniques, such as enzyme linked immunosorbent assay (ELISA), immunoaffinity chromatography, and western blot, among others (Perotti, Oggero, Etcheverrigaray, and Kratje, AR057215A1).

The addition of said GMOP sequence gives between 4 and 6 additional O-glycosylation sites to the molecule of the present invention. In this way, a modified interferon-α2, to which one or more GMOP peptide sequences (APARSPSPSTQPWE) or fragment thereof have been added, is referred to as modified GMOP-interferon-α2 (it may also be referred to as, for example, GMOP-IFN-α2, etc.). The addition of this peptide sequence is done using any of the techniques known in the state of the art. In embodiments, said GMOP peptide sequence or label (APARSPSPSTQPWE) or a fragment thereof can be placed at the terminal amino end of a modified interferon-α2 polypeptide sequence and/or at the terminal carboxyl end of a modified interferon-α2 sequence. In aspects, said fragment of APARSPSPSTQPWE is at least 7, at least 8, at least 9 and/or at least 10 amino acids in length. In preferred embodiments, the GMOP peptide sequence (SEQ ID NO: 26) is added onto the N-terminal end of a modified interferon-α2 sequence.

In aspects, a modified interferon-α2 also comprises the addition of amino acids containing one or more sites of N or O glycosylation, wherein these added amino acids comprise one or more sequences with at least 60%, 70%, 80%, 90%, or 95% homology to APARSPSPSTQPWE or a fragment thereof. In aspects, a modified interferon-α2 as disclosed herein include the addition of one or more of the amino acid sequence APARSPSPSTQPWE (SEQ ID NO: 26) or a fragment thereof. In aspects, said modified interferon-α2 comprises the addition of amino acids containing one or more sites of N or O glycosylation, wherein these added amino acids comprise one or more sequences with at least 70%, 80%, or 90% homology to APARSPSPSTQPWE (SEQ ID NO: 26) or a fragment thereof, and wherein the amino acids at positions 5, 7, 9, and 10 of SEQ ID NO: 26 are not substituted. In aspects, said above described amino acids containing one or more sites of N or O glycosylation (for example, said one or more sequences with at least 60%, 70%, 80%, 90%, or 95% homology to APARSPSPSTQPWE) or a fragment thereof may be added to the N and/or C-terminus of the instantly-disclosed modified interferon-α2 polypeptides. In aspects, said fragment of APARSPSPSTQPWE is at least 5, at least 6, at least 7, at least 8, at least 9 and/or at least 10 amino acids in length. In aspects of the above-described polypeptides, the modified interferon-α2 polypeptides may be isolated, synthetic, or recombinant.

### Modified Interferon-α2b Polypeptides and Nucleic Acids

In aspects, a modified interferon-α2 polypeptide of the present disclosure is a modified interferon-α2b polypeptide having interferon-α2b activity and a reduced immunogenicity or a reduced propensity to elicit an immune response as compared to a wild type interferon-α2b polypeptide of SEQ ID NO: 12. In aspects, a modified interferon-α2b polypeptide comprises an amino acid sequence with at least 60%, 70%, 80%, 90%, or 95% homology to wild type interferon-α2b (SEQ ID NO: 12) and further comprises one or more amino acid substitutions in any of the positions selected from the set comprised of: 9, 17, 47, 65, 66, 117, 123, 128, 147, and 157. In aspects, a modified interferon-α2b polypeptide comprises an amino acid sequence with at least 60%, 70%, 80%, 90%, or 95% homology to wild type interferon-α2b (SEQ ID NO: 12) and further comprises one or more amino acid substitutions in any of the positions selected from the set comprised of: 9, 17, 47, 65, 66, 117, 123, 128, 147, and 157, wherein said substitution comprises the change of the amino acid of said position to alanine, glycine, or threonine. In aspects, a modified interferon-α2b polypeptide comprises an amino acid sequence with at least 60%, 70%, 80%, 90%, or 95% homology to wild type interferon-α2b (SEQ ID NO: 12) and further comprises at least five amino acid substitutions in any of the positions selected from the set comprised of: 9, 17, 47, 65, 66, 117, 123, 128, 147, and 157. In aspects, a modified interferon-α2b polypeptide comprises an amino acid sequence with at least 60%, 70%, 80%, 90%, or 95% homology to wild type interferon-α2b (SEQ ID NO: 12) and further comprises at least five amino acid substitutions in any of the positions selected from the set comprised of: 9, 17, 47, 65, 66, 117, 123, 128, 147, and 157, wherein said substitution comprises the change of the amino acid of said position to alanine, glycine, or threonine. In aspects of the above-described polypeptide, the modified interferon-α2b polypeptide may be isolated, synthetic, or recombinant.

In aspects, a modified interferon-α2b having interferon-α2b activity polypeptide having interferon-α2b activity comprises an amino acid sequence with at least 60%, 70%, 80%, 90%, or 95% homology to wild type interferon-α2b (SEQ ID NO: 12) and further comprises amino acid substitutions at positions 9, 47, 117, 123, and 128, wherein said substitutions comprise the change of the amino acid of said position to alanine, glycine, or threonine. In aspects, a modified interferon-α2b polypeptide having interferon-α2b activity comprises an amino acid sequence with at least 60%, 70%, 80%, 90%, or 95% homology to wild type interferon-α2b (SEQ ID NO: 12) and further comprises the mutations L9A, F47A, L117A, F123A, and L128A. In aspects of the above-described polypeptides, the modified interferon-α2b polypeptide have reduced immunogenicity or a reduced propensity to elicit an immune response as compared to a wild type interferon-α2b polypeptide of SEQ ID NO: 12. In aspects of the above-described polypeptides, the modified interferon-α2b polypeptide may be isolated, synthetic, or recombinant.

In aspects, a modified interferon-α2b polypeptide having interferon-α2b activity comprises an amino acid sequence with at least 60%, 70%, 80%, 90%, or 95% homology to wild type interferon-α2b (SEQ ID NO: 12) and further comprises amino acid substitutions at positions 9, 47, 117, 123, 128, 147, and 157, wherein said substitutions comprise the change of the amino acid of said position to alanine, glycine, or threonine. In aspects, a modified interferon-α2b polypeptide having interferon-α2b activity comprises an amino acid sequence with at least 60%, 70%, 80%, 90%, or 95% homology to wild type interferon-α2b (SEQ ID NO: 12) and further comprises the mutations L9A, F47A, L117A, F123A, L128A, I147T, and L157A. In aspects of the above-described polypeptides, the modified interferon-α2b polypeptide have reduced immunogenicity or a reduced propensity to elicit an immune response as compared to a wild type interferon-α2b polypeptide of SEQ ID NO: 12. In aspects of the above-described polypeptides, the modified interferon-α2b polypeptide may be isolated, synthetic, or recombinant.

In aspects, a modified interferon-α2b polypeptide having interferon-α2b activity comprises an amino acid sequence with at least 60%, 70%, 80%, 90%, or 95% homology to wild type interferon-α2b (SEQ ID NO: 12) and further comprises amino acid substitutions at positions 9, 47, 65, 66, 117, 123, and 128, wherein said substitutions comprise the change of the amino acid of said position to alanine, glycine, or threonine. In aspects, a modified interferon-α2b polypeptide having interferon-α2b activity comprises an amino acid sequence with at least 60%, 70%, 80%, 90%, or 95% homology to wild type interferon-α2b (SEQ ID NO: 12) and further comprises the mutations L9A, F47A, N65A, L66A, L117A, F123A, and L128A. In aspects of the above-described polypeptides, the modified interferon-α2b polypeptide have reduced immunogenicity or a reduced propensity to elicit an immune response as compared to a wild type interferon-α2b polypeptide of SEQ ID NO: 12. In aspects of the above-described polypeptides, the modified interferon-α2b polypeptide may be isolated, synthetic, or recombinant.

In aspects, a modified interferon-α2b polypeptide having interferon-α2b activity comprises an amino acid sequence with at least 60%, 70%, 80%, 90%, or 95% homology to wild type interferon-α2b (SEQ ID NO: 12) and further comprises amino acid substitutions at positions 9, 17, 47, 65, 66, 117, 123, 128, 147, and 157, wherein said substitutions comprise the change of the amino acid of said position to alanine, glycine, or threonine. In aspects, a modified interferon-α2b polypeptide having interferon-α2b activity comprises an amino acid sequence with at least 60%, 70%, 80%, 90%, or 95% homology to wild type interferon-α2b (SEQ ID NO: 12) and further comprises the mutations L9A, L17A, F47A, N65A, L66A, L117A, F123A, L128A, I147T, and L157A. In aspects of the above-described polypeptides, the modified interferon-α2b polypeptide have reduced immunogenicity or a reduced propensity to elicit an immune response as compared to a wild type interferon-α2b polypeptide of SEQ ID NO: 12. In aspects of the above-described polypeptides, the modified interferon-α2b polypeptide may be isolated, synthetic, or recombinant.

In aspects, a modified interferon-α2b polypeptide having interferon-α2b activity is selected from the group consisting of SEQ ID NO: 14, SEQ ID NO: 16, SEQ ID NO: 18, and SEQ ID NO: 20. In aspect, a modified interferon-α2b polypeptide having interferon-α2b activity is selected from the group consisting of: SEQ ID NO: 16 and SEQ ID NO: 18. In aspects, a modified interferon-α2b polypeptide comprises an amino acid sequence of SEQ ID NO: 14. In aspects, a modified interferon-α2b polypeptide comprises an amino acid sequence of SEQ ID NO: 20. In a preferred embodiment, a modified interferon-α2b polypeptide comprises an amino acid sequence of SEQ ID NO: 16. In aspects, a modified interferon-α2b polypeptide comprises an amino acid sequence of SEQ ID NO: 18. In aspects of the above-described polypeptides, the modified interferon-α2b polypeptides have reduced immunogenicity or a reduced propensity to elicit an immune response as compared to a wild type interferon-α2b polypeptide of SEQ ID NO: 12. In aspects of the above-described polypeptides, the modified interferon-α2b polypeptide may be isolated, synthetic, or recombinant.

In aspects, the instantly-disclosed modified interferon-α2b polypeptides having interferon-α2b activity, such as the above-described modified interferon-α2b polypeptides, have a relative antiviral activity of between 5% and 95% as compared to a wild type interferon-α2b polypeptide of SEQ ID NO: 12. In aspects, the instantly-disclosed modified interferon-α2b polypeptides having interferon-α2b activity, such as the above-described modified interferon-α2b polypeptides, have a relative antiviral activity of between 10% and 90% as compared to a wild type interferon-α2b polypeptide of SEQ ID NO: 12. In aspects, the instantly-disclosed modified interferon-α2b polypeptides having interferon-α2b activity, such as the above-described modified interferon-α2b polypeptides, have a relative antiviral activity of between 20% and 80% as compared to a wild type interferon-α2b polypeptide of SEQ ID NO: 12.

In aspects, the instantly-disclosed modified interferon-α2b polypeptides having interferon-α2b activity, such as the above-described modified interferon-α2b polypeptides, have a percentage antiproliferative biological activity of between 0% and 50%. In aspects, a modified interferon-α2b polypeptide having interferon-α2b activity, such as the above-described modified interferon-α2b polypeptides, has a percentage antiproliferative biological activity of less than 10%. In aspects, a modified interferon-α2b polypeptide having interferon-α2b activity, such as the above-described modified interferon-α2b polypeptides, has a percentage antiproliferative biological activity of less than 5%.

In aspects, the instantly-disclosed modified interferon-α2b polypeptides having interferon-α2b activity, such as the above-described modified interferon-α2b polypeptides, have an apparent plasma clearance rate (Clₐₚₚ) of between 5 mL/h-200 mL/h. In aspects, a modified interferon-α2b polypeptide having interferon-α2b activity, such as the above-described modified interferon-α2b polypeptides, has an apparent plasma clearance rate (Clₐₚₚ) of less than 115 mL/h. In aspects, a modified interferon-α2b polypeptide having interferon-α2b activity, such as the above-described modified interferon-α2b polypeptides, has an apparent plasma clearance rate (Clₐₚₚ) of less than 50 mL/h.

In aspects, the present disclosure provides a polynucleotide or nucleic acid (e.g., DNA, including cDNA or RNA, including mRNA) encoding a modified interferon-α2b polypeptide having interferon-α2b activity, such as the above-described modified interferon-α2b polypeptides. For example, in aspects, the present disclosure provides a nucleic acid encoding for one or more modified interferon-α2b polypeptides selected from the group consisting of: SEQ ID NO: 14, SEQ ID NO: 16, SEQ ID NO: 18, and SEQ ID NO: 20. In aspects, the present disclosure provides a nucleic acid encoding for one or more modified interferon-α2b polypeptides selected from the group consisting of: SEQ ID NO: 16 and SEQ ID NO: 18. In aspects, a nucleic acid encoding for one or more one or more modified interferon-α2b polypeptides comprises one or more nucleic acid sequences selected from the group consisting of: SEQ ID NO: 13, SEQ ID NO: 15, SEQ ID NO: 17, and SEQ ID NO: 19. In aspects, a nucleic acid encoding for one or more one or more modified interferon-α2b polypeptides comprises one or more nucleic acid sequences selected from the group consisting of: SEQ ID NO: 15 and SEQ ID NO: 17. In aspects, a nucleic acid encoding a modified interferon-α2b polypeptide comprises a nucleic acid sequence of SEQ ID NO: 13. In aspects, a nucleic acid encoding a modified interferon-α2b polypeptide comprises a nucleic acid sequence of SEQ ID NO: 19. In a preferred embodiment, a nucleic acid encoding a modified interferon-α2b polypeptide comprises a nucleic acid sequence of SEQ ID NO: 15. In a preferred embodiment, a nucleic acid encoding a modified interferon-α2b polypeptide comprises a nucleic acid sequence of SEQ ID NO: 17.

In aspects, a modified interferon-α2b also comprises the addition of amino acids containing one or more sites of N or O glycosylation, wherein these added amino acids comprise one or more sequences with at least 60%, 70%, 80%, 90%, or 95% homology to APARSPSPSTQPWE or a fragment thereof. In aspects, a modified interferon-α2b as disclosed herein include the addition of one or more of the amino acid sequence APARSPSPSTQPWE (SEQ ID NO: 26) or a fragment thereof. In aspects, said modified interferon-α2b comprises the addition of amino acids containing one or more sites of N or O glycosylation, wherein these added amino acids comprise one or more sequences with at least 70%, 80%, or 90% homology to APARSPSPSTQPWE (SEQ ID NO: 26) or a fragment thereof, and wherein the amino acids at positions 5, 7, 9, and 10 of SEQ ID NO: 26 are not substituted. In aspects, said above described amino acids containing one or more sites of N or O glycosylation (for example, said one or more sequences with at least 60%, 70%, 80%, 90%, or 95% homology to APARSPSPSTQPWE) or a fragment thereof may be added to the N and/or C-terminus of the instantly-disclosed modified interferon-α2b polypeptides. In aspects, said fragment of APARSPSPSTQPWE is at least 5, at least 6, at least 7, at least 8, at least 9 and/or at least 10 amino acids in length. In aspects of the above-described polypeptides, the modified interferon-α2b polypeptides may be isolated, synthetic, or recombinant.

In aspects, a vector or plasmid comprising a nucleic acid of the present disclosure encoding one or more modified interferon-α2b polypeptides of the present disclosure, e.g., but not limited to, a nucleic acid (e.g., DNA or RNA) encoding at least modified interferon-α2b polypeptide having a sequence comprising, consisting of, or consisting essentially of one or more of SEQ. ID NO: 13, SEQ. ID NO: 15, SEQ. ID NO: 17, and SEQ. ID NO: 19, is provided. In aspects, the present disclosure is directed to a cell comprising a vector or plasmid of the present disclosure.

### Modified GMOP-Interferon-α2b Polypeptides and Nucleic Acids

In aspects, a modified interferon-α2 polypeptide of the present disclosure, including the modified IFNα-2b polypeptides described above, is a modified GMOP-interferon-α2b polypeptide having interferon-α2b activity and a reduced immunogenicity or a reduced propensity to elicit an immune response as compared to a wild type interferon-α2b polypeptide (SEQ ID NO: 12) and/or wild type GMOP-interferon-α2b (SEQ ID NO: 10). In aspects, a modified IFNα-2b polypeptide comprises the addition of amino acids containing one or more sites of N or O glycosylation, wherein these added amino acids comprise one or more sequences with at least 60%, 70%, 80%, 90%, or 95% homology to APARSPSPSTQPWE or a fragment thereof. In aspects, a modified IFNα-2b as disclosed herein includes the addition of one or more of the amino acid sequence APARSPSPSTQPWE (SEQ ID NO: 26) or a fragment thereof. In aspects, a modified IFNα-2b comprises the addition of amino acids containing one or more sites of N or O glycosylation, wherein these added amino acids comprise one or more sequences with at least 70%, 80%, or 90% homology to APARSPSPSTQPWE (SEQ ID NO: 26) or a fragment thereof, and wherein the amino acids at positions 5, 7, 9, and 10 of SEQ ID NO: 26 are not substituted. In aspects, said above described amino acids containing one or more sites of N or O glycosylation (for example, said one or more sequences with at least 60%, 70%, 80%, 90%, or 95% homology to APARSPSPSTQPWE) or a fragment thereof may be added to the N and/or C-terminus of the instantly-disclosed modified IFNα-2b polypeptides. In aspects, said fragment of APARSPSPSTQPWE is at least 5, at least 6, at least 7, at least 8, at least 9 and/or at least 10 amino acids in length.

In aspects, a modified GMOP-interferon-α2b polypeptide having interferon-α2b activity comprises an amino acid sequence with at least 60%, 70%, 80%, 90%, or 95% homology to wild type GMOP-interferon-α2b (SEQ ID NO: 10) and further comprises one or more amino acid substitutions in any of the positions selected from the set comprised of: 23, 31, 61, 79, 80, 131, 137, 142, 161, and 171. In aspects, a modified GMOP-interferon-α2b polypeptide having interferon-α2b activity comprises an amino acid sequence with at least 60%, 70%, 80%, 90%, or 95% homology to wild type interferon-α2b (SEQ ID NO: 10) and further comprises one or more amino acid substitutions in any of the positions selected from the set comprised of: 23, 31, 61, 79, 80, 131, 137, 142, 161, and 171, wherein said substitution comprises the change of the amino acid of said position to alanine, glycine, or threonine. In aspects, a modified GMOP-interferon-α2b polypeptide having interferon-α2b activity comprises an amino acid sequence with at least 60%, 70%, 80%, 90%, or 95% homology to wild type GMOP-interferon-α2b (SEQ ID NO: 10) and further comprises at least five amino acid substitutions in any of the positions selected from the set comprised of 23, 31, 61, 79, 80, 131, 137, 142, 161, and 171. In aspects, a modified GMOP-interferon-α2b polypeptide having interferon-α2b activity comprises an amino acid sequence with at least 60%, 70%, 80%, 90%, or 95% homology to wild type GMOP-interferon-α2b (SEQ ID NO: 10) and further comprises at least five amino acid substitutions in any of the positions selected from the set comprised of: 23, 31, 61, 79, 80, 131, 137, 142, 161, and 171, wherein said substitution comprises the change of the amino acid of said position to alanine, glycine, or threonine. In aspects of the above-described polypeptide, the modified GMOP-interferon-α2b polypeptide may be isolated, synthetic, or recombinant.

In aspects, a modified GMOP-interferon-α2b polypeptide having interferon-α2b activity comprises an amino acid sequence with at least 60%, 70%, 80%, 90%, or 95% homology to wild type GMOP-interferon-α2b (SEQ ID NO: 10) and further comprises one or more amino acid substitutions in any of the positions selected from the set comprised of: 23, 61, 131, 137, and 142, wherein said substitutions comprise the change of the amino acid of said position to alanine, glycine, or threonine. In aspects, a modified GMOP-interferon-α2b polypeptide having interferon-α2b activity comprises an amino acid sequence with at least 60%, 70%, 80%, 90%, or 95% homology to wild type GMOP-interferon-α2b (SEQ ID NO: 10) and further comprises the mutations L.23A, F61A, L131A, F137A, and L142A. In aspects of the above-described polypeptides, the modified GMOP-interferon-α2b polypeptide have reduced immunogenicity or a reduced propensity to elicit an immune response as compared to a wild type interferon-α2b polypeptide (SEQ ID NO: 12) and/or wild type GMOP-interferon-α2b (SEQ ID NO: 10). In aspects of the above-described polypeptides, the modified interferon-α2b polypeptide may be isolated, synthetic, or recombinant.

In aspects, a modified GMOP-interferon-α2b polypeptide having interferon-α2b activity comprises an amino acid sequence with at least 60%, 70%, 80%, 90%, or 95% homology to wild type GMOP-interferon-α2b (SEQ ID NO: 10) and further comprises one or more amino acid substitutions in any of the positions selected from the set comprised of 23, 61, 131, 137, 142, 161, and 171, wherein said substitutions comprise the change of the amino acid of said position to alanine, glycine, or threonine. In aspects, a modified GMOP-interferon-α2b polypeptide having interferon-α2b activity comprises an amino acid sequence with at least 60%, 70%, 80%, 90%, or 95% homology to wild type GMOP-interferon-α2b (SEQ ID NO: 10) and further comprises the mutations L23A, F61A, L131A, F137A, L142A, I161T, and L171A. In aspects of the above-described polypeptides, the modified GMOP-interferon-α2b polypeptide have reduced immunogenicity or a reduced propensity to elicit an immune response as compared to a wild type interferon-α2b polypeptide (SEQ ID NO: 12) and/or wild type GMOP-interferon-α2b (SEQ ID NO: 10). In aspects of the above-described polypeptides, the modified interferon-α2b polypeptide may be isolated, synthetic, or recombinant.

In aspects, a modified GMOP-interferon-α2b polypeptide having interferon-α2b activity comprises an amino acid sequence with at least 60%, 70%, 80%, 90%, or 95% homology to wild type GMOP-interferon-α2b (SEQ ID NO: 10) and further comprises one or more amino acid substitutions in any of the positions selected from the set comprised of: 23, 61, 79, 80, 131, 137, and 142, wherein said substitutions comprise the change of the amino acid of said position to alanine, glycine, or threonine. In aspects, a modified GMOP-interferon-α2b polypeptide having interferon-α2b activity comprises an amino acid sequence with at least 60%, 70%, 80%, 90%, or 95% homology to wild type GMOP-interferon-α2b (SEQ ID NO: 10) and further comprises the mutations L23A, F61A, N79A, L80A, L131A, F137A, and L142A. In aspects of the above-described polypeptides, the modified GMOP-interferon-α2b polypeptide have reduced immunogenicity or a reduced propensity to elicit an immune response as compared to a wild type interferon-α2b polypeptide (SEQ ID NO: 12) and/or wild type GMOP-interferon-α2b (SEQ ID NO: 10). In aspects of the above-described polypeptides, the modified interferon-α2b polypeptide may be isolated, synthetic, or recombinant.

In aspects, a modified GMOP-interferon-α2b polypeptide having interferon-α2b activity comprises an amino acid sequence with at least 60%, 70%, 80%, 90%, or 95% homology to wild type GMOP-interferon-α2b (SEQ ID NO: 10) and further comprises one or more amino acid substitutions in any of the positions selected from the set comprised of: 23, 31, 61, 79, 80, 131, 137, 142, 161, and 171, wherein said substitutions comprise the change of the amino acid of said position to alanine, glycine, or threonine. In aspects, a modified GMOP-interferon-α2b polypeptide having interferon-α2b activity comprises an amino acid sequence with at least 60%, 70%, 80%, 90%, or 95% homology to wild type GMOP-interferon-α2b (SEQ ID NO: 10) and further comprises the mutations L23A, L31A, F61A, N79A, L80A, I131A, F137A, L142A, I161T, and L171A. In aspects of the above-described polypeptides, the modified GMOP-interferon-α2b polypeptide have reduced immunogenicity or a reduced propensity to elicit an immune response as compared to a wild type interferon-α2b polypeptide (SEQ ID NO: 12) and/or wild type GMOP-interferon-α2b (SEQ ID NO: 10). In aspects of the above-described polypeptides, the modified interferon-α2b polypeptide may be isolated, synthetic, or recombinant.

In aspects, a modified GMOP-interferon-α2b polypeptide having interferon-α2b activity is selected from the group consisting of SEQ ID NO: 2, SEQ ID NO: 4, SEQ ID NO: 6, and SEQ ID NO: 8. In aspects, a modified GMOP-interferon-α2b polypeptide having interferon-α2b activity is selected from the group consisting of: SEQ ID NO: 4 and SEQ ID NO: 6. In aspects, a modified GMOP-interferon-α2b polypeptide comprises an amino acid sequence of SEQ ID NO: 2. In aspects, a modified GMOP-interferon-α2b polypeptide comprises an amino acid sequence of SEQ ID NO: 8. In aspects, a modified GMOP-interferon-α2b polypeptide comprises an amino acid sequence of SEQ ID NO: 4. In aspects, a modified GMOP-interferon-α2b polypeptide comprises an amino acid sequence of SEQ ID NO: 6. In aspects of the above-described polypeptides, the modified interferon-α2b polypeptides have reduced immunogenicity or a reduced propensity to elicit an immune response as compared to a wild type interferon-α2b polypeptide (SEQ ID NO: 12) and/or wild type GMOP-interferon-α2b (SEQ ID NO: 10). In aspects of the above-described polypeptides, the modified interferon-α2b polypeptide may be isolated, synthetic, or recombinant.

In aspects, a modified GMOP-interferon-α2b polypeptide having interferon-α2b activity, such as the above-described modified GMOP-interferon-α2b polypeptides, has a relative antiviral activity of between 5% and 95% as compared to a wild type interferon-α2b polypeptide (SEQ ID NO: 12) and/or wild type GMOP-interferon-α2b (SEQ ID NO: 10). In aspects, a modified GMOP-interferon-α2b polypeptide having interferon-α2b activity, such as the above-described modified GMOP-interferon-α2b polypeptides, has a relative antiviral activity of between 10% and 90% as compared to a wild type interferon-α2b polypeptide (SEQ ID NO: 12) and/or wild type GMOP-interferon-α2b (SEQ ID NO: 10). In aspects, a modified GMOP-interferon-α2b polypeptide having interferon-α2b activity, such as the above-described modified GMOP-interferon-α2b polypeptides, has a relative antiviral activity of between 20% and 80% as compared to a wild type interferon-α2b polypeptide (SEQ ID NO: 12) and/or wild type GMOP-interferon-α2b (SEQ ID NO: 10).

In aspects, the instantly-disclosed modified GMOP-interferon-α2b polypeptides having interferon-α2b activity, such as the above-described modified GMOP-interferon-α2b polypeptides, have a percentage antiproliferative biological activity of between 0% and 50%. In aspects, a modified GMOP-interferon-α2b polypeptide having interferon-α2b activity, such as the above-described modified GMOP-interferon-α2b polypeptides, has a percentage antiproliferative biological activity of less than 10%. In aspects, a modified GMOP-interferon-α2b polypeptide having interferon-α2b activity, such as the above-described modified GMOP-interferon-α2b polypeptides, has a percentage antiproliferative biological activity of less than 5%.

In aspects, the instantly-disclosed modified GMOP-interferon-α2b polypeptides having interferon-α2b activity, such as the above-described modified GMOP-interferon-α2b polypeptides, have an apparent plasma clearance rate (Clₐₚₚ) of between 5 mL/h-200 mL/h. In aspects, a modified GMOP-interferon-α2b polypeptide having interferon-α2b activity, such as the above-described modified GMOP-interferon-α2b polypeptides, has an apparent plasma clearance rate (Clₐₚₚ) of less than 115 mL/h. In aspects, a modified GMOP-interferon-α2b polypeptide having interferon-α2b activity, such as the above-described modified GMOP-interferon-α2b polypeptides, has an apparent plasma clearance rate (Clₐₚₚ) of less than 50 mL/h.

In aspects, the present disclosure provides a polynucleotide or nucleic acid (e.g., DNA, including cDNA or RNA, including mRNA) encoding a modified GMOP-interferon-α2b polypeptide having interferon-α2b activity, such as the above-described modified GMOP-interferon-α2b polypeptides. For example, in aspects, the present disclosure provides a nucleic acid encoding for one or more modified GMOP-interferon-α2b polypeptides selected from the group consisting of: SEQ ID NO: 2, SEQ ID NO: 4, SEQ ID NO: 6, and SEQ ID NO: 8. In aspects, the present disclosure provides a nucleic acid encoding for one or more modified GMOP-interferon-α2b polypeptides selected from the group consisting of: SEQ ID NO: 4 and SEQ ID NO: 6. In aspects, a nucleic acid encoding for one or more one or more modified GMOP-interferon-α2b polypeptides comprises one or more nucleic acid sequences selected from the group consisting of: SEQ ID NO: 1, SEQ ID NO: 3, SEQ ID NO: 5, and SEQ ID NO: 7. In a, a nucleic acid encoding for one or more one or more modified GMOP-interferon-α2b polypeptides comprises one or more nucleic acid sequences selected from the group consisting of: SEQ ID NO: 3 and SEQ ID NO: 5. In aspects, a nucleic acid encoding a modified GMOP-interferon-α2b polypeptide comprises a nucleic acid sequence of SEQ ID NO: 1. In aspects, a nucleic acid encoding a modified GMOP-interferon-α2b polypeptide comprises a nucleic acid sequence of SEQ ID NO: 7. In a preferred embodiment, a nucleic acid encoding a modified GMOP-interferon-α2b polypeptide comprises a nucleic acid sequence of SEQ ID NO: 3. In a preferred embodiment, a nucleic acid encoding a modified GMOP-interferon-α2b polypeptide comprises a nucleic acid sequence of SEQ ID NO: 5.

In aspects, a vector or plasmid comprising a nucleic acid of the invention encoding one or more modified GMOP-interferon-α2b polypeptides of the present disclosure, e.g., but not limited to, a nucleic acid (e.g., DNA or RNA) encoding at least one modified GMOP-interferon-α2b polypeptide having a sequence comprising, consisting of, or consisting essentially of one or more of: SEQ. ID NO: 1, SEQ. ID NO: 3, SEQ. ID NO: 5, and SEQ. ID NO: 7, is provided. In aspects, the present disclosure is directed to a cell comprising a vector or plasmid of the present disclosure.

### Modified Interferon-α2a Polypeptides and Nucleic Acids

In aspects, a modified interferon-α2 polypeptide of the present disclosure is a modified interferon-α2a polypeptide having interferon-α2a activity and a reduced immunogenicity or a reduced propensity to elicit an immune response as compared to a wild type interferon-α2a polypeptide of SEQ ID NO: 22. In aspects, a modified interferon-α2a polypeptide comprises an amino acid sequence with at least 60%, 70%, 80%, 90%, or 95% homology to wild type interferon-α2a (SEQ ID NO: 22) and further comprises one or more amino acid substitutions in any of the positions selected from the set comprised of: 9, 17, 47, 65, 66, 117, 123, 128, 147, and 157. In aspects, a modified interferon-α2a polypeptide comprises an amino acid sequence with at least 60%, 70%, 80%, 90%, or 95% homology to wild type interferon-α2a (SEQ ID NO: 22) and further comprises one or more amino acid substitutions in any of the positions selected from the set comprised of 9, 17, 47, 65, 66, 117, 123, 128, 147, and 157, wherein said substitution comprises the change of the amino acid of said position to alanine, glycine, or threonine. In aspects, a modified interferon-α2a polypeptide comprises an amino acid sequence with at least 60%, 70%, 80%, 90%, or 95% homology to wild type interferon-α2a (SEQ ID NO: 22) and further comprises at least five amino acid substitutions in any of the positions selected from the set comprised of: 9, 17, 47, 65, 66, 117, 123, 128, 147, and 157. In aspects, a modified interferon-α2a polypeptide comprises an amino acid sequence with at least 60%, 70%, 80%, 90%, or 95% homology to wild type interferon-α2a (SEQ ID NO: 22) and further comprises at least five amino acid substitutions in any of the positions selected from the set comprised of: 9, 17, 47, 65, 66, 117, 123, 128, 147, and 157, wherein said substitution comprises the change of the amino acid of said position to alanine, glycine, or threonine. In aspects of the above-described polypeptide, the modified interferon-α2a polypeptide may be isolated, synthetic, or recombinant.

In aspects, a modified interferon-α2a polypeptide having interferon-α2a activity comprises an amino acid sequence with at least 60%, 70%, 80%, 90%, or 95% homology to wild type interferon-α2a (SEQ ID NO: 22) and further comprises amino acid substitutions at positions 9, 47, 117, 123, and 128, wherein said substitutions comprise the change of the amino acid of said position to alanine, glycine, or threonine. In aspects, a modified interferon-α2a polypeptide having interferon-α2a activity comprises an amino acid sequence with at least 60%, 70%, 80%, 90%, or 95% homology to wild type interferon-α2a (SEQ ID NO: 22) and further comprises the mutations L9A, F47A, L117A, F123A, and L128A. In aspects of the above-described polypeptides, the modified interferon-α2a polypeptide has reduced immunogenicity or a reduced propensity to elicit an immune response as compared to a wild type interferon-α2a polypeptide of SEQ ID NO: 22. In aspects of the above-described polypeptides, the modified interferon-α2a polypeptide may be isolated, synthetic, or recombinant.

In aspects, a modified interferon-α2a polypeptide having interferon-α2a activity comprises an amino acid sequence with at least 60%, 70%, 80%, 90%, or 95% homology to wild type interferon-α2a (SEQ ID NO: 22) and further comprises amino acid substitutions at positions 9, 47, 117, 123, 128, 147, and 157, wherein said substitutions comprise the change of the amino acid of said position to alanine, glycine, or threonine. In aspects, a modified interferon-α2a polypeptide having interferon-α2a activity comprises an amino acid sequence with at least 60%, 70%, 80%, 90%, or 95% homology to wild type interferon-α2a (SEQ ID NO: 22) and further comprises the mutations L9A, F47A, L117A, F123A, L128A, I147T, and L157A. In aspects of the above-described polypeptides, the modified interferon-α2a polypeptide has reduced immunogenicity or a reduced propensity to elicit an immune response as compared to a wild type interferon-α2a polypeptide of SEQ ID NO: 22. In aspects of the above-described polypeptides, the modified interferon-α2a polypeptide may be isolated, synthetic, or recombinant.

In aspects, a modified interferon-α2a polypeptide having interferon-α2a activity comprises an amino acid sequence with at least 60%, 70%, 80%, 90%, or 95% homology to wild type interferon-α2a (SEQ ID NO: 22) and further comprises amino acid substitutions at positions 9, 47, 65, 66, 117, 123, and 128, wherein said substitutions comprise the change of the amino acid of said position to alanine, glycine, or threonine. In aspects, a modified interferon-α2a polypeptide having interferon-α2a activity comprises an amino acid sequence with at least 60%, 70%, 80%, 90%, or 95% homology to wild type interferon-α2a (SEQ ID NO: 22) and further comprises the mutations L9A, F47A, N65A, L66A, L117A, F123A, and L128A. In aspects of the above-described polypeptides, the modified interferon-α2a polypeptide has reduced immunogenicity or a reduced propensity to elicit an immune response as compared to a wild type interferon-α2a polypeptide of SEQ ID NO: 22. In aspects of the above-described polypeptides, the modified interferon-α2a polypeptide may be isolated, synthetic, or recombinant.

In aspects, a modified interferon-α2a polypeptide having interferon-α2a activity comprises an amino acid sequence with at least 60%, 70%, 80%, 90%, or 95% homology to wild type interferon-α2a (SEQ ID NO: 22) and further comprises amino acid substitutions at positions 9, 17, 47, 65, 66, 117, 123, 128, 147, and 157, wherein said substitutions comprise the change of the amino acid of said position to alanine, glycine, or threonine. In aspects, a modified interferon-α2a polypeptide having interferon-α2a activity comprises an amino acid sequence with at least 60%, 70%, 80%, 90%, or 95% homology to wild type interferon-α2a (SEQ ID NO: 22) and further comprises the mutations L9A, L17A, F47A, N65A, L66A, L117A, F123A, L128A, 1147T, and L157A. In aspects of the above-described polypeptides, the modified interferon-α2a polypeptide has reduced immunogenicity or a reduced propensity to elicit an immune response as compared to a wild type interferon-α2a polypeptide of SEQ ID NO: 22. In aspects of the above-described polypeptides, the modified interferon-α2a polypeptide may be isolated, synthetic, or recombinant.

In aspects, a modified interferon-α2a polypeptide having interferon-α2a activity is selected from the group consisting of: SEQ ID NOS: 31-34. In aspects, a modified interferon-α2a polypeptide having interferon-α2a activity is selected from the group consisting of: SEQ ID NO: 32 and SEQ ID NO: 33. In aspects, a modified interferon-α2a polypeptide comprises an amino acid sequence of SEQ ID NO: 31. In aspects, a modified interferon-α2a polypeptide comprises an amino acid sequence of SEQ ID NO: 34. In a preferred embodiment, a modified interferon-α2a polypeptide comprises an amino acid sequence of SEQ ID NO: 32. In aspects, a modified interferon-α2a polypeptide comprises an amino acid sequence of SEQ ID NO: 33. In aspects of the above-described polypeptides, the modified interferon-α2a polypeptides have reduced immunogenicity or a reduced propensity to elicit an immune response as compared to a wild type interferon-α2a polypeptide of SEQ ID NO: 22. In aspects of the above-described polypeptides, the modified interferon-α2a polypeptide may be isolated, synthetic, or recombinant.

In aspects, a modified interferon-α2a polypeptide having interferon-α2a activity, such as the above-described modified interferon-α2a polypeptides, has a relative antiviral activity of between 5% and 95% as compared to a wild type interferon-α2a polypeptide of SEQ ID NO: 22. In aspects, a modified interferon-α2a polypeptide having interferon-α2a activity, such as the above-described modified interferon-α2a polypeptides, has a relative antiviral activity of between 10% and 90% as compared to a wild type interferon-α2a polypeptide of SEQ ID NO: 22. In aspects, a modified interferon-α2a polypeptide having interferon-α2a activity, such as the above-described modified interferon-α2a polypeptides, has a relative antiviral activity of between 20% and 80% as compared to a wild type interferon-α2a polypeptide of SEQ ID NO: 22.

In aspects, the instantly-disclosed modified interferon-α2a polypeptides having interferon-α2a activity, such as the above-described modified interferon-α2a polypeptides, have a percentage antiproliferative biological activity of between 0% and 50%. In aspects, a modified interferon-α2a polypeptide having interferon-α2a activity, such as the above-described modified interferon-α2a polypeptides, has a percentage antiproliferative biological activity of less than 10%. In aspects, a modified interferon-α2a polypeptide having interferon-α2a activity, such as the above-described modified interferon-α2a polypeptides, has a percentage antiproliferative biological activity of less than 5%.

In aspects, the instantly-disclosed modified interferon-α2a polypeptides having interferon-α2a activity, such as the above-described modified interferon-α2a polypeptides, have an apparent plasma clearance rate (Clₐₚₚ) of between 5 mL/h-200 mL/h. In aspects, a modified interferon-α2a polypeptide having interferon-α2a activity, such as the above-described modified interferon-α2a polypeptides, has an apparent plasma clearance rate (Clₐₚₚ) of less than 115 mL/h. In aspects, a modified interferon-α2a polypeptide having interferon-α2a activity, such as the above-described modified interferon-α2a polypeptides, has an apparent plasma clearance rate (Claw) of less than 50 mL/h.

In aspects, the present disclosure provides a polynucleotide or nucleic acid (e.g., DNA, including cDNA, or RNA, including mRNA) encoding a modified interferon-α2a polypeptide having interferon-α2a activity, such as the above-described modified interferon-α2a polypeptides. For example, in aspects, the present disclosure provides a nucleic acid encoding for a modified interferon-α2a polypeptide, wherein the polypeptide comprises an amino acid sequence of SEQ ID NO: 22 and further comprises the following amino acid substitutions: L9A, F47A, L117A, F123A, and L128A. In aspects, the present disclosure provides a nucleic acid encoding for a modified interferon-α2a polypeptide, wherein the polypeptide comprises an amino acid sequence of SEQ ID NO: 22 and further comprises the following amino acid substitutions: L9A, F47A, L117A, F123A, L128A, I147T, and L157A. In aspects, the present disclosure provides a nucleic acid encoding for a modified interferon-α2a polypeptide, wherein the polypeptide comprises an amino acid sequence of SEQ ID NO: 22 and further comprises the following amino acid substitutions: L9A, F47A, N65A, L66A, L117A, F123A, and L128A. In aspects, the present disclosure provides a nucleic acid encoding for a modified interferon-α2a polypeptide, wherein the polypeptide comprises an amino acid sequence of SEQ ID NO: 22 and further comprises the following amino acid substitutions: L9A, L17A, F47A, N65A, L66A, L117A, F123A, L128A, I147T, and L157A.

In aspects, a modified interferon-α2a also comprises the addition of amino acids containing one or more sites of N or O glycosylation, wherein these added amino acids comprise one or more sequences with at least 60%, 70%, 80%, 90%, or 95% homology to APARSPSPSTQPWE or a fragment thereof. In aspects, a modified interferon-α2a as disclosed herein include the addition of one or more of the amino acid sequence APARSPSPSTQPWE (SEQ ID NO: 26) or a fragment thereof. In aspects, said modified interferon-α2a comprises the addition of amino acids containing one or more sites of N or O glycosylation, wherein these added amino acids comprise one or more sequences with at least 70%, 80%, or 90% homology to APARSPSPSTQPWE (SEQ ID NO: 26) or a fragment thereof, and wherein the amino acids at positions 5, 7, 9, and 10 of SEQ ID NO: 26 are not substituted. In aspects, said above described amino acids containing one or more sites of N or O glycosylation (for example, said one or more sequences with at least 60%, 70%, 80%, 90%, or 95% homology to APARSPSPSTQPWE) or a fragment thereof may be added to the N and/or C-terminus of the instantly-disclosed modified interferon-α2a polypeptides. In aspects, said fragment of APARSPSPSTQPWE is at least 5, at least 6, at least 7, at least 8, at least 9 and/or at least 10 amino acids in length. In aspects of the above-described polypeptides, the modified interferon-α2a polypeptides may be isolated, synthetic, or recombinant.

In aspects, a vector or plasmid comprising a nucleic acid of the present disclosure encoding one or more modified interferon-α2a polypeptides of the present disclosure, e.g., but not limited to, a nucleic acid (e.g., DNA or RNA) encoding at least one modified interferon-α2a polypeptide is provided. In aspects, the present disclosure is directed to a cell comprising a vector or plasmid of the present disclosure.

### Modified GMOP-Interferon-α2a Polypeptides and Nucleic Acids

In aspects, a modified interferon-α2 polypeptide of the present disclosure is a modified GMOP-interferon-α2a polypeptide having interferon-α2a activity and a reduced immunogenicity or a reduced propensity to elicit an immune response as compared to a wild type interferon-α2a polypeptide (SEQ ID NO: 22) and/or wild type GMOP-interferon-α2a (SEQ ID NO: 21). In aspects, a modified IFNα-2a polypeptide comprises the addition of amino acids containing one or more sites of N or O glycosylation, wherein these added amino acids comprise one or more sequences with at least 60%, 70%, 80%, 90%, or 95% homology to APARSPSPSTQPWE or a fragment thereof. In aspects, a modified IFNα-2a as disclosed herein includes the addition of one or more of the amino acid sequence APARSPSPSTQPWE (SEQ ID NO: 26) or a fragment thereof.

In aspects, a modified IFNα-2a also comprises the addition of amino acids containing one or more sites of N or O glycosylation, wherein these added amino acids comprise one or more sequences with at least 70%, 80%, or 90% homology to APARSPSPSTQPWE (SEQ ID NO: 26) or a fragment thereof, and wherein the amino acids at positions 5, 7, 9, and 10 of SEQ ID NO: 26 are not substituted. In aspects, said above described amino acids containing one or more sites of N or O glycosylation (for example, said one or more sequences with at least 60%, 70%, 80%, 90%, or 95% homology to APARSPSPSTQPWE or a fragment thereof) may be added to the N and/or C-terminus of the instantly-disclosed modified IFNα-2a polypeptides. In aspects, said fragment of APARSPSPSTQPWE is at least 5, at least 6, at least 7, at least 8, at least 9 and/or at least 10 amino acids in length.

In aspects, a modified GMOP-interferon-α2a polypeptide comprises an amino acid sequence with at least 60%, 70%, 80%, 90%, or 95% homology to wild type GMOP-interferon-α2a (SEQ ID NO: 21) and further comprises one or more amino acid substitutions in any of the positions selected from the set comprised of: 23, 31, 61, 79, 80, 131, 137, 142, 161, and 171. In aspects, a modified GMOP-interferon-α2a polypeptide comprises an amino acid sequence with at least 60%, 70%, 80%, 90%, or 95% homology to wild type GMOP-interferon-α2a (SEQ ID NO: 21) and further comprises one or more amino acid substitutions in any of the positions selected from the set comprised of: 23, 31, 61, 79, 80, 131, 137, 142, 161, and 171, wherein said substitution comprises the change of the amino acid of said position to alanine, glycine, or threonine. In aspects, a modified GMOP-interferon-α2a polypeptide comprises an amino acid sequence with at least 60%, 70%, 80%, 90%, or 95% homology to wild type GMOP-interferon-α2a (SEQ ID NO: 21) and further comprises at least five amino acid substitutions in any of the positions selected from the set comprised of 23, 31, 61, 79, 80, 131, 137, 142, 161, and 171. In aspects, a modified GMOP-interferon-α2a polypeptide comprises an amino acid sequence with at least 60%, 70%, 80%, 90%, or 95% homology to wild type GMOP-interferon-α2a (SEQ ID NO: 21) and further comprises at least five amino acid substitutions in any of the positions selected from the set comprised of: 23, 31, 61, 79, 80, 131, 137, 142, 161, and 171, wherein said substitution comprises the change of the amino acid of said position to alanine, glycine, or threonine. In aspects of the above-described polypeptides, the modified GMOP-interferon-α2a polypeptide may be isolated, synthetic, or recombinant.

In aspects, a modified GMOP-interferon-α2a polypeptide having interferon-α2a activity comprises an amino acid sequence with at least 60%, 70%, 80%, 90%, or 95% homology to wild type GMOP-interferon-α2a (SEQ ID NO: 21) and further comprises one or more amino acid substitutions in any of the positions selected from the set comprised of: 23, 61, 131, 137, and 142, wherein said substitutions comprise the change of the amino acid of said position to alanine, glycine, or threonine. In aspects, a modified GMOP-interferon-α2a polypeptide having interferon-α2a activity comprises an amino acid sequence with at least 60%, 70%, 80%, 90%, or 95% homology to wild type GMOP-interferon-α2a (SEQ ID NO: 21) and further comprises the mutations L23A, F61A, L131A, F137A, and L142A. In aspects of the above-described polypeptides, the modified GMOP-interferon-α2a polypeptide have reduced immunogenicity or a reduced propensity to elicit an immune response as compared to a wild type interferon-α2a polypeptide (SEQ ID NO: 22) and/or wild type GMOP-interferon-α2a (SEQ ID NO: 21). In aspects of the above-described polypeptides, the modified GMOP-interferon-α2a polypeptide may be isolated, synthetic, or recombinant.

In aspects, a modified GMOP-interferon-α2a polypeptide having interferon-α2a activity comprises an amino acid sequence with at least 60%, 70%, 80%, 90%, or 95% homology to wild type GMOP-interferon-α2a (SEQ ID NO: 21) and further comprises one or more amino acid substitutions in any of the positions selected from the set comprised of: 23, 61, 131, 137, 142, 161, and 171, wherein said substitutions comprise the change of the amino acid of said position to alanine, glycine, or threonine. In aspects, a modified GMOP-interferon-α2a polypeptide having interferon-α2a activity comprises an amino acid sequence with at least 60%, 70%, 80%, 90%, or 95% homology to wild type GMOP-interferon-α2a (SEQ ID NO: 21) and further comprises the mutations L23A, F61A, L131A, F137A, L142A, I161T, and L171A. In aspects of the above-described polypeptides, the modified GMOP-interferon-α2a polypeptide have reduced immunogenicity or a reduced propensity to elicit an immune response as compared to a wild type interferon-α2a polypeptide (SEQ ID NO: 22) and/or wild type GMOP-interferon-α2a (SEQ ID NO: 21). In aspects of the above-described polypeptides, the modified GMOP-interferon-α2a polypeptide may be isolated, synthetic, or recombinant.

In aspects, a modified GMOP-interferon-α2a polypeptide having interferon-α2a activity comprises an amino acid sequence with at least 60%, 70%, 80%, 90%, or 95% homology to wild type GMOP-interferon-α2a (SEQ ID NO: 21) and further comprises one or more amino acid substitutions in any of the positions selected from the set comprised of: 23, 61, 79, 80, 131, 137, and 142, wherein said substitutions comprise the change of the amino acid of said position to alanine, glycine, or threonine. In aspects, a modified GMOP-interferon-α2a polypeptide having interferon-α2a activity comprises an amino acid sequence with at least 60%, 70%, 80%, 90%, or 95% homology to wild type GMOP-interferon-α2a (SEQ ID NO: 21) and further comprises the mutations L23A, F61A, N79A, L80A L131A, F137A, and L142A. In aspects of the above-described polypeptides, the modified GMOP-interferon-α2a polypeptide have reduced immunogenicity or a reduced propensity to elicit an immune response as compared to a wild type interferon-α2a polypeptide (SEQ ID NO: 22) and/or wild type GMOP-interferon-α2a (SEQ ID NO: 21). In aspects of the above-described polypeptides, the modified GMOP-interferon-α2a polypeptide may be isolated, synthetic, or recombinant.

In aspects, a modified GMOP-interferon-α2a polypeptide having interferon-α2a activity comprises an amino acid sequence with at least 60%, 70%, 80%, 90%, or 95% homology to wild type GMOP-interferon-α2a (SEQ ID NO: 21) and further comprises one or more amino acid substitutions in any of the positions selected from the set comprised of: 23, 31, 61, 79, 80, 131, 137, 142, 161, and 171, wherein said substitutions comprise the change of the amino acid of said position to alanine, glycine, or threonine. In aspects, a modified GMOP-interferon-α2a polypeptide having interferon-α2a activity comprises an amino acid sequence with at least 70% homology to wild type GMOP-interferon-α2a (SEQ ID NO: 21) and further comprises the mutations L23A, L31A, F61A, N79A, L80A, L131A, F137A, L142A, I161T, and L171A. In aspects of the above-described polypeptides, the modified GMOP-interferon-α2a polypeptide have reduced immunogenicity or a reduced propensity to elicit an immune response as compared to a wild type interferon-α2a polypeptide (SEQ ID NO: 22) and/or wild type GMOP-interferon-α2a (SEQ ID NO: 21). In aspects of the above-described polypeptides, the modified GMOP-interferon-α2a polypeptide may be isolated, synthetic, or recombinant.

In aspects, a modified GMOP-interferon-α2a polypeptide having interferon-α2a activity is selected from the group consisting of: SEQ ID NOS: 27-30. In aspects, a modified GMOP-interferon-α2a polypeptide having interferon-α2a activity is selected from the group consisting of: SEQ ID NO: 28 and SEQ ID NO: 29. In aspects, a modified GMOP-interferon-α2a polypeptide comprises an amino acid sequence of SEQ ID NO: 27. In aspects, a modified GMOP-interferon-α2a polypeptide comprises an amino acid sequence of SEQ ID NO: 30. In aspects, a modified GMOP-interferon-α2a polypeptide comprises an amino acid sequence of SEQ ID NO: 28. In aspects, a modified GMOP-interferon-α2a polypeptide comprises an amino acid sequence of SEQ ID NO: 29. In aspects of the above-described polypeptides, the modified interferon-α2a polypeptides have reduced immunogenicity or a reduced propensity to elicit an immune response as compared to a wild type interferon-α2a polypeptide (SEQ ID NO: 22) and/or wild type GMOP-interferon-α2a (SEQ ID NO: 21). In aspects of the above-described polypeptides, the modified interferon-α2a polypeptide may be isolated, synthetic, or recombinant.

In aspects, a modified GMOP-interferon-α2a polypeptide having interferon-α2a activity, such as the above-described modified GMOP-interferon-α2a polypeptides, has a relative antiviral activity of between 5% and 95% as compared to a wild type interferon-α2a polypeptide (SEQ ID NO: 22) and/or a wild type GMOP-interferon-α2a polypeptide (SEQ ID NO: 21). In aspects, a modified GMOP-interferon-α2a polypeptide having interferon-α2a activity, such as the above-described modified GMOP-interferon-α2a polypeptides, has a relative antiviral activity of between 10% and 90% as compared to a wild type interferon-α2a polypeptide (SEQ ID NO: 22) and/or a wild type GMOP-interferon-α2a polypeptide (SEQ ID NO: 21). In aspects, a modified GMOP-interferon-α2a polypeptide having interferon-α2a activity, such as the above-described modified GMOP-interferon-α2a polypeptides, has a relative antiviral activity of between 20% and 80% as compared to a wild type interferon-α2a polypeptide (SEQ ID NO: 22) and/or a wild type GMOP-interferon-α2a polypeptide (SEQ ID NO: 21).

In aspects, the instantly-disclosed modified GMOP-interferon-α2a polypeptides having interferon-α2a activity, such as the above-described modified GMOP-interferon-α2a polypeptides, have a percentage antiproliferative biological activity of between 0% and 50%. In aspects, a modified GMOP-interferon-α2a polypeptide having interferon-α2a activity, such as the above-described modified GMOP-interferon-α2a polypeptides, has a percentage antiproliferative biological activity of less than 10%. In aspects, a modified GMOP-interferon-α2a polypeptide having interferon-α2a activity, such as the above-described modified GMOP-interferon-α2a polypeptides, has a percentage antiproliferative biological activity of less than 5%.

In aspects, the instantly-disclosed modified GMOP-interferon-α2a polypeptides having interferon-α2a activity, such as the above-described modified GMOP-interferon-α2a polypeptides, have an apparent plasma clearance rate (Clₐpp) of between 5 mL/h-200 mL/h. In aspects, a modified GMOP-interferon-α2a polypeptide having interferon-α2a activity, such as the above-described modified GMOP-interferon-α2a polypeptides, has an apparent plasma clearance rate (Clₐₚₚ) of less than 115 mL/h. In aspects, a modified GMOP-interferon-α2a polypeptide having interferon-α2a activity, such as the above-described modified GMOP-interferon-α2a polypeptides, has an apparent plasma clearance rate (Clₐₚₚ) of less than 50 mL/h.

In aspects, the present disclosure provides a polynucleotide or nucleic acid (e.g., DNA, including cDNA or RNA, including mRNA) encoding a modified GMOP-interferon-α2a polypeptide having interferon-α2a activity, such as the above-described modified GMOP-interferon-α2a polypeptides. For example, in aspects, the present disclosure provides a nucleic acid encoding for a modified GMOP-interferon-α2a polypeptide, wherein the polypeptide comprises an amino acid sequence of SEQ ID NO: 21 and further comprises the following amino acid substitutions: L23A, F61A, L131A, F137A, and L142A. In aspects, the present disclosure provides a nucleic acid encoding for a modified GMOP-interferon-α2a polypeptide, wherein the polypeptide comprises an amino acid sequence of SEQ ID NO: 21 and further comprises the following amino acid substitutions: L23A, F61A, L131A, F137A, L142A, I161T, and L171A. In aspects, the present disclosure provides a nucleic acid encoding for a modified GMOP-interferon-α2a polypeptide, wherein the polypeptide comprises an amino acid sequence of SEQ ID NO: 21 and further comprises the following amino acid substitutions: L23A, F61A, N79A, L80A, L131A, F137A, and L142A. In aspects, the present disclosure provides a nucleic acid encoding for a modified GMOP-interferon-α2a polypeptide, wherein the polypeptide comprises an amino acid sequence of SEQ ID NO: 21 and further comprises the following amino acid substitutions: L23A, L31A, F61A, N79A, L80A, L131A, F137A, L142A, I161T, and L171A.

In aspects, a vector or plasmid comprising a nucleic acid of the invention encoding one or more modified GMOP-interferon-α2a polypeptides of the present disclosure, e.g., but not limited to, a nucleic acid (e.g., DNA or RNA) encoding at least one modified GMOP-interferon-α2a polypeptide is provided. In aspects, the present disclosure is directed to a cell comprising a vector or plasmid of the present disclosure.

### Modified Interferon-α2c Polypeptides and Nucleic Acids

In aspects, a modified interferon-α2 polypeptide of the present disclosure is a modified interferon-α2c polypeptide having interferon-α2c activity and a reduced immunogenicity or a reduced propensity to elicit an immune response as compared to a wild type interferon-α2c polypeptide of SEQ ID NO: 24. In aspects, a modified interferon-α2c polypeptide comprises an amino acid sequence with at least 60%, 70%, 80%, 90%, or 95% and further comprises one or more amino acid substitutions in any of the positions selected from the set comprised of: 9, 17, 47, 65, 66, 117, 123, 128, 147, and 157. In aspects, a modified interferon-α2c polypeptide comprises an amino acid sequence with at least 60%, 70%, 80%, 90%, or 95% homology to wild type interferon-α2c (SEQ ID NO: 24) and further comprises one or more amino acid substitutions in any of the positions selected from the set comprised of: 9, 17, 47, 65, 66, 117, 123, 128, 147, and 157, wherein said substitution comprises the change of the amino acid of said position to alanine, glycine, or threonine. In aspects, a modified interferon-α2c polypeptide comprises an amino acid sequence with at least 60%, 70%, 80%, 90%, or 95% homology to wild type interferon-α2c (SEQ ID NO: 24) and further comprises at least five amino acid substitutions in any of the positions selected from the set comprised of: 9, 17, 47, 65, 66, 117, 123, 128, 147, and 157. In aspects, a modified interferon-α2c polypeptide comprises an amino acid sequence with at least 60%, 70%, 80%, 90%, or 95% homology to wild type interferon-α2c (SEQ ID NO: 24) and further comprises at least five amino acid substitutions in any of the positions selected from the set comprised of: 9, 17, 47, 65, 66, 117, 123, 128, 147, and 157, wherein said substitution comprises the change of the amino acid of said position to alanine, glycine, or threonine. In aspects of the above-described polypeptide, the modified interferon-α2c polypeptide may be isolated, synthetic, or recombinant.

In aspects, a modified interferon-α2c polypeptide having interferon-α2c activity comprises an amino acid sequence with at least 60%, 70%, 80%, 90%, or 95% homology to wild type interferon-α2c (SEQ ID NO: 24) and further comprises amino acid substitutions at positions 9, 47, 117, 123, and 128, wherein said substitutions comprise the change of the amino acid of said position to alanine, glycine, or threonine. In aspects, a modified interferon-α2c polypeptide having interferon-α2c activity comprises an amino acid sequence with at least 60%, 70%, 80%, 90%, or 95% homology to wild type interferon-α2c (SEQ ID NO: 24) and further comprises the mutations L9A, F47A, L117A, F123A, and L128A. In aspects of the above-described polypeptides, the modified interferon-α2c polypeptide have reduced immunogenicity or a reduced propensity to elicit an immune response as compared to a wild type interferon-α2c polypeptide of SEQ ID NO: 24. In aspects of the above-described polypeptides, the modified interferon-α2c polypeptide may be isolated, synthetic, or recombinant.

In aspects, a modified interferon-α2c polypeptide having interferon-α2c activity comprises an amino acid sequence with at least 60%, 70%, 80%, 90%, or 95% homology to wild type interferon-α2c (SEQ ID NO: 24) and further comprises amino acid substitutions at positions 9, 47, 117, 123, 128, 147, and 157, wherein said substitutions comprise the change of the amino acid of said position to alanine, glycine, or threonine. In aspects, a modified interferon-α2c polypeptide having interferon-α2c activity comprises an amino acid sequence with at least 60%, 70%, 80%, 90%, or 95% homology to wild type interferon-α2c (SEQ ID NO: 24) and further comprises the mutations L9A, F47A, L117A, F123A, L128A, I147T, and L157A. In aspects of the above-described polypeptides, the modified interferon-α2c polypeptide have reduced immunogenicity or a reduced propensity to elicit an immune response as compared to a wild type interferon-α2c polypeptide of SEQ ID NO: 24. In aspects of the above-described polypeptides, the modified interferon-α2c polypeptide may be isolated, synthetic, or recombinant.

In aspects, a modified interferon-α2c polypeptide having interferon-α2c activity comprises an amino acid sequence with at least 60%, 70%, 80%, 90%, or 95% homology to wild type interferon-α2c (SEQ ID NO: 24) and further comprises amino acid substitutions at positions 9, 47, 65, 66, 117, 123, and 128, wherein said substitutions comprise the change of the amino acid of said position to alanine, glycine, or threonine. In aspects, a modified interferon-α2c polypeptide having interferon-α2c activity comprises an amino acid sequence with at least 60%, 70%, 80%, 90%, or 95% homology to wild type interferon-α2c (SEQ ID NO: 24) and further comprises the mutations L9A, F47A, N65A, L66A, L117A, F123A, and L128A. In aspects of the above-described polypeptides, the modified interferon-α2c polypeptide have reduced immunogenicity or a reduced propensity to elicit an immune response as compared to a wild type interferon-α2c polypeptide of SEQ ID NO: 24. In aspects of the above-described polypeptides, the modified interferon-α2c polypeptide may be isolated, synthetic, or recombinant.

In aspects, a modified interferon-α2c polypeptide having interferon-α2c activity comprises an amino acid sequence with at least 60%, 70%, 80%, 90%, or 95% homology to wild type interferon-α2c (SEQ ID NO: 24) and further comprises amino acid substitutions at positions 9, 17, 47, 65, 66, 117, 123, 128, 147, and 157, wherein said substitutions comprise the change of the amino acid of said position to alanine, glycine, or threonine. In aspects, a modified interferon-α2c polypeptide having interferon-α2c activity comprises an amino acid sequence with at least 60%, 70%, 80%, 90%, or 95% homology to wild type interferon-α2c (SEQ ID NO: 24) and further comprises the mutations L9A, L17A, F47A, N65A, L66A, L117A, F123A, L128A, I147T, and L157A. In aspects of the above-described polypeptides, the modified interferon-α2c polypeptide have reduced immunogenicity or a reduced propensity to elicit an immune response as compared to a wild type interferon-α2c polypeptide of SEQ ID NO: 24. In aspects of the above-described polypeptides, the modified interferon-α2c polypeptide may be isolated, synthetic, or recombinant.

In aspects, a modified interferon-α2c polypeptide having interferon-α2ca activity is selected from the group consisting of: SEQ ID NOS: 39-42. In aspects, a modified interferon-α2c polypeptide having interferon-α2c activity is selected from the group consisting of: SEQ ID NO: and SEQ ID NO: 41. In aspects, a modified interferon-α2c polypeptide comprises an amino acid sequence of SEQ ID NO: 39. In aspects, a modified interferon-α2c polypeptide comprises an amino acid sequence of SEQ ID NO: 42. In a preferred embodiment, a modified interferon-α2c polypeptide comprises an amino acid sequence of SEQ ID NO: 40. In aspects, a modified interferon-α2c polypeptide comprises an amino acid sequence of SEQ ID NO: 41. In aspects of the above-described polypeptides, the modified interferon-α2c polypeptides have reduced immunogenicity or a reduced propensity to elicit an immune response as compared to a wild type interferon-α2c polypeptide of SEQ ID NO: 24. In aspects of the above-described polypeptides, the modified interferon-α2c polypeptide may be isolated, synthetic, or recombinant.

In aspects, the instantly-disclosed modified interferon-α2c polypeptides having interferon-α2c activity, such as the above-described modified interferon-α2c polypeptides, have a relative antiviral activity of between 5% and 95% as compared to a wild type interferon-α2c polypeptide of SEQ ID NO: 24. In aspects, a modified interferon-α2c polypeptide having interferon-α2c activity, such as the above-described modified interferon-α2c polypeptides, has a relative antiviral activity of between 10% and 90% as compared to a wild type interferon-α2c polypeptide of SEQ ID NO: 24. In aspects, a modified interferon-α2c polypeptide having interferon-α2c activity, such as the above-described modified interferon-α2c polypeptides, has a relative antiviral activity of between 20% and 80% as compared to a wild type interferon-α2c polypeptide of SEQ ID NO: 24.

In aspects, the instantly-disclosed modified interferon-α2c polypeptides having interferon-α2c activity, such as the above-described modified interferon-α2c polypeptides, have a percentage antiproliferative biological activity of between 0% and 50%. In aspects, a modified interferon-α2c polypeptide having interferon-α2c activity, such as the above-described modified interferon-α2c polypeptides, has a percentage antiproliferative biological activity of less than 10%. In aspects, a modified interferon-α2c polypeptide having interferon-α2c activity, such as the above-described modified interferon-α2c polypeptides, has a percentage antiproliferative biological activity of less than 5%.

In aspects, the instantly-disclosed modified interferon-α2c polypeptides having interferon-α2c activity, such as the above-described modified interferon-α2c polypeptides, have an apparent plasma clearance rate (Clₐₚₚ) of between 5 mL/h-200 mL/h. In aspects, a modified interferon-α2c polypeptide having interferon-α2c activity, such as the above-described modified interferon-α2c polypeptides, has an apparent plasma clearance rate (Clₐₚₚ) of less than 115 mL/h. In aspects, a modified interferon-α2c polypeptide having interferon-α2c activity, such as the above-described modified interferon-α2c polypeptides, has an apparent plasma clearance rate (Clₐₚₚ) of less than 50 mL/h.

In aspects, the present disclosure provides a polynucleotide or nucleic acid (e.g., DNA, including cDNA, or RNA, including mRNA) encoding a modified interferon-α2c polypeptide having interferon-α2c activity, such as the above-described modified interferon-α2c polypeptides. For example, in aspects, the present disclosure provides a nucleic acid encoding for a y modified interferon-α2c polypeptide, wherein the polypeptide comprises an amino acid sequence of SEQ ID NO: 24 and further comprises the following amino acid substitutions: L9A, F47A, L117A, F123A, and L128A. In aspects, the present disclosure provides a nucleic acid encoding for a modified interferon-α2c polypeptide, wherein the polypeptide comprises an amino acid sequence of SEQ ID NO: 24 and further comprises the following amino acid substitutions: L9A, F47A, L117A, F123A, L128A, I147T, and L157A. In aspects, the present disclosure provides a nucleic acid encoding for a modified interferon-α2c polypeptide, wherein the polypeptide comprises an amino acid sequence of SEQ ID NO: 24 and further comprises the following amino acid substitutions: L9A, F47A, N65A, L66A, L117A, F123A, and L128A. In aspects, the present disclosure provides a nucleic acid encoding for a modified interferon-α2c polypeptide, wherein the polypeptide comprises an amino acid sequence of SEQ ID NO: 24 and further comprises the following amino acid substitutions: L9A, L17A, F47A, N65A, L66A, L117A, F123A, L128A, I147T, and L157A.

In aspects, a modified interferon-α2c also comprises the addition of amino acids containing one or more sites of N or O glycosylation, wherein these added amino acids comprise one or more sequences with at least 60%, 70%, 80%, 90%, or 95% homology to APARSPSPSTQPWE or a fragment thereof. In aspects, a modified interferon-α2c as disclosed herein include the addition of one or more of the amino acid sequence APARSPSPSTQPWE (SEQ ID NO: 26) or a fragment thereof. In aspects, said modified interferon-α2c comprises the addition of amino acids containing one or more sites of N or O glycosylation, wherein these added amino acids comprise one or more sequences with at least 70%, 80%, or 90% homology to APARSPSPSTQPWE (SEQ ID NO: 26) or a fragment thereof, and wherein the amino acids at positions 5, 7, 9, and 10 of SEQ ID NO: 26 are not substituted. In aspects, said above described amino acids containing one or more sites of N or O glycosylation (for example, said one or more sequences with at least 60%, 70%, 80%, 90%, or 95% homology to APARSPSPSTQPWE) or a fragment thereof may be added to the N and/or C-terminus of the instantly-disclosed modified interferon-α2c polypeptides. In aspects, said fragment of APARSPSPSTQPWE is at least 5, at least 6, at least 7, at least 8, at least 9 and/or at least 10 amino acids in length. In aspects of the above-described polypeptides, the modified interferon-α2c polypeptides may be isolated, synthetic, or recombinant.

In aspects, a vector or plasmid comprising a nucleic acid of the present disclosure encoding one or more modified interferon-α2c polypeptides of the present disclosure, e.g., but not limited to, a nucleic acid (e.g., DNA or RNA) encoding at least one modified interferon-α2c polypeptide is provided. In aspects, the present disclosure is directed to a cell comprising a vector or plasmid of the present disclosure.

### Modified GMOP-Interferon-α2c Polypeptides and Nucleic Acids

In aspects, a modified interferon-α2 polypeptide of the present disclosure, including those described above, is a modified GMOP-interferon-α2c polypeptide having interferon-α2c activity and a reduced immunogenicity or a reduced propensity to elicit an immune response as compared to a wild type interferon-α2c polypeptide (SEQ ID NO: 24) and/or wild type GMOP-interferon-α2c (SEQ ID NO: 23). In aspects, said modified IFN-α2c polypeptides comprise the addition of amino acids containing one or more sites of N or O glycosylation, wherein these added amino acids comprise one or more sequences with at least 60%, 70%, 80%, 90%, or 95% homology to APARSPSPSTQPWE or a fragment thereof. In aspects, a modified IFNα-2c as disclosed herein include the addition of one or more of the amino acid sequence APARSPSPSTQPWE (SEQ ID NO: 26) or a fragment thereof. In aspects, a modified IFNα-2c comprises the addition of amino acids containing one or more sites of N or O glycosylation, wherein these added amino acids comprise one or more sequences with at least 70%, 80%, or 90% homology to APARSPSPSTQPWE (SEQ ID NO: 26) or a fragment thereof, and wherein the amino acids at positions 5, 7, 9, and 10 of SEQ ID NO: 26 are not substituted. In aspects, said above described amino acids containing one or more sites of N or O glycosylation (for example, said one or more sequences with at least 60%, 70%, 80%, 90%, or 95% homology to APARSPSPSTQPWE or a fragment thereof) may be added to the N and/or C-terminus of the instantly-disclosed modified IFN-α2c polypeptides. In aspects, said fragment of APARSPSPSTQPWE is at least 5, at least 6, at least 7, at least 8, at least 9 and/or at least 10 amino acids in length.

In aspects, a modified GMOP-interferon-α2c polypeptide comprises an amino acid sequence with at least 60%, 70%, 80%, 90%, or 95% homology to wild type GMOP-interferon-α2c (SEQ ID NO: 23) and further comprises one or more amino acid substitutions in any of the positions selected from the set comprised of 23, 31, 61, 79, 80, 131, 137, 142, 161, and 171. In aspects, a modified GMOP-interferon-α2c polypeptide comprises an amino acid sequence with at least 60%, 70%, 80%, 90%, or 95% homology to wild type interferon-α2c (SEQ ID NO: 23) and further comprises one or more amino acid substitutions in any of the positions selected from the set comprised of: 23, 31, 61, 79, 80, 131, 137, 142, 161, and 171, wherein said substitution comprises the change of the amino acid of said position to alanine, glycine, or threonine. In aspects, a modified GMOP-interferon-α2c polypeptide comprises an amino acid sequence with at least 60%, 70%, 80%, 90%, or 95% homology to wild type GMOP-interferon-α2c (SEQ ID NO: 23) and further comprises at least five amino acid substitutions in any of the positions selected from the set comprised of: 23, 31, 61, 79, 80, 131, 137, 142, 161, and 171. In aspects, a modified GMOP-interferon-α2c polypeptide comprises an amino acid sequence with at least 60%, 70%, 80%, 90%, or 95% homology to wild type GMOP-interferon-α2c (SEQ ID NO: 23) and further comprises at least five amino acid substitutions in any of the positions selected from the set comprised of: 23, 31, 61, 79, 80, 131, 137, 142, 161, and 171, wherein said substitution comprises the change of the amino acid of said position to alanine, glycine, or threonine. In aspects of the above-described polypeptide, the modified GMOP-interferon-α2c polypeptide may be isolated, synthetic, or recombinant.

In aspects, a modified GMOP-interferon-α2c polypeptide having interferon-α2c activity comprises an amino acid sequence with at least 60%, 70%, 80%, 90%, or 95% homology to wild type GMOP-interferon-α2c (SEQ ID NO: 23) and further comprises one or more amino acid substitutions in any of the positions selected from the set comprised of: 23, 61, 131, 137, and 142, wherein said substitutions comprise the change of the amino acid of said position to alanine, glycine, or threonine. In aspects, a modified GMOP-interferon-α2c polypeptide having interferon-α2c activity comprises an amino acid sequence with at least 60%, 70%, 80%, 90%, or 95% homology to wild type GMOP-interferon-α2c (SEQ ID NO: 23) and further comprises the mutations L23A, F61A, L131A, F137A, and L142A. In aspects of the above-described polypeptides, the modified GMOP-interferon-α2c polypeptide have reduced immunogenicity or a reduced propensity to elicit an immune response as compared to a wild type interferon-α2c polypeptide (SEQ ID NO: 24) and/or wild type GMOP-interferon-α2c (SEQ ID NO: 23). In aspects of the above-described polypeptides, the modified interferon-α2c polypeptide may be isolated, synthetic, or recombinant.

In aspects, a modified GMOP-interferon-α2c polypeptide having interferon-α2c activity comprises an amino acid sequence with at least 60%, 70%, 80%, 90%, or 95% homology to wild type GMOP-interferon-α2c (SEQ ID NO: 23) and further comprises one or more amino acid substitutions in any of the positions selected from the set comprised of: 23, 61, 131, 137, 142, 161, and 171, wherein said substitutions comprise the change of the amino acid of said position to alanine, glycine, or threonine. In aspects, a modified GMOP-interferon-α2c polypeptide having interferon-α2c activity comprises an amino acid sequence with at least 60%, 70%, 80%, 90%, or 95% homology to wild type GMOP-interferon-α2c (SEQ ID NO: 23) and further comprises the mutations L23A, F61A, L131A, F137A, L142A, I161T, and L171A. In aspects of the above-described polypeptides, the modified GMOP-interferon-α2c polypeptide have reduced immunogenicity or a reduced propensity to elicit an immune response as compared to a wild type interferon-α2c polypeptide (SEQ ID NO: 24) and/or wild type GMOP-interferon-α2c (SEQ ID NO: 23). In aspects of the above-described polypeptides, the modified interferon-α2c polypeptide may be isolated, synthetic, or recombinant.

In aspects, a modified GMOP-interferon-α2c polypeptide having interferon-α2c activity comprises an amino acid sequence with at least 60%, 70%, 80%, 90%, or 95% homology to wild type GMOP-interferon-α2c (SEQ ID NO: 23) and further comprises one or more amino acid substitutions in any of the positions selected from the set comprised of: 23, 61, 79, 80, 131, 137, and 142, wherein said substitutions comprise the change of the amino acid of said position to alanine, glycine, or threonine. In aspects, a modified GMOP-interferon-α2c polypeptide having interferon-α2c activity comprises an amino acid sequence with at least 60%, 70%, 80%, 90%, or 95% homology to wild type GMOP-interferon-α2c (SEQ ID NO: 23) and further comprises the mutations L23A, F61A, N79A, L80A L131A, F137A, and L142A. In aspects of the above-described polypeptides, the modified GMOP-interferon-α2c polypeptide have reduced immunogenicity or a reduced propensity to elicit an immune response as compared to a wild type interferon-α2c polypeptide (SEQ ID NO: 24) and/or wild type GMOP-interferon-α2c (SEQ ID NO: 23). In aspects of the above-described polypeptides, the modified interferon-α2c polypeptide may be isolated, synthetic, or recombinant.

In aspects, a modified GMOP-interferon-α2c polypeptide having interferon-α2c activity comprises an amino acid sequence with at least 60%, 70%, 80%, 90%, or 95% homology to wild type GMOP-interferon-α2c (SEQ ID NO: 23) and further comprises one or more amino acid substitutions in any of the positions selected from the set comprised of: 23, 31, 61, 79, 80, 131, 137, 142, 161, and 171 wherein said substitutions comprise the change of the amino acid of said position to alanine, glycine, or threonine. In aspects, a modified GMOP-interferon-α2c polypeptide having interferon-α2c activity comprises an amino acid sequence with at least 60%, 70%, 80%, 90%, or 95% homology to wild type GMOP-interferon-α2c (SEQ ID NO: 23) and further comprises the mutations L23A, L31A, F61A, N79A, L80A L131A, F137A, L142A, I161T, and L171A. In aspects of the above-described polypeptides, the modified GMOP-interferon-α2c polypeptide have reduced immunogenicity or a reduced propensity to elicit an immune response as compared to a wild type interferon-α2c polypeptide (SEQ ID NO: 24) and/or wild type GMOP-interferon-α2c (SEQ ID NO: 23). In aspects of the above-described polypeptides, the modified interferon-α2c polypeptide may be isolated, synthetic, or recombinant.

In aspects, a modified GMOP-interferon-α2c polypeptide having interferon-α2c activity is selected from the group consisting of: SEQ ID NOS: 35-38. In aspects, a modified GMOP-interferon-α2c polypeptide having interferon-α2c activity is selected from the group consisting of: SEQ ID NO: 36 and SEQ ID NO: 37. In aspects, a modified GMOP-interferon-α2c polypeptide comprises an amino acid sequence of SEQ ID NO: 35. In aspects, a modified GMOP-interferon-α2c polypeptide comprises an amino acid sequence of SEQ ID NO: 38. In aspects, a modified GMOP-interferon-α2c polypeptide comprises an amino acid sequence of SEQ ID NO: 36. In aspects, a modified GMOP-interferon-α2c polypeptide comprises an amino acid sequence of SEQ ID NO: 37. In aspects of the above-described polypeptides, the modified interferon-α2c polypeptides have reduced immunogenicity or a reduced propensity to elicit an immune response as compared to a wild type interferon-α2c polypeptide (SEQ ID NO: 24) and/or wild type GMOP-interferon-α2c (SEQ ID NO:23). In aspects of the above-described polypeptides, the modified interferon-α2c polypeptide may be isolated, synthetic, or recombinant.

In aspects, a modified GMOP-interferon-α2c polypeptide having interferon-α2c activity, such as the above-described modified GMOP-interferon-α2c polypeptides, have a relative antiviral activity of between 5% and 95% as compared to a wild type interferon-α2c polypeptide (SEQ ID NO: 24) and/or wild type GMOP-interferon-α2c (SEQ ID NO: 23). In aspects, a modified GMOP-interferon-α2c polypeptide having interferon-α2c activity, such as the above-described modified GMOP-interferon-α2c polypeptides, has a relative antiviral activity of between 10% and 90% as compared to a wild type interferon-α2c polypeptide (SEQ ID NO: 24) and/or wild type GMOP-interferon-α2c (SEQ ID NO: 23). In aspects, a modified GMOP-interferon-α2c polypeptide having interferon-α2c activity, such as the above-described modified GMOP-interferon-α2c polypeptides, has a relative antiviral activity of between 20% and 80% as compared to a wild type interferon-α2c polypeptide (SEQ ID NO: 24) and/or wild type GMOP-interferon-α2c (SEQ ID NO: 23).

In aspects, the instantly-disclosed modified GMOP-interferon-α2c polypeptides having interferon-α2c activity, such as the above-described modified GMOP-interferon-α2c polypeptides, have a percentage antiproliferative biological activity of between 0% and 50%. In aspects, a modified GMOP-interferon-α2c polypeptide having interferon-α2c activity, such as the above-described modified GMOP-interferon-α2c polypeptides, has a percentage antiproliferative biological activity of less than 10%. In aspects, a modified GMOP-interferon-α2c polypeptide having interferon-α2c activity, such as the above-described modified GMOP-interferon-α2c polypeptides, has a percentage antiproliferative biological activity of less than 5%.

In aspects, the instantly-disclosed modified GMOP-interferon-α2c polypeptides having interferon-α2c activity, such as the above-described modified GMOP-interferon-α2c polypeptides, have an apparent plasma clearance rate (Clₐₚₚ) of between 5 mL/h-200 mL/h. In aspects, a modified GMOP-interferon-α2c polypeptide having interferon-α2c activity, such as the above-described modified GMOP-interferon-α2c polypeptides, has an apparent plasma clearance rate (Clₐₚₚ) of less than 115 mL/h. In aspects, a modified GMOP-interferon-α2c polypeptide having interferon-α2c activity, such as the above-described modified GMOP-interferon-α2c polypeptides, has an apparent plasma clearance rate (C1a) of less than 50 mL/h.

In aspects, the present disclosure provides a polynucleotide or nucleic acid (e.g., DNA, including cDNA or RNA, including mRNA) encoding a modified GMOP-interferon-α2c polypeptide having interferon-α2c activity, such as the above-described modified GMOP-interferon-α2c polypeptides. For example, in aspects, the present disclosure provides a nucleic acid encoding for a modified GMOP-interferon-α2c polypeptide, wherein the polypeptide comprises an amino acid sequence of SEQ ID NO: 23 and further comprises the following amino acid substitutions: L23A, F61A, L131A, F137A, and L142A. In aspects, the present disclosure provides a nucleic acid encoding for a modified GMOP-interferon-α2c polypeptide, wherein the polypeptide comprises an amino acid sequence of SEQ ID NO: 23 and further comprises the following amino acid substitutions: L23A, F61A, L131A, F137A, L142A, I161T, and L171A. In aspects, the present disclosure provides a nucleic acid encoding for a modified GMOP-interferon-α2c polypeptide, wherein the polypeptide comprises an amino acid sequence of SEQ ID NO: 23 and further comprises the following amino acid substitutions: L23A, F61A, N79A, L80A L131A, F137A, and L142A. In aspects, the present disclosure provides a nucleic acid encoding for a modified GMOP-interferon-α2c polypeptide, wherein the polypeptide comprises an amino acid sequence of SEQ ID NO: 23 and further comprises the following amino acid substitutions: L23A, L31A, F61A, N79A, L80A L131A, F137A, L142A, I161T, and L171A.

In aspects, a vector or plasmid comprising a nucleic acid of the invention encoding one or more modified GMOP-interferon-α2c polypeptides of the present disclosure, e.g., but not limited to, a nucleic acid (e.g., DNA or RNA) encoding at least one modified GMOP-interferon-α2c polypeptide is provided. In aspects, the present disclosure is directed to a cell comprising a vector or plasmid of the present disclosure.

In aspects, a modified interferon-α2 polypeptide as described herein is joined to or linked to (e.g., fused in-frame, chemically-linked, or otherwise bound) a heterologous polypeptide. With respect to the one or more modified interferon-α2 polypeptides of the instant disclosure, the term “heterologous polypeptide” is intended to mean that the one or more modified interferon-α2 polypeptides of the instant disclosure are heterologous to, or not included naturally, in the heterologous polypeptide. In aspects, one or more of the instantly-modified interferon-α2 polypeptides may be added to the C-terminus (with or without the use of linkers, as is known in the art), and/or added to the N-terminus (with or without the use of linkers, as is known in the art) of the heterologous polypeptide.

The present disclosure also provides chimeric or fusion polypeptides (which in aspects may be isolated, synthetic, or recombinant) wherein one or more of the instantly disclosed modified interferon-α2 polypeptides is a part thereof. In aspects, the one or more modified interferon-α2 polypeptides of the present disclosure can be joined or linked to (e.g., fused in-frame, chemically-linked, or otherwise bound) a small molecule, drug, or drag fragment, for example, but not limited to, a drug or drug fragment that is binds with high affinity to defined receptors.

As used herein, two polypeptides (or a region of the polypeptides) are substantially homologous or identical when the amino acid sequences have a certain percentage or more identity, e.g., at least about 45%, 50%, 60%, 65%, 70%, 75%, 80%, 85%, 90%, 91%, 92%, 93%, 94%, 95%, 96%, 97%, 98%, or 99% identity, typically at least about 70-75%, more typically at least about 80-85%, more typically greater than about 90%, and more typically greater than 95% or more homologous or identical. Percent homology can be determined as is known in the art. For example, to determine the percent homology or identity of two amino acid sequences, or of two nucleic acid sequences, the sequences are aligned for optimal comparison purposes (e.g., gaps can be introduced in the sequence of one polypeptide or nucleic acid molecule for optimal alignment with the other polypeptide or nucleic acid molecule). The amino acid residues or nucleotides at corresponding amino acid positions or nucleotide positions are then compared. When a position in the first sequence is occupied by the same amino acid residue as the corresponding position in the second sequence, then the molecules are identical at that position (as used herein amino acid “identity” is equivalent to amino acid “homology”). As is known in the art, the percent identity between the two sequences is a function of the number of identical positions shared by the sequences, taking into account the number of gaps, and the length of each gap, which need to be introduced for optimal alignment of the two sequences. Sequence homology for polypeptides is typically measured using sequence analysis software.

In aspects, the present disclosure also encompasses polypeptides (e.g., modified interferon-α2 polypeptides and modified interferon-α2 compositions as disclosed herein) having a lower degree of identity but having sufficient similarity so as to perform one or more of the same functions performed by a polypeptide encoded by a nucleic acid molecule of the invention. Similarity is determined by conserved amino acid substitution. Such substitutions are those that substitute a given amino acid in a polypeptide by another amino acid of like characteristics. Conservative substitutions are likely to be phenotypically silent. Typically seen as conservative substitutions are the replacements, one for another, among the aliphatic amino acids Ala, Val, Leu, Met, and lie; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Trp, and Tyr. Guidance concerning which amino acid changes are likely to be phenotypically silent are found (Bowie J U et al., (1990), Science, 247(4948):130610, which is herein incorporated by reference in its entirety). For example, amino acid sequences having the function of an interferon can be identified by performing a protein-protein BLAST (blastp) search of the non-redundant protein sequences (nr) database using the amino acid sequences of these proteins as query. The search can be conducted on the National Center for Biotechnology Information (NCBI) website (http.//blast.ncbi.nlm.nih.gov) using default parameters.

Fragments and variants of the disclosed modified IFNα-2 polypeptides and polynucleotides are also encompassed by the present disclosure. “Fragment” is intended to mean a portion of the polypeptide or polynucleotide. Fragments of a polypeptide or a nucleotide sequence as disclosed herein may encode polypeptide fragments that retain the biological activity of the polypeptides of the instant disclosure, and hence have retain interferon-α2 activity (e.g., antiviral biological activity) with reduced immunogenicity as compared to wild-type interferon-α2. In aspects, the present disclosure also encompasses fragments of the variants of the polypeptides and polynucleotides described herein.

In aspects, a variant polypeptide (e.g., a variant of a modified interferon-α2 polypeptide of the present disclosure) can differ in amino acid sequence by one or more substitutions, deletions, insertions, inversions, fusions, and truncations or a combination of any of these.

Variant polypeptides can be fully functional (e.g., retain interferon-α2 activity, such as antiviral biological activity) or can lack function in one or more activities. Fully functional variants typically contain only conservative variation or variation in non-critical residues or in non-critical regions. Functional variants can also contain substitution of similar amino acids that result in no change or an insignificant change in function (e.g., retain antiviral biological activity with reduced immunogenicity). Alternatively, such substitutions can positively or negatively affect function to some degree. Non-functional variants typically contain one or more non-conservative amino acid substitutions, deletions, insertions, inversions, or truncation or a substitution, insertion, inversion, or deletion in a critical residue or critical region. In aspects, a modified interferon-α2 polypeptide of the instant disclosure can differ in amino acid sequence by one or more substitutions, deletions, insertions, inversions, fusions, and truncations or a combination of any of these, provided said variants retain biological activity (e.g., IFNα-2 activity, such an antiviral activity) and have reduced immunogenicity (as compared to wild-type interferon-α2).

In aspects, fully functional variants of modified interferon-α2 do not contain mutations at one or more critical residues or regions. In aspects, said one or more critical residues of modified interferon-α2 that should not be mutated include: residues involved in biological activity, residues of functional hotspots that are heavily conserved between various wild type interferon alleles (such as between species), residues implicated in binding to the interferon's natural receptor, residues involved in structural interactions that are important to the structural integrity of the natural interferon, residues engaged in disulfide bonds of the natural interferon (e.g., intramolecular disulfide bonds that occur in the natural interferon upon proper folding in its natural environment in vivo), and/or residues that are the site of glycosylation in the natural, wild type interferon (including N-glycosylation sites and O-glycosylation sites).

In aspects, the instantly-disclosed modified IFNα-2 polypeptides, including fully functional variants of disclosed modified interferon-α2, do not contain mutations at one or more critical residues or regions, wherein said one or more critical residues or regions are selected from the group comprising: residues of functional hotspots, residues that are heavily conserved between various wild type interferon alleles (such as between species), residues engaged in disulfide bonds of the natural interferon (e.g., intramolecular disulfide bonds that occur in the natural interferon upon proper folding in its natural environment in vivo), and/or residues that are the site of glycosylation in the natural, wild type interferon (including N-glycosylation sites and 0-glycosylation sites).

In aspects, amino acid residues which are not believed to be essential for the functioning of the instantly-disclosed polypeptides, including fully functional variants of disclosed modified interferon-α2 (e.g., IFNα-2b variants, IFNα-2a variants, IFNα-2c variants, GMOP-IFNα-2b variants, GMOP-IFNα-2a variants, and GMOP-IFNα-2c variants), may be substituted either conservatively or non-conservatively, and such amino acid substitutions would likely not significantly diminish the functional properties of the polypeptides. In aspects, amino acid residues which are believed to be essential for the functioning of the instantly-disclosed polypeptides, including fully functional variants of disclosed modified interferon-α2 (e.g., IFNα-2b variants, IFNα-2a variants, IFNα-2c variants, GMOP-IFNα-2b variants, GMOP-IFNα-2a variants, and GMOP-IFNα-2c variants), may be not be substituted either conservatively or non-conservatively, as such amino acid substitutions would likely significantly diminish the functional properties of the polypeptides. In aspects, the instantly-disclosed modified IFNα-2 polypeptides, including fully functional variants of disclosed modified interferon-α2 (e.g., IFNα-2b variants, IFNα-2a variants, IFNα-2c variants, GMOP-IFNα-2b variants, GMOP-IFNα-2a variants, and GMOP-IFNα-2c variants), do not contain mutations (either conservative or nonconservative substitutions) at one or more critical residues or regions of WT natural human IFN-α2. In aspects, said one or more critical residues or regions of WT natural human IFN-α2 are selected from the group comprising: residues involved in biological activity, residues of functional hotspots, residues that are heavily conserved between various wild type interferon alleles (such as between species), residues implicated in binding to the interferon's natural receptor, residues involved in structural interactions that are important to the structural integrity of the natural interferon, residues engaged in disulfide bonds of the natural interferon (e.g., intramolecular disulfide bonds that occur in the natural interferon upon proper folding in its natural environment in vivo), and/or residues that are the site of glycosylation in the natural, wild type interferon (including N-glycosylation sites and O-glycosylation sites). In aspects, said one or more critical residues or regions of WT natural hIFN-α2 involved in the biological activity of hIFN-α2 are selected from the group consisting of 22, 26, 27, 30, 31, 33, 34, 36, 68, 79, 85, 120, 121, 122, 124, 129, 131, 132, 144, and 146, and most conservative and nonconservative amino acid substitutions for such amino acid residues will likely diminish the functional properties (e.g., IFNα-2 activity, including antiviral activity) of the polypeptides. In aspects, said one or more critical residues or regions of WT natural hIFN-α2 that are functional hotspots are selected from the group consisting of 30, 33, 144, 145, 148, and 149, and most conservative and nonconservative amino acid substitutions for such amino acid residues will likely diminish the functional properties (e.g., IFNα-2 activity, including antiviral activity) of the polypeptides. In aspects, said one or more critical residues or regions of WT natural hIFN-α2 that are heavily conserved in between various wild type IFN-α2 alleles (such as between species) are selected from the group consisting of: 91, 122, 150, and 154 (and may additionally comprise: 30, 33, 144, 145, 148, and 149), and most conservative and nonconservative amino acid substitutions for such amino acid residues will likely diminish the functional properties (e.g., IFNα-2 activity, including antiviral activity) of the polypeptides. In aspects, said one or more critical residues or regions of WT natural hIFN-α2 that are implicated in binding to the hIFN-α2's natural receptor are selected from the group consisting of: 5, 6, 12, 13, 15, 16, 19, 20, 22, 26, 27, 30-37, 39-41, 46, 68, 76, 77, 79, 80, 82, 83, 85, 86, 89, 90, 93, 94, 97, 118, 120, 121, 124, 125, 127, 131-136, 144-146, 148, 149, 15, and 153, and most conservative and nonconservative amino acid substitutions for such amino acid residues will likely diminish the functional properties (e.g., IFNα-2 activity, including antiviral activity) of the polypeptides. In aspects, said one or more critical residues or regions of WT natural hIFN-α2 that are involved in structural interactions that are important to the structural integrity of the hIFN-α2 are selected from the group consisting of: 33, 34, 35, 36, 38, 40, 41, 42, 43, 44, 45, 91, 114, 115, 118, 121,122, 125, 132, 150, and 154, and most conservative and nonconservative amino acid substitutions for such amino acid residues will likely diminish the functional properties (e.g., IFNα-2 activity, including antiviral activity) of the polypeptides. In aspects, said one or more critical residues or regions of WT natural hIFN-α2 that are involved in structural interactions that are important to the structural integrity of the hIFN-α2 are selected from the group consisting of: 36, 41, 42, 91, 122, 129, 150, and 154, and most conservative and nonconservative amino acid substitutions for such amino acid residues will likely diminish the functional properties (e.g., IFNα-2 activity, including antiviral activity) of the polypeptides. In aspects, said one or more critical residues or regions of WT natural hIFN-α2 that are engaged in disulfide bonds of the natural hIFN-α2 (e.g., intramolecular disulfide bonds that occur in the hIFN-α2 upon proper folding in its natural environment in vivo) are selected from the group consisting of: 1, 29, 98, and 138, and most conservative and nonconservative amino acid substitutions for such amino acid residues will likely diminish the functional properties (e.g., IFNα-2 activity, including antiviral activity) of the polypeptides. In aspects, said one or more critical residues or regions of WT natural hIFN-α2 that that are the site of glycosylation in the natural, wild type hIFN-α2 (including N-glycosylation sites and O-glycosylation sites) are selected from the group consisting of: 106. It is believed that the instantly-disclosed polypeptides having the described modifications/substitutions would confer the desired activity (e.g., the IFNα-2 activity, including antiviral activity). Stated another way, it is believed that the amino acid substitutions described herein would not significantly diminish the functional properties of the instantly-disclosed polypeptides.

In aspects, a modified interferon-α2 (e.g., fully functional variants of disclosed modified interferon-α2 (e.g., IFNα-2b variants, IFNα-2a variants, IFNα-2c variants, GMOP-IFNα-2b variants, GMOP-IFNα-2a variants, and GMOP-IFNα-2c variants)) does not contain mutations (e.g., amino acid substitutions) at one or more amino acids, wherein said one or more amino acids occupy positions selected from the group consisting of the following positions in hIFN-α2: 4, 23, 70, and 77. In aspects, a modified interferon-α2 (e.g., fully functional variants of disclosed modified interferon-α2 (e.g., IFNα-2b variants, IFNα-2a variants, and IFNα-2c variants) does not contain the substitution of an amino acid for an Asn residue at one or more amino acids, wherein said one or more amino acids occupy positions selected from the group consisting of the following positions in hIFN-α2: 4, 23, 70, and 77. In aspects, a modified interferon-α2 does not contain one or more amino acid substitutions at the amino acid positions selected from the group consisting of the following positions in hIFN-α2: 4, 23, 70, and 77.

In aspects, a modified GMOP-interferon-α2 (e.g., fully functional variants of disclosed modified GMOP-IFNα-2b variants, GMOP-IFNα-2a variants, and GMOP-IFNα-2c variants) does not contain mutations (e.g., amino acid substitutions) at one or more amino acids, wherein said one or more amino acids occupy positions selected from the group consisting of the following positions in GMOP-hIFN-α2: 18, 37, 84, and 91. In aspects, a modified interferon-α2 (e.g., fully functional variants of disclosed modified GMOP-IFNα-2b variants, GMOP-IFNα-2a variants, and GMOP-IFNα-2c variants)) does not contain the substitution of an amino acid for an Asn residue at one or more amino acids, wherein said one or more unsubstituted amino acids occupy positions selected from the group consisting of the following positions in GMOP-hIFN-α2: 18, 37, 84, and 91. In aspects, a modified interferon-α2 (e.g., fully functional variants of disclosed modified GMOP-IFNα-2b variants, GMOP-IFNα-2a variants, and GMOP-IFNα-2c variants) does not contain one or more amino acid substitutions at the amino acid positions selected from the group consisting of the following positions in GMOP-hIFN-α2: 18, 37, 84, and 91.

In aspects, a modified interferon-α2 of the present disclosure can include allelic or sequence variants (“mutants”) or analogs thereof, or can include chemical modifications (e.g., pegylation, glycosylation). In aspects, a modified interferon-α2 polypeptide as described herein is hyperglycosylated. In aspects, a modified interferon-α2 retains the same functions performed by an interferon polypeptide encoded by a nucleic acid molecule of the present disclosure, particularly maintained biological activity and reduced immunogenicity. In aspects, a modified interferon-α2 can provide for high relative antiviral activity. In aspects, a modified interferon-α2 can lead to reduced immunogenicity. In aspects, a modified interferon-α2 can lead to low antiproliferative biological activity. In aspects, a modified interferon-α2 can lead to improved pharmacokinetic profile. In aspects, a modified interferon-α2 can lead to improvements in protein synthesis and purification of the modified interferon-α2.

The polypeptides of the instant disclosure may be altered in various ways including amino acid substitutions, deletions, truncations, and insertions. Methods for such manipulations are generally known in the art. For example, amino acid sequence variants and fragments of the instantly-disclosed polypeptides can be prepared by mutations in the DNA. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192: Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal. Typically seen as conservative substitutions are the replacements, one for another, among the aliphatic amino acids Ala, Val, Leu and lie; interchange of the hydroxyl residues Ser and Thr, exchange of the acidic residues Asp and Glu, substitution between the amide residues Asn and Gin, exchange of the basic residues Lys and Arg and replacements among the aromatic residues Phe and Tyr. Guidance concerning which amino acid changes are likely to be phenotypically silent are found (Bowie J U et al., (1990), Science, 247(4948):130610, which is herein incorporated by reference in its entirety). For the purposes of the present disclosure, polypeptides can include, for example, modified forms of naturally occurring amino acids such as D-stereoisomers, non-naturally occurring amino acids; amino acid analogs; and mimetics.

It should be clarified that the modifications/substitutions presented by the deimmunized variants of GMOP-IFNα-2 correspond to those amino acids not involved in the biological structure or function of cytokine. That is, these mutations can be performed on any of the following interferon variants: IFN-α2b, GMOP-IFN-α2b, or any other variant of IFN-α2 (including IFN-α2a, GMOP-IFN-α2A, IFN-α2c, and GMOP-IFN-α2c).

The manner of producing the modified interferon-α2 polypeptides of the present disclosure will vary widely, depending upon the nature of the various elements comprising the molecule. For example, an isolated polypeptide (e.g., an isolated modified interferon-α2 polypeptide) can be purified from cells that naturally express it, purified from cells that have been altered to express it (recombinant), or synthesized using known protein synthesis methods. The synthetic procedures may be selected so as to be simple, provide for high yields, and allow for a highly purified stable product. For example, polypeptides of the instant disclosure can be produced either from a nucleic acid disclosed herein, or by the use of standard molecular biology techniques, such as recombinant techniques, mutagenesis, or other known means in the art. An isolated polypeptide can be purified from cells that naturally express it, purified from cells that have been altered to express it (recombinant), or synthesized using known protein synthesis techniques. In aspects, a polypeptide of the instant disclosure is produced by recombinant DNA or RNA techniques. In aspects, a polypeptide of the instant disclosure can be produced by expression of a recombinant nucleic acid of the instant disclosure in an appropriate host cell. For example, a nucleic acid molecule encoding the polypeptide is cloned into an expression cassette or expression vector, the expression cassette or expression vector introduced into a host cell and the polypeptide expressed in the host cell. The polypeptide can then be isolated from the cells by an appropriate purification scheme using standard protein purification techniques. Alternatively a polypeptide can be produced by a combination of ex vivo procedures, such as protease digestion and purification. Further, polypeptides of the instant disclosure can be produced using site-directed mutagenesis techniques, or other mutagenesis techniques known in the art (see e.g., James A. Brannigan and Anthony J. Wilkinson., 2002, Protein engineering 20 years on. Nature Reviews Molecular Cell Biology 3, 964-970; Turanli-Yildiz B. et al., 2012, Protein Engineering Methods and Applications, intechopen.com, which are herein incorporated by reference in their entirety).

In aspects, the present disclosure is also directed to a method of synthesizing modified interferon-α2 (e.g., including the modified IFNα-2b polypeptides, the modified IFNα-2a polypeptides, modified IFNα-2c polypeptides, modified GMOP-IFNα-2b polypeptides, modified GMOP-IFNα-2a polypeptides, and modified GMOP-IFNα-2c polypeptides). The instantly-disclosed modified interferon-α2 polypeptides with the improved properties (e.g., with reduced immunogenicity) can be created through genetic modification in one of a variety of ways that are described herein. The term “modified interferon-α2” as used herein, may refer to the group of instantly disclosed modified interferon-α2 having an intentionally altered amino acid sequence, i.e., a “non-wild type” amino acid sequence, or to a microbial organism (depending upon placement of either term as an adjective) having a genome that has been intentionally altered as to (at least) the specific, modified interferon-α2 molecules described herein, or both. Such alterations may be accomplished via recombinant technology, wherein one or more genes are transferred from a second, different microbial organism into a target microbial organism. Recombinant technology can be accomplished using fully synthetic DNA that is transferred to the target microbial organism using conventional methods. Such alterations may also be accomplished via engineered technology, wherein the nucleic acids within the target microbial organism are altered, generally via site-directed mutagenesis, resulting in the conversion of at least one nucleic acid to a different nucleic acid and therefore modification of one or more enzymes. Combinations of any of the above methods and those described throughout the application may also be employed. Thus, it will be understood that the instantly disclosed modified interferon-α2 molecule can be produced either in vivo, i.e., by a genetically modified microorganism, or in vitro.

In aspects, the present disclosure provides a method for generating said amino acid substitutions to reduce immunogenicity. Said method comprises the generation of point mutations in the nucleotide sequence of the gene encoding the human natural interferon (e.g., natural IFN-α2, natural GMOP-IFN-α2), by means of a site-directed mutagenesis technique in said gene. The method comprises the following steps: 1. cloning a gene encoding natural human interferon (e.g., natural IFN-α2, natural GMOP-IFN-α2) in a suitable plasmid; 2. generating mutations required for producing the modified interferon-α2 of the present disclosure using a site-directed mutagenesis technique; and 3. cloning the modified gene from step 2, into a suitable expression vector. In aspects, the expression vector is selected from the group of vectors capable of carrying the gene of the present disclosure and further containing the necessary elements for expressing the gene of interest in eukaryotic cells.

In aspects, the site-directed mutagenesis technique of the present disclosure involves the use of oligonucleotides specifically designed to that end. This technique comprises two stages. In the first stage, two PCR reactions are carried out separately using oligonucleotides that hybridize to the terminal ends of the fragment cloned into a suitable vector, and oligonucleotides carrying a point mutation corresponding to an amino acid substitution that reduces immunogenicity (as described here) which hybridize to the internal region of the gene where the mutation is to be introduced. A reaction mixture is obtained in tube a using a reverse external oligonucleotide and the direct oligonucleotide mut a. Another reaction mixture is obtained in tube b with a direct external oligonucleotide and the reverse oligonucleotide mut b. PCR products from both reactions are purified by agarose gel electrophoresis and used as a template for the second stage. This second stage comprises a second PCR reaction using direct and reverse external oligonucleotides. The first three cycles are carried out without the addition of primers to allow hybridization and elongation of the complete product (fill in) and finally these are added for the amplification.

In aspects, to obtain more than one amino acid substitution sites that reduce immunogenicity within a modified interferon-α2 of the instant disclosure, said modified interferon-α2 is constructed sequentially as follows: first, a modified interferon-α2 with amino acid substitution site is generated, using a site-directed mutagenesis technique, and then said modified interferon-α2 is used as a starting template for generating a new amino acid substitution site.

In aspects, the present disclosure is directed to a method for producing a modified interferon-α2 comprising the steps of: a) transforming or transfecting a prokaryotic cell with a suitable prokaryotic expression vector containing the gene encoding a modified interferon-α2; b) selecting a clone expressing the polypeptide of the modified interferon-α2; c) culturing said clone in a suitable culture medium, d) purifying the product, e) glycosylating in vitro the modified interferon-α2 polypeptide expressed by the clone of step c); and f) purifying the modified interferon-α2. In aspects, the glycosylation in step e) of said method is a hyperglycosylation of the modified interferon-α2 polypeptide.

In aspects, the present disclosure also provides for nucleic acids (e.g., DNA, RNA, vectors, viruses, or hybrids thereof, all of which may be isolated, synthetic, or recombinant) that encode in whole or in part one or more modified interferon-α2 polypeptides of the present disclosure and/or chimeric or fusion polypeptide compositions of the present disclosure. In aspects, the nucleic acid further comprises, or is contained within, an expression cassette, a plasmid, and expression vector, or recombinant virus, wherein optionally the nucleic acid, or the expression cassette, plasmid, expression vector, or recombinant virus is contained within a cell, optionally a human cell or a non-human cell, and optionally the cell is transformed with the nucleic acid, or the expression cassette, plasmid, expression vector, or recombinant virus. In aspects, cells are transduced, transfected, or otherwise engineered to contain within one or more of e.g., polypeptides (modified interferon-α2 polypeptides) of the present disclosure; isolated, synthetic, or recombinant nucleic acids, expression cassettes, plasmids, expression vectors, or recombinant viruses as disclosed herein; and/or isolated, synthetic, or recombinant chimeric or fusion polypeptide compositions as disclosed herein. In aspects, the cell can be a mammalian cell, bacterial cell, insect cell, or yeast cell. In aspects, the nucleic acid molecules of the present disclosure can be inserted into vectors and used, for example, as expression vectors or gene therapy vectors. Gene therapy vectors can be delivered to a subject by, e.g., intravenous injection, local administration (U.S. Pat. No. 5,328,470) or by stereotactic injection (Chen S H et al., (1994), Proc Natl Acad Sci USA, 91(8):3054-7, which are herein incorporated by reference in their entirety). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system. Such pharmaceutical compositions can be included in a container, pack, or dispenser together with instructions for administration. In aspects, the present disclosure is directed to a cell comprising a vector of the present disclosure. In aspects, the cell can be a mammalian cell, bacterial cell, insect cell, or yeast cell.

For polynucleotides, a “variant” comprises a deletion and/or addition of one or more nucleotides at one or more internal sites within the polynucleotide sequences of the instant disclosure and/or a substitution of one or more nucleotides at one or more sites in the polynucleotide sequences of the instant disclosure. One of skill in the art will recognize that variants of the polynucleotides of the invention will be constructed such that the open reading frame is maintained. For polynucleotides, conservative variants include those sequences that, because of the degeneracy of the genetic code, encode the amino acid sequence of one of the polypeptides of the invention. Naturally occurring allelic variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis but which still encode a polynucleotide having the desired activity of the invention (i.e., encoding a polypeptide that possesses the desired biological activity, that is, antipathogenic activity, antifungal activity, antialgal activity, and/or enzymatic activity against chitin and/or polyglucuronic acid as described herein). Generally, variants of a particular polynucleotide of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Variants of a particular polynucleotide of the present disclosure (i.e., the reference polynucleotide) can also be evaluated by comparison of the percent sequence identity between the polypeptide encoded by a variant polynucleotide and the polypeptide encoded by the reference polynucleotide. Percent sequence identity between any two polypeptides can be calculated using sequence alignment programs and parameters described elsewhere herein. Where any given pair of polynucleotides of the invention is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

The polynucleotides provided herein (whether RNA, DNA, expression cassettes, vectors, viruses or hybrids thereof) that encode in whole or in part one or more polypeptides of the present disclosure can be isolated from a variety of sources, genetically engineered, amplified, synthetically produced, and/or expressed/generated recombinantly. Recombinant polypeptides generated from these nucleic acids can be individually isolated or cloned and tested for a desired activity. Any recombinant expression system can be used, including e.g. in vitro, bacterial, fungal, mammalian, yeast, insect or plant cell expression systems. In aspects polynucleotides provided herein are synthesized in vitro by well-known chemical synthesis techniques (as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066, all of which are herein incorporated by reference in their entirety). Further, techniques for the manipulation of polynucleotides provided herein, such as, e.g., subcloning, labeling probes (e.g., random-primer labeling using Klenow polymerase, nick translation, amplification), sequencing, hybridization and the like are well described in the scientific and patent literature (see, e.g., Sambrook, ed., Molecular Cloning: A Laboratory Manual (2ND ED.), Vols. 1-3, Cold Spring Harbor Laboratory, (1989); Current Protocols In Molecular Biology, Ausubel, ed. John Wiley & Sons, Inc., New York (1997); Laboratory Techniques In Biochemistry And Molecular Biology: Hybridization With Nucleic Acid Probes, Part I. Theory and Nucleic Acid Preparation, Tijssen, ed. Elsevier, N.Y. (1993), all of which are herein incorporated by reference in their entirety).

In aspects, the present disclosure is directed to a characterized cell line comprising the nucleic acid that encodes for a modified interferon-α2 as disclosed herein. In aspects, said cell line is suitable for the production of a modified interferon-α2 as disclosed herein. In preferred embodiments, a cell line suitable for the production of a modified interferon-α2 as disclosed herein is selected from the set of CHO-K1, HEK293, NS0, BHK, Sp2/0, CAP, and CAP/T. In aspects, the present disclosure is also directed to a method for obtaining a eukaryotic cell line, for producing a modified interferon-α2 as disclosed herein by transformation or transfection of a cell line containing said gene encoding a modified interferon-α2 as disclosed herein, inserted in a suitable expression vector. Preferably, the eukaryotic cell line is a CHO.K1 cell line. In aspects, the present disclosure is directed to a method for producing a modified interferon-α2 as disclosed herein, said method comprising the steps of a) culturing said transformed or transfected eukaryotic cell line with an expression vector containing the gene encoding a modified interferon-α2 polypeptide as disclosed herein, and b) isolating the expressed and secreted modified interferon-α2 polypeptide from the culture medium.

In aspects, the present disclosure is directed a method for purifying a modified interferon-α2 polypeptide as disclosed herein. In aspects, said process of purification of a modified interferon-α2 polypeptide involves purification by immunoaffinity chromatography. In aspects, a process of purification of a modified interferon-α2 polypeptide involves purification by 4-0 immunoaffinity chromatography, wherein the purification by immunoaffinity chromatography comprises the use of anti-nonglycosylated rhIFN-α2b mAb CA5E6 antibody. In aspects, a process of purification of a modified interferon-α2 polypeptide involves purification by immunoaffinity chromatography, wherein the purification by immunoaffinity chromatography comprises the use of anti-hGM-CSF monoclonal antibody (called, mAb CC1H7). In aspects, a process of purification of a modified interferon-α2 polypeptide further comprises the step wherein, following purification (e.g., by immunoaffinity chromatography), the concentration of the purified modified interferon-α2 polypeptide is determined. In preferred embodiments, said determination of the concentration of the purified modified interferon-α2 polypeptide is determined by spectrophotometric quantification.

In aspects, modified interferon-α2 compounds or compositions of the present disclosure (including one or more modified interferon-α2 polypeptides, polynucleotides, microorganism that expresses one or more polypeptides or polynucleotides, expression cassettes, plasmids, expression vectors, chimeric or fusion polypeptides, recombinant viruses and/or pharmaceutical compositions of the present disclosure) can be purified to homogeneity or partially purified. It is understood, however, that preparations in which the modified interferon-α2 compositions are not purified to homogeneity are useful. The critical feature is that the preparation allows for the desired function of the modified interferon-α2, even in the presence of considerable amounts of other components. Thus, the present disclosure encompasses various degrees of purity. In one embodiment, the language “substantially free of cellular material” includes preparations of the modified interferon-α2 having less than about 30% (by dry weight) other proteins (e.g., contaminating protein), less than about 20% other proteins, less than about 10% other proteins, less than about 5% other proteins, less than about 4% other proteins, less than about 3% other proteins, less than about 2% other proteins, less than about 1% other proteins, or any value or range therebetween.

In aspects, a modified interferon-α2 compound or composition of the present disclosure is recombinantly produced, wherein said modified interferon-α2 composition can also be substantially free of culture medium, for example, culture medium represents less than about 20%, less than about 10%, or less than about 5% of the volume of the modified interferon-α2 polypeptide, nucleic acid, or chimeric or fusion polypeptide preparation. The language “substantially free of chemical precursors or other chemicals” includes preparations of the polypeptide, nucleic acid, or chimeric or fusion polypeptide in which it is separated from chemical precursors or other chemicals that are involved in the synthesis of the modified interferon-α2. The language “substantially free of chemical precursors or other chemicals” can include, for example, preparations of modified interferon-α2 polypeptide, nucleic acid, or chimeric or fusion polypeptide having less than about 30% (by dry weight) chemical precursors or other chemicals, less than about 20% chemical precursors or other chemicals, less than about 10% chemical precursors or other chemicals, less than about 5% chemical precursors or other chemicals, less than about 4% chemical precursors or other chemicals, less than about 3% chemical precursors or other chemicals, less than about 2% chemical precursors or other chemicals, or less than about 1% chemical precursors or other chemicals.

In aspects, a modified interferon-α2 polypeptide compound or composition of the present disclosure can be produced by standard recombinant DNA or RNA techniques as are known in the art. For example, DNA or RNA fragments coding for the different polypeptide sequences may be ligated together in-frame in accordance with conventional techniques. In another embodiment, the fusion gene can be synthesized by conventional techniques including automated DNA synthesizers. Alternatively, polymerase chain reaction (PCR) amplification of nucleic acid fragments can be carried out using anchor primers which give rise to complementary overhangs between two consecutive nucleic acid fragments which can subsequently be annealed and re-amplified to generate a chimeric nucleic acid sequence (Short Protocols in Molecular Biology: A Compendium of Methods from Current Protocols in Molecular Biology, (2^{ND}, 1992), FM Asubel et al. (eds), Green Publication Associates, New York, N.Y. (Publ), ISBN: 9780471566355, which is herein incorporated by reference in its entirety). Further, one or more polypeptides (e.g., modified interferon-α2 polypeptide) of the present disclosure (e.g., one or more modified interferon-α2 polypeptides of the present disclosure having a sequence comprising, consisting of, or consisting essentially of one or more of SEQ ID NOS: 2, 4, 6, 8, 14, 16, 18, 20, and 27-42) can be inserted into a heterologous polypeptide or inserted into a non-naturally occurring position of a polypeptide through recombinant techniques, synthetic polymerization techniques, mutagenesis, or other standard techniques known in the art. For example, protein engineering by mutagenesis can be performed using site-directed mutagenesis techniques, or other mutagenesis techniques known in the art (see e.g., James A. Brannigan and Anthony J. Wilkinson., 2002, Protein engineering 20 years on. Nature Reviews Molecular Cell Biology 3, 964-970; Turanli-Yildiz B. et al., 2012, Protein Engineering Methods and Applications, intechopen.com, which are herein incorporated by reference in their entirety).

Moreover, many expression vectors are commercially available that already encode a fusion moiety (e.g., a GST protein). A nucleic acid molecule encoding a modified interferon-α2 of the invention can be cloned into such an expression vector such that the fusion moiety is linked in-frame to the at least one modified interferon-α2. Such linking of the fusion moiety may be done, for example, to improve protein purification yields.

### Pharmaceutical Compositions and Formulations

In aspects, one or more modified interferon-α2 polypeptides, chimeric polypeptides, polynucleotides, microorganism that expresses one or more polypeptides or polynucleotides, expression cassettes, plasmids, expression vectors, and/or recombinant viruses of the present disclosure (hereafter referred to as “modified interferon-α2 compounds or compositions of the present disclosure” or the like) may be comprised in a pharmaceutical composition or formulation. In aspects, pharmaceutical compositions or formulations generally comprise a modified interferon-α2 compound or composition of the present disclosure and a pharmaceutically-acceptable carrier and/or excipient. In aspects, said pharmaceutical compositions are suitable for administration. Pharmaceutically-acceptable carriers and/or

excipients are determined in part by the particular composition being administered, as well as by the particular method used to administer the composition. Accordingly, there is a wide variety of suitable formulations of pharmaceutical compositions for administering the instantly disclosed modified interferon-α2 compositions (see, e.g., Remington's Pharmaceutical Sciences, (18^{TH} Ed, 1990), Mack Publishing Co., Easton, Pa. Publ)). In aspects, the pharmaceutical compositions are generally formulated as sterile, substantially isotonic, and in full compliance with all Good Manufacturing Practice (GMP) regulations of the U.S. Food and Drug Administration.

The terms “pharmaceutically-acceptable,” “physiologically-tolerable,” and grammatical variations thereof, as they refer to compositions, carriers, excipients, and reagents, are used interchangeably and represent that the materials are capable of administration to or upon a subject without the production of undesirable physiological effects to a degree that would prohibit administration of the composition. For example, “pharmaceutically-acceptable excipient” means, for example, an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous. A person of ordinary skill in the art would be able to determine the appropriate timing, sequence and dosages of administration for modified interferon-α2 compositions of the present disclosure.

In aspects, preferred examples of such carriers or diluents include, but are not limited to, water, saline, Ringer's solutions, dextrose solution, and 5% human serum albumin. Liposomes and non-aqueous vehicles such as fixed oils can also be used. The use of such media and compounds for pharmaceutically active substances is well known in the art. Except insofar as any conventional media or compound is incompatible with the modified interferon-α2 compounds or compositions of the present disclosure and as previously described above, use thereof in the compositions is contemplated. Supplementary active compounds can also be incorporated into the compositions.

In aspects, a modified interferon-α2 compound or composition of the present disclosure is formulated to be compatible with its intended route of administration. The modified interferon-α2 compounds or compositions of the present disclosure can be administered by parenteral, topical, intravenous, oral, subcutaneous, intra-arterial, intradermal, transdermal, rectal, intracranial, intrathecal, intraperitoneal, intranasal; vaginally; intramuscular route or as inhalants. In aspects, modified interferon-α2 compounds or compositions of the present disclosure can be injected directly into a particular tissue. In other aspects, intramuscular injection or intravenous infusion may be used for administration of modified interferon-α2 compounds or compositions of the present disclosure. In some methods, modified interferon-α2 compounds or compositions of the present disclosure are administered as a sustained release composition or device, such as but not limited to a Medipad™ device.

In aspects, modified interferon-α2 compounds or compositions of the present disclosure can optionally be administered in combination with other agents that are at least partly effective in treating various medical conditions as described herein. For example, modified interferon-α2 compounds or compositions of the present disclosure can also be administered in conjunction with other agents that stimulate antiviral activity of the immune system, improve pharmacokinetic parameters of the composition, enhance and/or compliment the natural biological activity of interferon-α2, and/or reduce immunogenicity of the composition.

In aspects, solutions or suspensions used for parenteral, intradermal, or subcutaneous application can include, but are not limited to, the following components: a sterile diluent such as water for injection, saline solution, fixed oils, polyethylene glycols, glycerin, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. Examples of excipients can include starch, glucose, lactose, sucrose, gelatin, malt, rice, flour, chalk, silica gel, water, ethanol, DMSO, glycol, propylene, dried skim milk, and the like. The composition can also contain pH buffering reagents, and wetting or emulsifying agents. In aspects, the parenteral preparation can be enclosed in ampoules, disposable syringes or multiple dose vials made of glass or plastic.

In aspects, pharmaceutical compositions or formulations suitable for injectable use include sterile aqueous solutions (where water-soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition is sterile and should be fluid to the extent that easy syringeability exists. It is stable under the conditions of manufacture and storage and is preserved against the contaminating action of microorganisms such as bacteria and fungi. In aspects, modified interferon-α2 formulations may include aggregates, fragments, breakdown products and post-translational modifications, to the extent these impurities have reduced immunogenicity and high relative antiviral activity that is similar to pure modified interferon-α2. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. In many cases, it will be preferable to include isotonic compounds, e.g., sugars, polyalcohols such as mannitol, sorbitol, and sodium chloride, in the composition. Prolonged absorption of the injectable compositions can be brought about by including in the composition a compound that delays absorption, e.g., aluminum monostearate and gelatin.

In aspects, sterile injectable solutions can be prepared by incorporating the modified interferon-α2 compounds or compositions of the present disclosure in the required amount in an appropriate solvent with one or a combination of ingredients enumerated above, as required, followed by filtered sterilization. Generally, dispersions are prepared by incorporating the binding agent into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof. Further, modified interferon-α2 compounds or compositions of the present disclosure can be administered in the form of a depot injection or implant preparation that can be formulated in such a manner as to permit a sustained or pulsatile release of the active ingredient

In aspects, solutions or suspensions of pharmaceutical compositions or formulations are maintained at a pH at which the modified interferon-α2 polypeptide is in its natural structural conformation. In aspects, said pH is maintained below pH 10. In aspects, said pH is maintained below pH 7. In aspects, said pH is maintained between pH 3-10. In aspects, said pH is maintained between pH 4-9. In aspects, said pH is maintained between pH 5-8. In aspects, said pH is maintained between pH 6-7.5. In aspects, a buffer is provided to maintain the pH at a desired level. In aspects, said buffer is a phosphate buffer. In aspects, said buffer is an acetate buffer.

In aspects, solutions or suspensions of pharmaceutical compositions or formulations include surface adsorption inhibitors. In aspects, said surface adsorption inhibitors are provided that inhibit the adsorption of components of the pharmaceutical compositions or formulations by surfaces that enclose the compositions or formulations (such as ampoules, syringes, or vials made of glass or plastic). In preferred embodiments, said surface adsorption inhibitors are provided that inhibit the adsorption of one or more modified interferon-α2 polypeptides by glass surfaces that enclose the compositions or formulations. In aspects, the pharmaceutical compositions or formulations are enclosed in ampoules, syringes, or vials made of borosilicate glass, and the pharmaceutical compositions or formulations include a surface adsorption inhibitor (e.g., a surface adsorption inhibitor that inhibits the adsorption of one or more modified interferon-α2 polypeptides by the borosilicate glass surface). In aspects, said surface adsorption inhibitor is Polysorbate 80. In aspects, said surface adsorption inhibitor is albumin.

In aspects, solutions or suspensions of pharmaceutical compositions or formulations include degradation inhibitors. In aspects, degradation inhibitors are provided that inhibit the degradation of a modified interferon-α2 polypeptide. In aspects, degradation inhibitors are provided that inhibit the oxidative degradation of a modified interferon-α2 polypeptide. In aspects, degradation inhibitors are provided that inhibit the oxidative degradation of a modified interferon-α2 polypeptide, wherein said degradation inhibitor is benzyl alcohol.

In aspects, pharmaceutical compositions or formulations include a sterile powder for the extemporaneous preparation of sterile injectable solutions or dispersion, wherein said sterile powder comprises: a dry powder formulation of one or more modified interferon-α2 polypeptides, a bulking agent, and a surface adsorption inhibitor. In aspects, said bulking agent is glycine. In aspects, said surface adsorption inhibitor is albumin. In aspects, said sterile powder further comprises one or more antimicrobial preservatives. In aspects, said one or more antimicrobial preservatives are selected from the group comprised of: m-cresol, benzyl alcohol, and phenol. In aspects, said sterile powder further comprises sodium phosphate dibasic and sodium phosphate monobasic. In aspects, said sterile powder is provided as a tablet-like solid that is whole, in pieces, and/or in a loose powder. In aspects, said dry powder formulation of one or more modified interferon-α2 polypeptides is a lyophilized powder. In aspects, said one or more modified interferon-α2 polypeptides are provided that have a desired specific activity. In aspects, said sterile powder is stored at a cold temperature prior to administration to a subject. In aspects, said sterile powder is stored at a temperature in the range of 2° C.-8° C. prior to administration to a subject. In aspects, prior to administration to a subject, said sterile powder is reconstituted with a diluent to provide a sterile solution. In aspects, said reconstitution is accomplished by dissolving the sterile powder in the diluent (e.g., by stirring, swirling, inverting, shaking, vortexing, or other means known and understood in the art) to produce the sterile solution. In aspects, said diluent comprises one or more components selected from the group comprised of: sterile water, sodium chloride, sodium phosphate dibasic, sodium phosphate monobasic, EDTA, polysorbate 80, and m-cresol. In aspects, said resuspension is performed in a single-use vial, ampoule, or syringe. In aspects, said sterile solution provides one or more modified interferon-α2 polypeptides at a desired concentration. In aspects, said desired concentration of modified interferon-α2 polypeptide is 1-100 million IU/mL. In aspects, said desired concentration of a modified interferon-α2 polypeptide is 10-50 million IU/mL. In aspects, said desired concentration of a modified interferon-α2 polypeptide is 1-10 million IU/mL. In aspects, said desired concentration of a modified interferon-α2 polypeptide is decreased for a maintenance dose during maintenance treatment of a condition in a subject. In aspects, said sterile solution is stored at a cold temperature prior to administration to a subject. In aspects, said sterile solution is stored at a temperature in the range of 2° C.-8° C. prior to administration to a subject.

In aspects, pharmaceutical compositions or formulations include solutions or suspensions comprising one or more modified interferon-α2 polypeptides and one or more components, wherein said components are selected from the group comprised of: sterile water, sodium chloride, sodium phosphate dibasic, sodium phosphate monobasic, EDTA, one or more surface adsorption inhibitors (e.g., polysorbate 80), one or more antimicrobial preservatives (e.g., m-cresol), one or more bulking agents, and one or more degradation inhibitors. In aspects, said solution or suspension comprises: one or more modified interferon-α2 polypeptides, sterile water, sodium chloride, sodium phosphate dibasic, sodium phosphate monobasic, EDTA, polysorbate 80, and m-cresol. In aspects, said one or more modified interferon-α2 polypeptides are provided that have a desired specific activity. In aspects, said solution or suspension provides said one or more modified interferon-α2 polypeptides at a desired concentration. In aspects, said desired concentration of a modified interferon-α2 polypeptide is 1-100 million IU/mL. In aspects, said desired concentration of a modified interferon-α2 polypeptide is 10-50 million IU/mL. In aspects, said desired concentration of a modified interferon-α2 polypeptide is 1-10 million IU/mL. In aspects, said desired concentration of a modified interferon-α2 polypeptide is decreased for a maintenance dose during maintenance treatment of a condition in a subject. In aspects, said solution or suspension is stored at a cold temperature prior to administration to a subject. In aspects, said solution or suspension is stored at a temperature in the range of 2° C.-8° C. prior to administration to a subject.

In aspects, solutions or suspensions of pharmaceutical compositions or formulations comprise: one or more modified interferon-α2 polypeptides, a salt, and a buffer. In aspects, said buffer is provided to maintain the pH at a desired level. In aspects, said buffer is phosphate buffer and said salt is sodium chloride.

In aspects, pharmaceutical compositions or formulations include a sterile powder for the extemporaneous preparation of sterile injectable solutions or dispersion, wherein said sterile powder comprises a dry powder formulation of one or more modified interferon-α2 polypeptides. In aspects, said sterile powder further comprises one or more components selected from the group comprised of: dibasic sodium phosphate anhydrous, monobasic sodium phosphate dihydrate, sucrose, and polysorbate 80. In aspects, said sterile powder is provided as a tablet-like solid that is whole, in pieces, and/or in a loose powder. In aspects, said dry powder formulation of one or more modified interferon-α2 polypeptides is a lyophilized powder. In aspects, said one or more modified interferon-α2 polypeptides are provided that have a desired specific activity. In aspects, said sterile powders are stored at a cold temperature prior to administration to a subject. In aspects, said sterile powders stored at a temperature in the range of 2° C.-8° C. prior to administration to a subject. In aspects, said sterile powders are stored at room temperature prior to resuspension. In aspects, said sterile powders stored at a temperature in the range of 15° C.-30° C. prior to resuspension. In aspects, prior to administration to a subject, said sterile powder is reconstituted with a diluent to provide a sterile solution. In aspects, said reconstitution is accomplished by dissolving the sterile powder in the diluent (e.g., by stirring, swirling, inverting, shaking, vortexing, or other means known and understood in the art) to produce the sterile solution. In aspects, said diluent comprises sterile water. In aspects, said resuspension is performed in a single-use vial, ampoule, or syringe. In aspects, said resuspension is performed in a dual-chamber cartridge, wherein a first chamber contains said sterile powder and a second chamber contains said diluent, and wherein, prior to injection, the components of the two chambers are combined to produce a sterile solution. In aspects, said dual chamber cartridge is used to inject said sterile solution into a subject via an injection apparatus that is a part of the dual-chamber cartridge. In aspects, said sterile solution provides said one or more modified interferon-α2 polypeptides at a desired concentration. In aspects, said desired concentration of a modified interferon-α2 polypeptide is 50-500 mcg/mL. In aspects, said desired concentration of a modified interferon-α2 polypeptide is 100-300 mcg/mL. In aspects, said desired concentration of a modified interferon-α2 polypeptide is 100-2000 mcg/mL. In aspects, said desired concentration of a modified interferon-α2 polypeptide is 400-1200 mcg/mL. In aspects, said sterile solution is stored at a cold temperature prior to administration to a subject. In aspects, said sterile solution is stored at a temperature in the range of 2° C.-8° C. prior to administration to a subject.

In aspects, pharmaceutical compositions or formulations of a modified interferon-α2 compound or composition of the present disclosure are co-administered with one or more other pharmaceutical compositions of formulations. In aspects, said one or more other pharmaceutical compositions or formulations are selected from the group consisting of: ribavirin (e.g., REBETOL®), Pegintron®, and INTRON-A®.

In aspects, oral compositions generally include an inert diluent or an edible carrier and can be enclosed in gelatin capsules or compressed into tablets. In aspects, for the purpose of oral therapeutic administration, the binding agent can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding compounds, and/or adjuvant materials can be included as part of the composition. In aspects, the tablets, pills, capsules, troches and the like can contain any of the following ingredients, or compounds of a similar nature: a binder such as microcrystalline cellulose, gum tragacanth or gelatin; an excipient such as starch or lactose, a disintegrating compound such as alginic acid, Primogel or corn starch; a lubricant such as magnesium stearate or Sterotes; a glidant such as colloidal silicon dioxide; a sweetening compound such as sucrose or saccharin; or a flavoring compound such as peppermint, methyl salicylate or orange flavoring.

For administration by inhalation, modified interferon-α2 compounds or compositions of the present disclosure can be delivered in the form of an aerosol spray from pressured container or dispenser that contains a suitable propellant, e.g., a gas such as carbon dioxide, or a nebulizer.

In aspects, systemic administration of modified interferon-α2 compounds or compositions of the present disclosure can also be by transmucosal or transdermal means. For transmucosal or transdermal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of nasal sprays or suppositories. For transdermal administration, the modified interferon-α2 compounds or compositions of the present disclosure may be formulated into ointments, salves, gels, or creams and applied either topically or through transdermal patch technology as generally known in the art.

In aspects, modified interferon-α2 compounds or compositions of the present disclosure can also be prepared in the form of suppositories (e.g., with conventional suppository bases such as cocoa butter and other glycerides) or retention enemas for rectal delivery.

In aspects, modified interferon-α2 compounds or compositions of the present disclosure are prepared with carriers that protect the modified interferon-α2 compositions against rapid elimination from the body, such as a controlled-release formulation, including implants and microencapsulated delivery systems. Biodegradable, biocompatible polymers can be used, such as, for example, ethylene vinyl acetate, polyanhydrides, polyglycolic acid, collagen, polyorthoesters, and polylactic acid. Methods for preparation of such formulations will be apparent to those skilled in the art. The materials can also be obtained commercially, e.g., from Alza Corporation and Nova Pharmaceuticals, Inc. Liposomal suspensions (including liposomes targeted to infected cells with monoclonal antibodies to viral antigens) can also be used as pharmaceutically-acceptable carriers. These can be prepared according to methods known to those skilled in the art (U.S. Pat. No. 4,522,811, which is herein incorporated by reference in its entirety). In aspects, the modified interferon-α2 compounds or compositions of the present disclosure can be implanted within or linked to a biopolymer solid support that allows for the slow release of the modified interferon-α2 compositions to the desired site.

In aspects, it is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of binding agent calculated to produce the desired therapeutic effect in association with the required pharmaceutical carrier. The specification for the dosage unit forms of the instant disclosure are dictated by and directly dependent on the unique characteristics of the binding agent and the particular therapeutic effect to be achieved, and the limitations inherent in the art of compounding such modified interferon-α2 compounds or compositions of the present disclosure for the treatment of a subject.

### METHODS OF USE

The modified interferon-α2 compounds or compositions of the present disclosure (including one or more modified interferon-α2 polypeptides, polynucleotides, microorganism that expresses one or more polypeptides or polynucleotides, expression cassettes, plasmids, expression vectors, chimeric or fusion polypeptides, recombinant viruses and/or pharmaceutical compositions of the present disclosure) find use in protecting/treating against melanomas, melanomas (including malignant melanoma), acute and chronic hepatitis C (including in patients with compensated liver disease), acute and chronic hepatitis B, acute and chronic non-A, non-B hepatitis, Kaposi's sarcoma (including AIDS-related Kaposi's sarcoma), multiple sclerosis, genital warts, leukemia (including Hairy cell leukemia), lymphomas (including follicular lymphoma), condylomata acumiate, and other viral infections (including SARS-CoV-2 infection ZIKV infection, CHIKV infection, or influenza A infection). In aspects, the present disclosure provides the use of a modified interferon-α2 compounds or compositions of the present disclosure, such as disclosed herein, for manufacturing a medicament for the treatment of against melanomas, melanomas (including malignant melanoma), acute and chronic hepatitis C (including in patients with compensated liver disease), acute and chronic hepatitis B, acute and chronic non-A, non-B hepatitis, Kaposi's sarcoma (including AIDS-related Kaposi's sarcoma), multiple sclerosis, genital warts, leukemia (including Hairy cell leukemia), lymphomas (including follicular lymphoma), condylomata acumiate, and other viral infections (including SARS-CoV-2 infection ZIKV infection, CHIKV infection, or influenza A infection).

In aspects, the present disclosure is directed to methods of preventing or treating one or more medical conditions in a subject comprising administering one or more modified interferon-α2 compounds or compositions of the present disclosure, and preventing or treating the medical condition in a subject by said step of administering said one or more modified interferon-α2 compounds or compositions of the present disclosure. The medical condition can be, for example against melanomas, melanomas (including malignant melanoma), acute and chronic hepatitis C (including in patients with compensated liver disease), acute and chronic hepatitis B, acute and chronic non-A, non-B hepatitis, Kaposi's sarcoma (including AIDS-related Kaposi's sarcoma), multiple sclerosis, genital warts, leukemia (including Hairy cell leukemia), lymphomas (including follicular lymphoma), condylomata acumiate, and other viral infections (including SARS-CoV-2 infection ZIKV infection, CHIKV infection, or influenza A infection). In aspects, the modified interferon-α2 compounds or compositions of the present disclosure can be used with in conjunction with other proteins or compounds used for treating a subject with the medical condition in order to reduce adverse events or enhance the efficacy of the co-administered compound.

In a particular aspect, the present disclosure is directed to, for example, methods of treating chronic hepatitis C, said method comprising administering one or more modified interferon-α2 compounds or compositions of the present disclosure, and preventing or treating chronic hepatitis C in a subject by said step of administering said one or more modified interferon-α2 compounds or compositions of the present disclosure. The modified interferon-α2 compounds or compositions of the present disclosure can be used with in conjunction with other proteins or compounds used for treating a subject with chronic hepatitis C in order to reduce adverse events or enhance the efficacy of the co-administered compound. In aspects, the modified interferon-α2 compounds or compositions of the present disclosure (e.g., GMOP-IFN-alpha-2 variants, IFN-alpha-2 variants, etc.) lack antiproliferative properties while preserving antiviral activity, representing interesting therapeutic alternatives for chronic Hepatitis C treatment. In aspects, the modified interferon-α2 compounds or compositions of the present disclosure demonstrate high relative antiviral activity with reduced immunogenicity in chronic Hepatitis C treatment.

In aspects, the present disclosure is directed to, for example, methods of treating chronic hepatitis B, said method comprising administering one or more modified interferon-α2 compounds or compositions of the present disclosure, and preventing or treating chronic hepatitis B in a subject by said step of administering said one or more modified interferon-α2 compounds or compositions of the present disclosure. The modified interferon-α2 compounds or compositions of the present disclosure can be used with in conjunction with other proteins or compounds used for treating a subject with chronic hepatitis B in order to reduce adverse events or enhance the efficacy of the co-administered compound. In aspects, a modified interferon-α2 composition of the present disclosure (e.g., GMOP-IFN-alpha-2 variants, IFN-alpha-2 variants) lack antiproliferative properties while preserving antiviral activity, representing interesting therapeutic alternatives for chronic Hepatitis B treatment. In aspects, the modified interferon-α2 compounds or compositions of the present disclosure demonstrate high relative antiviral activity with reduced immunogenicity in chronic Hepatitis B treatment.

Emerging viral infections with agents such as SARS-CoV-2, ZIKV, CHIKV and influenza A among others, represent a relevant world-wide public health concern. This is due to the rapid spread of their etiologic agents to new areas, the increasing number of human infections and the lack of new therapeutic treatments and/or effective vaccines. In aspects, the present disclosure is directed to, for example, methods of treating SARS-CoV-2 infection (and/or related diseases caused by SARS-CoV-2, including COVID-19), ZIKV, CHIKV or influenza A, said method comprising administering one or more modified interferon-α2 compounds or compositions of the present disclosure, and preventing or treating said infection or disease in a subject by said step of administering said one or more modified interferon-α2 compounds or compositions of the present disclosure. In aspects, the modified interferon-α2 compounds or compositions of the present disclosure can be used with in conjunction with other proteins or compounds used for treating a subject with a medical condition in order to reduce adverse events or enhance the efficacy of the co-administered compound. In aspects, modified interferon-α2 compounds or compositions of the present disclosure (e.g., GMOP-IFN-alpha-2 variants, IFN-alpha-2 variants) lack antiproliferative properties while preserving antiviral activity, representing interesting therapeutic alternatives for SARS-CoV-2 (and/or related diseases caused by SARS-CoV-2, including COVID-19), ZIKV, CHIKV, or influenza A treatment. In aspects, the one or more compounds or compositions of the present disclosure as previously described demonstrate high relative antiviral activity with reduced immunogenicity in ZIKV, CHIKV or influenza A treatment.

In aspects of the above-described methods, said modified interferon-α2 compounds or compositions of the present disclosure are co-administered with one or more other pharmaceutical compositions of formulations. In aspects, said one or more other pharmaceutical compositions or formulations are selected from the group consisting of: ribavirin (e.g., REBETOL®), PegIntron®, and INTRON-A®.

The methods described herein can be performed, e.g., by utilizing pre-packaged kits comprising at least one pharmaceutical formulation or composition for treatment and/or prevention of a disease as described herein (including a melanoma or viral infection and/or related diseases), which can be conveniently used, e.g., in clinical settings to treat subjects exhibiting symptoms or family history of a medical condition described herein. In one embodiment, the kit further comprises instructions for use of the at least one modified interferon-α2 composition of the instant disclosure to treat subjects exhibiting symptoms or family history of a medical condition described herein.

### EXEMPLIFICATION

The examples that follow are not to be construed as limiting the scope of the invention in any manner. In light of the present disclosure, numerous embodiments within the scope of the claims will be apparent to those of ordinary skill in the art.

### (1) In-Silico Identification of Immunogenic Regions of Therapeutics

T cells specifically recognize epitopes presented by antigen presenting cells (APCs) in the context of MHC (Major Histocompatibility Complex) Class II molecules. These T-helper epitopes can be represented as linear sequences comprising 7 to 30 contiguous amino acids that fit into the MHC Class II binding groove. A number of computer algorithms have been developed and used for detecting Class II epitopes within protein molecules of various origins (De Groot A S et al., (1997), AIDS Res Hum Retroviruses, 13(7):539-41; Schafer J R et al., (1998), Vaccine, 16(19):1880-4; De Groot A S et al., (2001), Vaccine, 19(31):4385-95; De Groot A S et al., (2003), Vaccine, 21(27-30):4488-504). These “in silico” predictions of T-helper epitopes have been successfully applied to the design of vaccines and the de-immunization of therapeutic proteins, i.e. antibody-based drugs, Fc fusion proteins, anticoagulants, blood factors, bone morphogenetic proteins, engineered protein scaffolds, enzymes, growth factors, hormones, interferons, interleukins, and thrombolytics (Dimitrov D S, (2012), Methods Mol Biol, 899:1-26).

The EpiMatrix™ system (EpiVax, Providence, R.I.) is a set of predictive algorithms encoded into computer programs useful for predicting class I and class II HLA ligands and T cell epitopes. The EpiMatrix™ system uses 20×9 coefficient matrices in order to model the interaction between specific amino acids (20) and binding positions within the HLA molecule (9). In order to identify putative T cell epitopes resident within any given input protein, the EpiMatrix™ System first parses the input protein into a set of overlapping 9-mer frames where each frame overlaps the last by eight amino acids. Each frame is then scored for predicted affinity to one or more common alleles of the human HLA molecule; typically DRB1′0101, DRB1*0301, DRB1*0401, DRB1*0701, DRB1*0801, DRB1*1101, DRB11301, and DRB1*1501 (Mack et al., (2013), Tiss Antig, 81(4):194-203). Briefly, for any given 9-mer peptide specific amino acid codes (one for each of 20 naturally occurring amino acids) and relative binding positions (1-9) are used to select coefficients from the predictive matrix. Individual coefficients are derived using a proprietary method similar to, but not identical to, the pocket profile method first developed by Stumiolo (Stumiolo T et al., 1999, Nat Biotechnol, 17(6):555-61). Individual coefficients are then summed to produce a raw score. EpiMatrix™ raw scores are then normalized with respect to a score distribution derived from a very large set of randomly generated peptide sequences. The resulting “Z” scores are normally distributed and directly comparable across alleles. EpiMatrix™ peptide scoring. It was determined that any peptide scoring above 1.64 on the EpiMatrix™ “Z” scale (approximately the top 5% of any given peptide set) has a significant chance of binding to the MHC molecule for which it was predicted and are designated a “hit.” Peptides scoring above 2.32 on the scale (the top 1%) are extremely likely to bind; most published T cell epitopes fall within this range of scores. Previous studies have also demonstrated that EpiMatrix™ accurately predicts published MHC ligands and T cell epitopes.

Identification of T cell Epitope Clusters. Potential T cell epitopes are not randomly distributed throughout protein sequences but instead tend to “cluster.” T cell epitope “clusters” range from 9 to roughly 30 amino acids in length and, considering their affinity to multiple alleles and across multiple frames, contain anywhere from 4 to 40 binding motifs. Following epitope mapping, the result set produced by the EpiMatrix™ algorithm was screened for the presence of T cell epitope clusters and EpiBars™ by using a proprietary algorithm known as Clustimer™. Briefly, the EpiMatrix™ scores of each 9-mer peptide analyzed are aggregated and checked against a statistically derived threshold value. High scoring 9mers are then extended one amino acid at a time. The scores of the extended sequences are then re-aggregated and compared to a revised threshold value. The process is repeated until the proposed extension no longer improves the overall score of the cluster. Regions of high immunogenic potential, defined as having a score above 10 (including multiple ‘hits’ against many different HLA DR alleles), were identified as T cell epitope clusters. They contain significant numbers of putative T cell epitopes and EpiBars™ indicating a high potential for MHC binding and T cell reactivity.

Prediction of Amino Acids Implicated in HLA Binding. The contribution of each amino acid in these regions to HLA binding was evaluated using OptiMatrix tool (part of the EpiVax ISPRI toolkit for deimmunization). OptiMatrix begins with looking at “critical” residues, which contribute most to MHC binding affinity across multiple 9-mer frames and multiple HLA alleles. The program then iteratively substitutes all 19 alternative amino acids in any given position of a protein sequence (with operator-defined input that may limit the list to naturally conserved variants) and then re-analyzes the predicted immunogenicity of the sequence, following that change. To avoid a negative effect on protein structure and consequently in biological activity a comprehensive search in literature for critical residues was also conducted, which identified amino acids that were not candidates for modification.

### Example 1. In Silico Immunogenicity Prediction and Deimmunized Proteins Design

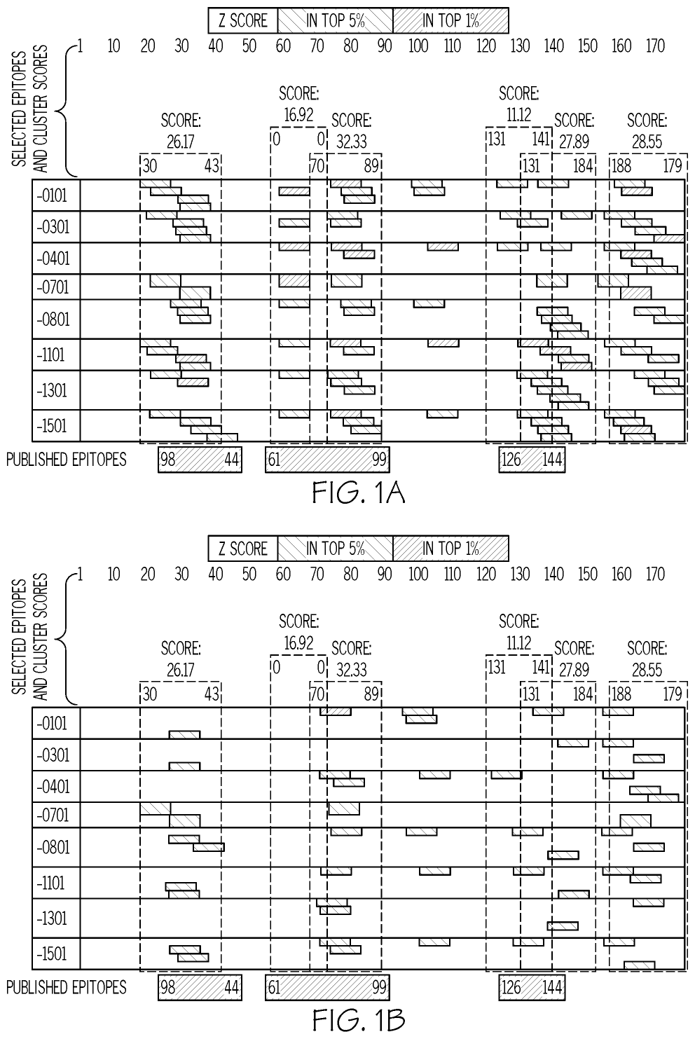

Peptide binding to HLA molecules is the critical first step required for a T cell response. In fact, one of the most critical determinants of protein immunogenicity is the strength of peptide binding to MHC molecules (Lazarski C A et al, (2005) Immunity. 23: 29-40). In order to analyze the potential immunogenicity of GMOP-IFN (SEQ ID NO: 10), the complete amino acid sequence was screened using EpiMatrix. This study revealed a high content of T cell epitopes in the protein sequence (FIG. 1A). A further analysis using the ClustiMer algorithms allowed for the identification of putative 9-mer MHC binding peptides and their combination into cluster regions. A total of six clusters were defined, spanning the following residues of GMOP-IFN (SEQ ID NO: 10): 20-43, 58-72, 70-89, 121-141, 131-154, 158-179. Five out of six predicted MHC binding clusters overlapped with previously reported T cell epitopes.

Then, using OptiMatrix, critical residues were identified that disrupted or reduced MHC II binding affinity. Among the changes suggested by OptiMatrix, modifications that were not identified as critical for biological activity or receptor binding were selected. These results were considered along with the ClustiMer MHC binding cluster predictions. Based on this comparison, ten cites for modification in GMOP-IFN-α2b (SEQ ID NO: 10) were selected, which correspond to the following positions in the amino acid sequence: 23, 31, 61, 79, 80, 131, 142, 137, 161 and 171. These ten mutations were introduced into the GMOP-IFN-α2b sequence (SEQ ID NO: 10) in different combinations to produce the GMOP-IFN-2b variants. Modifications were made that mutated each amino acid to alanine (except for the modification at position 161, in which the amino acid was mutated to threonine). All these mutations were introduced to generate GMOP-IFN-VAR1 (SEQ ID NO: 2) and the impact of the mutations on T cell epitope content is illustrated in (FIG. 1B).

It was discovered that the following modifications in the hIFN-α2b molecule (SEQ ID NO: 12) were critical for binding to specific HLA molecules: L9A, F47A, L117A, F123A and L128A. As such, these modifications (corresponding to amino acids at positions L23A, F61A, L131A, F137A, and L142A in GMOP-IFN-α2b of SEQ ID NO: 10) were mutated to develop GMOP-IFN-VAR2 (SEQ ID NO: 4). Two additional protein variants were also produced, GMOP-IFN-VAR3 (SEQ ID NO: 6) and GMOP-IFN-VAR4 (SEQ ID NO 8), both carrying seven mutations, in order to reduce the antigenicity of clusters 158-179 and 70-89, respectively. The modifications to produce GMOP-IFN-VAR3 (SEQ ID NO: 6) were: L23A, F61A, L131A, F137A, L142A, I161T, and L171A. The modifications to produce GMOP-IFN-VAR4 (SEQ ID NO: 8) were: L23A, F61A, N79A, L80A, L131A, F137A, and L142A. Table 1 summarizes GMOP-IFN-α2b variants created.

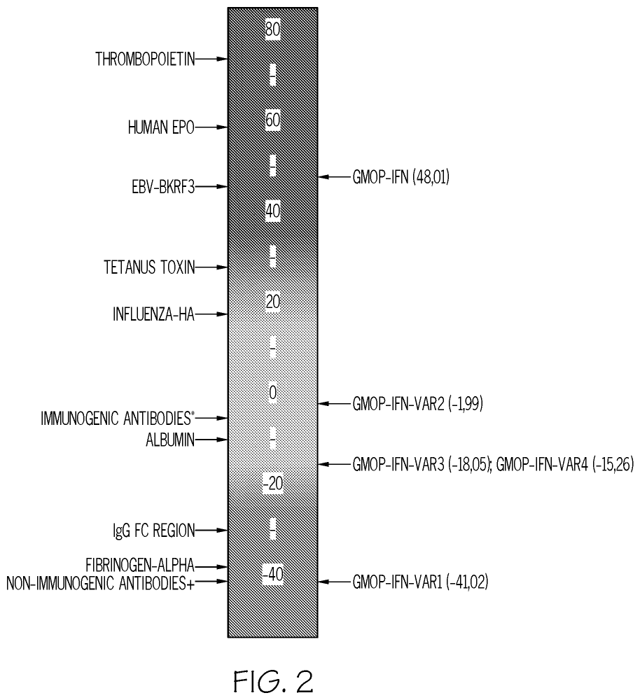

Immunogenicity scores for each of the variants was calculated using EpiMatrix, as described above. As shown in FIG. 2, the EpiMatrix immunogenicity global score for each variant is markedly reduced in comparison with the original molecule.

TABLE 1
GMOP-IFN-α2b Variants

| | | GMOP-IFN-α2b | |
|---|---|---|---|
| Variant | SEQ | Modification | Amino Acid Sequence |
| Name | ID NO | Cites | (GMOP in italic; mutations in bold) |
| GMOP- | 2 | 23, 31, 61, | APARSPSPSTQPWECDLPQTHSAGSRRTLMALAQMRRISLFSCLKD |
| IFN- | | 79, 80, 131, | RHDFGFPQEEFGNQAQKAETIPVLHEMIQQIFAAFSTKDSSAAWDE |
| VAR1 | | 137, 142, | TLLDKFYTELYQQLNDLEACVIQGVGVTETPLMKEDSIAAVRKYAQ |
| | | 161, 171 | RITAYLKEKKYSPCAWEVVRAETMRSFSLSTNAQESLRSKE |
| GMOP- | 4 | 23, 61, 131, | APARSPSPSTQPWECDLPQTHSAGSRRTLMLLAQMRRISLFSCLKD |
| IFN- | | 137, 142 | RHDFGFPQEEFGNQAQKAETIPVLHEMIQQIFNLFSTKDSSAAWDE |
| VAR2 | | | TLLDKFYTELYQQLNDLEACVIQGVGVTETPLMKEDSIAAVRKYAQ |
| | | | RITAYLKEKKYSPCAWEVVRAEIMRSFSLSTNLQESLRSKE |
| GMOP- | 6 | 23, 61, 131, | APARSPSPSTQPWECDLPQTHSAGSRRTLMLLAQMRRISLFSCLKD |
| IFN- | | 137, 142,161, | RHDFGFPQEEFGNQAQKAETIPVLHEMIQQIFNLFSTKDSSAAWDE |
| VAR3 | | 171 | TLLDKFYTELYQQLNDLEACVIQGVGVTETPLMKEDSIAAVRKYAQ |
| | | | RITAYLKEKKYSPCAWEVVRAETMRSFSLSTNAQESLRSKE |
| GMOP- | 8 | 23, 61, 79, | APARSPSPSTQPWECDLPQTHSAGSRRTLMLLAQMRRISLFSCLKD |
| IFN- | | 80, 131, 137, | RHDFGFPQEEFGNQAQKAETIPVLHEMIQQIFAAFSTKDSSAAWDE |
| VAR4 | | 142 | TLLDKFYTELYQQLNDLEACVIQGVGVTETPLMKEDSIAAVRKYAQ |
| | | | RITAYLKEKKYSPCAWEVVRAEIMRSFSLSTNLQESLRSKE |

It should be clarified that the modifications/substitutions presented by the deimmunized variants of GMOP-IFN-2b correspond to those amino acids not involved in the biological structure or function of cytokine, as previously described in detail. That is, this experiment and these mutations can be performed on any of the modified IFN-α2 polypeptides (or related modified IFN-α2 compounds and compositions) as disclosed herein, for example including the following variants: IFN-α2b, GMOP-IFN-α2b, or any other variant of IFN-α2 (including IFN-α2a, GMOP-IFN-α2a, IFN-α2c, and GMOP-IFN-α2c) with or without one or more GMOP sequences attached.

### (2) Gene Expression, and Protein Production, Purification, and Characterization.

Cell Culture. Cell culture Chinese hamster ovary (CHO-K1) cells were grown in basal culture medium previously described (Kratje R B, Wagner R, (1992), Biotechnol. Bioeng. 39: 233-242) supplemented with 5% (v/v) fetal calf serum (FCS) (PAA, Argentina). Human embryonic kidney (HEK293T) cells were cultured in DMEM supplemented with 10% (v/v) FCS and 2 mM glutamine. Madine Darby bovine kidney (MDBK) cells were grown in minimum essential medium (MEM; Gibco, USA) supplemented with 10% (v/v) FCS. Bioassays were performed using MEM supplemented with 2% (v/v) FCS (assay medium). The human Daudi cell line was maintained in RPMI 1640 medium (Gibco) plus 10% (v/v) FCS. All cells were incubated at 37° C. in humidified 5% CO2.

Construction of lentiviral vectors and assembly of lentiviral particles. Plasmids carrying the hl FN-α2b encoding sequence (GeneWiz, USA) were digested with SalI and XbaI enzymes and the released DNA fragments corresponding to each GMOP-IFN variant were cloned into a lentiviral plasmid (pLV) (A: Oberbek A. (2011) BiotechnolBioeng 108(3):600-610., B: Chusainow J. (2009) BiotechnolBioeng 102(4):1182-1196). All construct identities were verified by DNA sequencing. Research grade HIV-based LV particles containing the three hIFN-α2b analogs transgenes were produced following the protocol suggested by Naldini et al. (1996, Science, 272: 263-267) and Dull et al. (1998, J. Virol. 72: 8463-71). Adherent HEK293T cells were cultured in 10 cm-plates and simultaneously co-transfected with four plasmids: the packaging plasmid (pMDLg/pRRE) (Dull et al. (1998), J. Virol. 72: 8463-71), the Rev-expressing plasmid (pRSV-Rev) (Naldini et al. (1996), Science, 272: 263-267), the envelop plasmid expressing VSV-G (pMD2.G) (Dull et al. (1998), J. Virol. 72: 8463-71), and the corresponding transfer vectors containing the transgenes (pLVs). All plasmids were introduced into the cells by liposome-mediated gene transfer, using LipofectAMINE 2000 Reagent (Invitrogen, USA), according to the supplier's instructions. Supernatants containing lentiviral particles (LVPs) were harvested 72 h post-transfection.
Lentiviral transduction. Transductions were carried out by incubating 6.0×10⁴ cells per well seeded onto 6-well plates (Greiner) with 1 ml of supernatants containing LVPs. Twenty-four hours post-transduction, medium were replaced with fresh medium. In order to eliminate the remaining wild type cells, 96 h post-transduction a selective pressure process was started by replacing supernatants with fresh growth medium containing 10 μg·ml-1 puromycin (Sigma Aldrich, USA). Selective medium was changed every 3-4 days with increasing puromycin concentrations until control cell death.
GMOP-IFN variants production and purification. Transduced cells were expanded for GMOP-IFN variants production and the productivity of each cell line was evaluated by determination of rhIFN-α2b concentration and cell counting. Cells were grown until confluence in 500 cm² triple flasks using growth medium. The medium was then changed to basal medium supplemented with 0.5% (v/v) FCS (production medium). Every 48 or 72 h, conditioned medium was harvested and replaced with fresh production medium. Harvests were clarified by centrifugation and stored at −20° C. Protein was purified by immunoaffinity chromatography employing the anti-nonglycosylated rhIFN-α2b mAb CA5E6 (which has proved to bind effectively a wide variety of IFN mutants) coupled to CNBr-activated Sepharose 4B (GE Healthcare) as previously described (Ceaglio N et al., (2008), Biochimie., 90: 437-449). The concentration of purified GMOP-IFN variants was determined by spectrophotometric quantification.
rhIFN-α sandwich ELISA. GMOP-IFN variants yields from culture supernatants were quantified by a specific sandwich ELISA assay as described by Ceaglio et al. (2008, Biochimie. 90: 437-449.) The sandwich ELISA assay is based on the capture of IFN-α2b (in its different versions) by the monoclonal antibody (mAb) CA5E6 immobilized on polystyrene plates and its subsequent recognition by immunoglobulins (Igs) present in a rabbit anti-IFN-2b polyclonal serum (C7).

Flat-bottomed polystyrene plates of 96 wells (Greiner) were sensitized with 1001 of mAb CA5E6 1 g·ml⁻¹ (100 ng/well) diluted in Na₂CO₃/NaHCO_{3 50} mM pH 9.6 solution (sensitization solution). It was incubated for 1 hour at 37° C. and all night at 4° C.

The blocking of non-specific interaction sites was performed with 200 L per well of a bovine serum albumin solution (BSA, Sigma) 1% (P/V) in PBS (blocking solution). It was incubated for 1 hour at 37° C.

The first incubation was performed by adding 100 l of successive dilutions 1:2 of the ifn-2b standard of bacterial origin (Gema Biotech, Argentina) from 10 to ng·ml⁻¹ to 0.078 ng·ml⁻¹, and from the samples to be analyzed. To do this, a 0.1% BSA (P/V) solution was used in PBS with the addition of Tween 20 to 0.05% (V/V) (diluting solution). The samples were tested by making serial dilutions to the medium so that they could be compared to the standard in the linearity range of the curve. It was incubated for 1 hour at 37° C. A check was performed without the addition of IFN-2b, to evaluate the possible non-specific binding of the reagents (negative control). To do this, during this stage the IFN was replaced with 100 L of diluent solution.

The second incubation was performed by adding 100 l of rabbit serum C7 anti-IFN-2b diluted 1:1,000 with diluent solution. It was incubated for 1 hour at 37° C.

The third incubation was performed by adding 100 L of rabbit anti-immunoglobulin goat antibody conjugated with the enzyme peroxidase (DAKO, Denmark) was added in a dilution 1:2,000 dilution in diluent solution. It was incubated for 1 hour at 37° C.

For the revealing reaction, the reveal was made by enzymatic reaction using as substrate H₂O₂ 0.015 volumes diluted in sodium citrate/phosphate solution 50 mM, pH 5.3 (reveal solution), with the addition of o-phenylenediamine chromogen (OPD, Sigma) at a concentration of 0.5 mg·ml⁻¹. 100 L per well of said solution was placed and, after 15 minutes of incubation in darkness at room temperature, the appearance of color was observed because the enzyme catalyzed the reduction of the substrate with simultaneous oxidation of the chromogen. The reaction was stopped by the addition of 50 L of H₂OS₄ 2N and the color reading was performed at a 0.492 nm on a microtitulation plate reader (Labsystems Multiskan MCC/340, Finland).

For quantification, the absorbance values were plotted based on the concentrations of IFN-2b used as standard and the dilutions of the samples, both in logarithmic scale. The concentration of the samples was determined using the parallel straight test (D: Milano, F. (2001) Bachelors Thesis in Biotechnology: Design and validation of bioassays for in vitro biological assessment of drugs. Faculty of Biochemistry and Biological Sciences, UNL, Santa Fe, Argentina.). SDS-PAGE and western blotting. SDS-PAGE analysis was performed according to the standard method using 15% (w/v) polyacrylamide resolving gels and 5% (w/v) stacking gels. Proteins were transferred onto a polyvinylidene difluoride (PVDF) membrane (BioRad). Blots were blocked for 1 h with 5% (w/v) non-fat milk in Tris-buffered saline (TBS) and then probed with rabbit anti-rhIFN-α2b polyclonal antibodies. After 1 h, blots were incubated with the same peroxidase-conjugated described in the ELISA. Immunoreactive bands were visualized using an ECL™ Chemiluminescent Western Blotting Analysis System (GE Healthcare). Washes between steps were performed with TBS containing 0.05% (v/v) Tween 20 (TBS-T). Dilutions were prepared in TBS-T containing 0.5% (w/v) nonfat milk.

### Example 2. GMOP-IFN De-Immunized Variants: Production and Purification

GMOP-IFN variants were synthesized and cloned into third generation lentiviral vectors and then expressed in CHO cells. After cell selection using puromycin (300 μg/ml), culture supernatants from stable cell lines were preliminary screened for rhIFN-α2b production and biological potency by sandwich ELISA and antiviral assays, respectively.

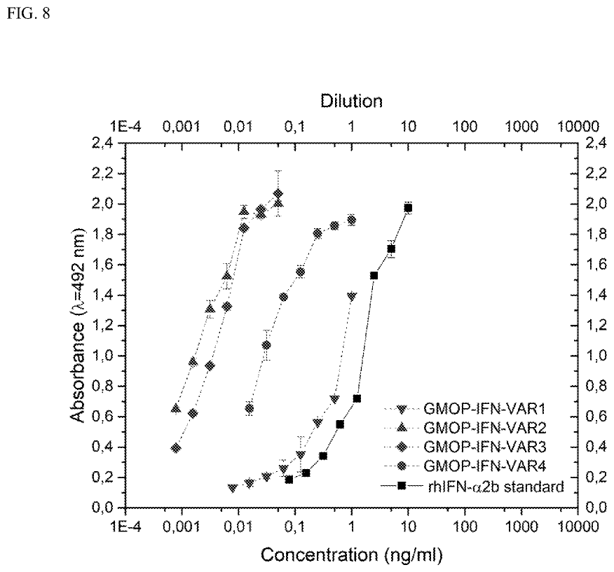

For protein purification, a one-step immune-affinity chromatography was performed using a monoclonal antibody (CA5E6), adsorbed on CNBr-activated Sepharose as ligand. Supernatants-containing protein variants were loaded onto the matrix, without exceeding 40% of its theoretical capacity. No loss of the cytokine was observed, neither in flow-through nor washing steps. Protein concentration was measured by spectrophotometric absorbance at a wavelength of λ=280 nm (FIG. 8).

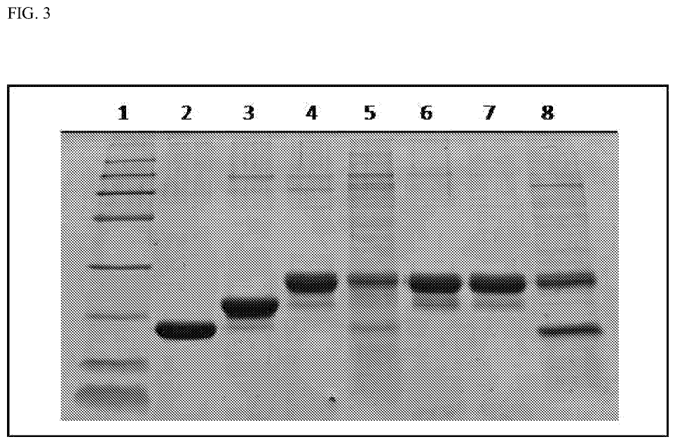

Protein purity was analyzed by SDS-PAGE followed by coomasie blue staining (FIG. 3). All protein preparations exhibited a similar mobility shift in SDS-PAGE. However, non-glycosylated rhIFN-α2b for all the samples was also detected, reflecting the presence of less efficiently occupied O-glycosylation consensus sequences. GMOP-IFN-VAR2 and GMOP-IFN-VAR3 densitometry profiles revealed purity levels over 94%, with the presence of Bovine Serum Albumin (BSA) as the main contaminant. In contrast, the achieved purity level for GMOP-IFN-VAR1 and GMOP-IFN-VAR4 proteins was around 80%, which may be attributed to a lower protein binding to the CA5E6 mAb.

It should be clarified that the modifications/substitutions presented by the deimmunized variants of GMOP-IFN-2b correspond to those amino acids not involved in the biological structure or function of cytokine. That is, this experiment and these mutations can be performed on any of the modified IFN-α2 polypeptides (or related modified IFN-α2 compounds and compositions) as disclosed herein, for example including the following variants: IFN-α2b, GMOP-IFN-α2b, or any other variant of IFN-α2 (including IFN-α2a, GMOP-IFN-α2a, IFN-α2c, and GMOP-IFN-α2c) with or without one or more GMOP sequences attached.

### (3) In Vitro Activity Assays

Antiviral assay. Antiviral biological titration assays for interferons quantify the inhibitory activity that these cytokines exert on viral propagation or replication (Familletti G et al, (1981), Methods Enzymology 78: 387-394). The simplest and most convenient procedure is to measure the ability of interferon to protect susceptible cells from the cytopathic effect of a lytic virus for a range of concentrations of the cytokine.

The biological antiviral activity of rhIFN-α2b was determined by its ability to inhibit the cytopathic effect caused by vesicular stomatitis virus (VSV) on MDBK cells (Familletti P C et al., (1981), Methods Enzymol. 78: 387-394; Rubinstein S et al., (1981 J. Virol. 37: 755-8). To evaluate the impact of modifications on the anti-viral activity of the GMOP-IFN variants, MDBK cells were seeded into culture microtiter plates in growth medium [MEM supplemented with 10% SFB (V/V)] (2.5×10⁴ cells per well) and incubated at 37° C. overnight.

After removing culture supernatants, 1:2 serial dilutions of rhIFN-α2b WHO international standard (NIBSC 95/566) from 20 U ml⁻¹ to 0.16 U ml⁻¹ or 1:2 serial dilutions of GMOP-IFN variants test samples were added in assay medium. The plates were then incubated for 6 h at 37° C., and after removal of supernatants, the monolayers were infected with 1.6 PFU of VSV virus per cell. Viral replication was allowed to proceed until the cytopathic effect was clearly observable in control wells (no rhIFN-α2b). The medium was discarded and cells were fixed and stained simultaneously with a solution of 0.75% (w/v) crystal violet in 40% (v/v) methanol (Merck). After 15 min at 37° C., plates were washed with distilled water to remove the dye, and the fixed dye was solubilized in 20% (v/v) acetic acid. The plates were read at A=540 nm with a microtiter plate reader, which allows homogenization of the plate prior to the reading, and the signal intensity of each dilution was reported as the mean of the absorbance measured in five wells.

The absorbance data were plotted as a function of the corresponding activity values of IFN-α2b (standard) and of the dilutions of the samples on a logarithmic scale and the biological activity values (AB) were calculated for each of the molecules by comparison, with the standard using the test of parallel lines. From these results and making the quotient between the AB and the concentration of the molecules in the samples, the values of specific biological activity (ABE) of each protein were determined.

Finally, the percentage relative antiviral activity value was determined by making the quotient between the ABE of the IFN-α2b-WT molecule (180±50 IU/ng) and the corresponding ABE of each of the GMOP-IFN-α2b variants.

Antiproliferative assay. In order to measure rhIFN-α2b ability to inhibit cell growth, an in vitro bioassay using Daudi cells was carried out (Nederman T et al., (1990), Biologicals. 18: 29-34). Serial 1:2 dilutions of rhIFN-α2b WHO international standard from 50 U/ml to 0.02 U/ml or GMOP-IFN variants test samples were placed into microtiter plate wells. Then, previously washed Daudi cells were added (5×10³ cells per well) and plates were incubated at 37° C. for 96 h. Cell proliferation was determined using a CellTiter 96™ AQueous Non-Radioactive Cell Proliferation Assay (Promega), which consists of two reagents: MTS [3-(4.5-dimethylthiazole-2-il)-5-(3-carboxymethoxy-phenyl)-2-(4-sulfophenyl)-2H-tetrazolium)] at a concentration of 2 mg·ml⁻¹ and PMS [phenazine methosulfate] at a concentration of 0.92 mg·ml⁻¹. The WHO international standard: rhIFN-2b produced in E. coli (NIBSC 95/566) was used.

In sterile flat-bottomed plates of 96 wells, 50 μl was placed per successive dilution well 1:2 of the rhIFN-α2b standard in RPMI medium supplemented with 10% SFB (V/N) (growth medium), from an activity concentration of 50 UI·ml⁻¹ to 0.39 UI·ml⁻¹. The same conditions were used to analyze the different proteins, making an appropriate initial dilution for each of them, such as to compare them with the standard in the linearity range of the dose-response curve.

The Daudi cell line was cultivated in the midst of growth. For the test, a suspension of 1.10⁵ cell ml⁻¹ was prepared, of which 50 μl was added in each well, incubating for 96 hours in stove at 37° C.

To reveal the test, 20 μl was added per well of the color reagent, prepared at the time by mixing 2 mL of MTS solution with 100 L of PMS solution per plate. It was incubated for 5 hours at 37° C. This colorimetric method measures cell proliferation, highlighting the presence of dehydrogenase enzymes found in metabolically active cells whose activity is directly related to the number of viable cells present in the culture. Dehydrogenase enzymes catalyze MTS bioreduction into a soluble (blue formazan) chromogen that absorbs at a wavelength of 490 nm. PmS acts as an electron giver in the oxide-reduction reaction. The amount of product generated is directly proportional to the number of metabolically active cells in the crop. The absorbance of the chromogen was measured at a 492 nm using a microplate reader against plate background reading at 690 nm. The assay was reproduced in triplicates.

Absorbance values were plotted based on the corresponding standard activity data and logarithmic scale sample dilutions. The antiproliferative biological activity values for each of the new molecules were calculated using the standard using the parallel line comparison method.

Finally, the specific antiproliferative biological activity value was determined by making the ratio between volumetric activity and protein concentration.

### Example 3A. GMOP-IFN-VAR2 and GMOP-IFN-VAR3 Exhibited High Residual Antiviral Activity and Null Antiproliferative Properties

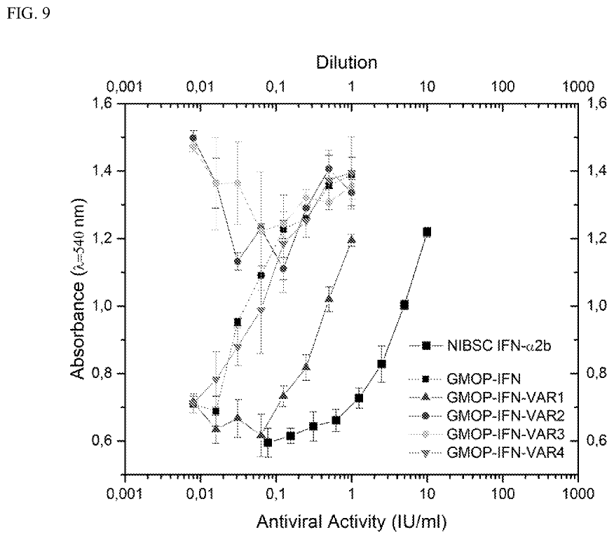

A deimmunization strategy was used with the aim to change the most immunogenic amino acids without altering those residues directly involved in antiviral activity. The impact of those modifications on cytokine's biological activity was evaluated by in vitro antiviral activity assays. MDBK cells were used as targets for viral infection by VSV virus, as this is the assay recommended by the European Pharmacopeia. Relative antiviral activity of the GMOP-IFN-2b variants with respect to GMOP-IFN-α2b (190±50 UI/ml) was determined by their ability to inhibit the cytopathic effect caused by vesicular stomatitis virus on MDBK cells and normalized to the activity of GMOP-IFN-α2b (FIGS. 9, 10, and 11). A preliminary antiviral activity test was performed using cell culture supernatants of production lines of each variant of GMOP-IFN-α2b. All the supernatants showed antiviral activity at different magnitudes (FIG. 9).

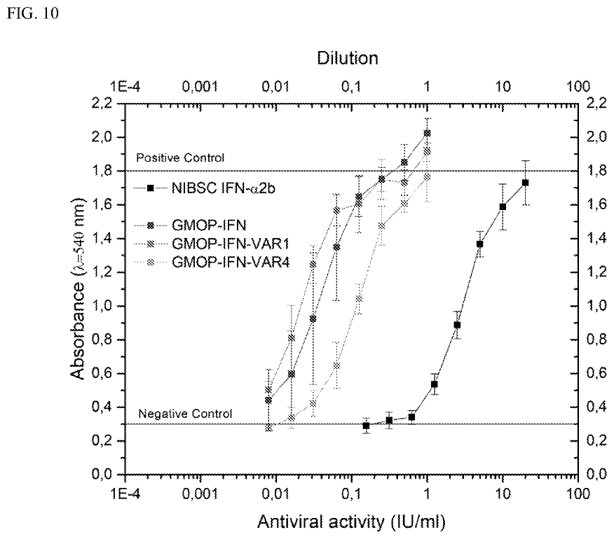

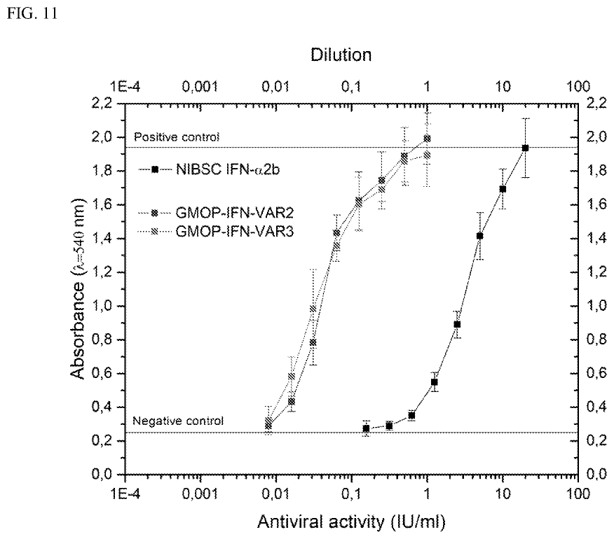

The percentage relative antiviral activity of the GMOP-IFN-α2b variants with respect to GMOP-IFN-α2b (190±50 UI/ml) was then determined, as described above, using purified GMOP-IFN-α2b and GMOP-IFN-α2b variants (FIGS. 10 and 11). A marked decrease in residual antiviral activity was observed for GMOP-IFN-VAR1 and GMOP-IFN-VAR4 (0.06% and 0.17%, respectively) (FIG. 10). Consequently, both proteins were discarded from further study. In contrast, as shown in Table 2, GMOP-IFN-VAR2 and GMOP-IFN-VAR3 retained most of the original antiviral activity (72% and 35%, respectively) (FIG. 11). This reflects that, in despite of restricting the selection of immunogenic residues to those not directly involved in biological activity, a partial reduction in the IFN-receptor interaction was still evident.

TABLE 2
GMOP-IFN-VAR2 and GMOP-IFN-VAR3 retained high residual
antiviral activity

| GMOP-IFN- | GMOP-IFN- | GMOP-IFN- | GMOP-IFN- |
|---|---|---|---|
| VAR1 | VAR2 | VAR3 | VAR4 |
| 0.06% ± 0.02% | 72% ± 4% | 35% ± 2% | 0.17% ± 0.05% |

During antiviral therapy with rhIFN-α, one of the most common side effects is the decrease in neutrophil counts or neutropenia, which is frequently associated with dose adjustment or early discontinuation (Saleh M I and Hindi N N, (2018), Naunyn. Schmiedebergs. Arch. Pharmacol. 391: 953-963). For this, to further characterize the antiproliferative activity of GMOP-IFN-VAR2 and GMOP-IFN-VAR3, an in vitro bioassay was used to measure their ability to inhibit cell growth of Daudi cells. In despite of not altering any residue directly involved in protein biological activity, a marked decrease of their specific antiproliferative activity was observed for both protein variants. As shown in Table 3, both GMOP-IFN-VAR2 and GMOP-IFN-VAR3 exhibited less than 1% of the original antiproliferative potency (0.5±0.2 Ung⁻¹ for GMOP-IFN-VAR2 and 0.4±0.1 Ung⁻¹ for and GMOP-IFN-VAR3). Taking these results altogether and given that the same cell receptor is involved in both hIFN-α2b biological activities, this denotes a greater susceptibility of the IFN antiproliferative activity to changes in the cytokine structure. These results are extremely positive, considering that high antiproliferative activity is generally associated with unwanted side effects of IFN-2b therapy, such as neutrocytopenia that generates susceptibility to serious infections (e.g., bacterial, viral and fungal).

TABLE 3
IFN-GMOP-VAR2 and 3 exhibited null antiproliferative properties.
Results are shown as percentage of residual antiproliferative activity
considering GMOP-IFN (280 ± 70 UI/ng) as reference value.

| Protein | GMOP-IFN-VAR2 | GMOP-IFN-VAR3 |
|---|---|---|
| Antiproliferative activity | 0.5 ± 0.04 | 0.4 ± 0.1 |
| (UI/ng) | | |

It should be clarified that the modifications/substitutions presented by the deimmunized variants of GMOP-IFN-2b correspond to those amino acids not involved in the biological structure or function of cytokine. That is, this experiment and these mutations can be performed on any of the modified IFN-α2 polypeptides (or related modified IFN-α2 compounds and compositions) as disclosed herein, for example including the following variants: IFN-α2b, GMOP-IFN-α2b, or any other variant of IFN-α2 (including IFN-α2a, GMOP-IFN-α2a, IFN-α2c, and GMOP-IFN-α2c) with or without one or more GMOP sequences attached.

### Example 3B. Comparative Residual Antiviral Activity and Antiproliferative Properties of IFN-α Variants as Compared to Other Interferons

The impact of various modifications on IFN-α2b cytokine's biological activity is evaluated by in vitro antiviral activity assays. MDBK cells are used as targets for viral infection by VSV virus, as this is the assay recommended by the European Pharmacopeia. Relative antiviral activity of hyperglycosylated GMOP-IFN variants 1-4 (of SEQ ID NOS: 2, 4, 6, and 8, respectively) with respect to GMOP-IFN-α2b (190±50 UI/ml) is determined by their ability to inhibit the cytopathic effect caused by vesicular stomatitis virus on MDBK cells and is normalized to the activity of GMOP-IFN-α2b.

Several other purified IFN-α2b variants are generated as well, in order to compare their biological activity with the hyperglycosylated GMOP-IFN variants. These variants include: PEGylated IFN-α2b, non-glycosylated IFN-α2b, non-glycosylated GMOP-IFN variants 1-4 (of SEQ ID NOS: 2, 4, 6, and 8, respectively), and 4N-IFN. 4N-IFN is a hyperglycosylated IFN-α2b variant, wherein mutations are introduced into natural hIFN-α2b by substituting an amino acid with Asn to provide consensus N-glycosylation sites consisting of an Asn-Xaa-Ser/Thr tripeptide, where X may be any residue except a proline residue. The 4N includes mutations to Asn at the following positions of hIFN-α2b: 4, 23, 70, and 77.

A decrease in residual antiviral activity is expected for hyperglycosylated GMOP-IFN-VAR1 and hyperglycosylated GMOP-IFN-VAR4. Additionally, a decrease in residual antiviral activity is expected for PEGylated IFN-α2b, non-glycosylated IFN-α2b, non-glycosylated GMOP-IFN variants 1-4, and 4N-IFN. In contrast, hyperglycosylated GMOP-IFN-VAR2 and hyperglycosylated GMOP-IFN-VAR3 retain or are expected to retain most of the original antiviral activity. This reflects that, in despite of restricting the selection of immunogenic residues to those not directly involved in biological activity, a partial reduction in the IFN-receptor interaction is still evident.

During antiviral therapy with rhIFN-α, one of the most common side effects is the decrease in neutrophil counts or neutropenia, which is frequently associated with dose adjustment or early discontinuation (Saleh M I and Hindi N N, (2018), Naunyn. Schmiedebergs. Arch. Pharmacol. 391: 953-963). For this, to further compare the antiproliferative activity of hyperglycosylated GMOP-IFN-VAR2 and hyperglycosylated GMOP-IFN-VAR3 with PEGylated IFN-α2b, non-glycosylated IFN-α2b, non-glycosylated GMOP-IFN variants 1-4, and 4N-IFN, an in vitro bioassay is used to measure their ability to inhibit cell growth of Daudi cells. In despite of not altering any residue directly involved in protein biological activity, a marked decrease of their specific antiproliferative activity is observed for both of hyperglycosylated GMOP-IFN-VAR2 and hyperglycosylated GMOP-IFN-VAR3. Conversely, the specific antiproliferative activity is not expected to be significantly reduced for PEGylated IFN-α2b, non-glycosylated IFN-α2b, non-glycosylated GMOP-IFN variants 1-4, and 4N-IFN. Taking these results altogether and given that the same cell receptor is involved in both hIFN-α2b biological activities, this denotes a greater susceptibility of the IFN antiproliferative activity to changes in the cytokine structure. These results are extremely positive, considering that high antiproliferative activity is generally associated with unwanted side effects of IFN-2b therapy, such as neutrocytopenia that generates susceptibility to serious infections (e.g., bacterial, viral and fungal).

It should be clarified that the modifications/substitutions presented by the deimmunized variants of GMOP-IFN-2b correspond to those amino acids not involved in the biological structure or function of cytokine. That is, this experiment and these mutations can be performed on any of the modified IFN-α2 polypeptides (or related modified IFN-α2 compounds and compositions) as disclosed herein, for example including the following variants: IFN-α2b, GMOP-IFN-α2b, or any other variant of IFN-α2 (including IFN-α2a, GMOP-IFN-α2a, IFN-α2c, and GMOP-IFN-α2c) with or without one or more GMOP sequences attached.

### (4) Physiochemical Characterization

Isoelectric focusing. IEF was performed in 1 mm thick 8% (w/v) polyacrylamide gels containing 7 M urea, 30% (w/v) 5-7 ampholytes and 70% (w/v) 2-4 ampholytes (Pharmalyte, GE Healthcare), mixed to establish the pH range. The gel was prefocused at 10W, 2000V and 100 mA for 30 min. Then, 5-20 μl samples were applied at 1 cm from cathode and electrophoresis was carried out using the same conditions as the prefocusing step for 90 min. The IEF-separated components were detected by Coomasie blue staining.
Evaluation of suitable O-glycosylation sites in GMOP-IFN and its variants. Given the lack of known consensus recognition sequences for the O-glycosyltransferases, neural network predictions of mucin type GalNAc O-glycosylation sites were performed by using the NetOGlyc 3.1 Server software (Julenius K et al., (2005), Glycobiology. 15: 153-164).

### Example 4. GMOP-IFN Deimmunized Variants Showed Characteristic Electrophoretic Profiles

To further characterize the charge-based heterogeneity for each protein variant, an isoelectric focusing (IEF) assay was performed. For WT-IFN, rhIFN-α2b produced in CHO-K1 cells, four electrophoretic bands were observed that represent isoforms with O-glycan structures carrying different content of sialic acid attached to the natural Thr106 O-glycosylation site.

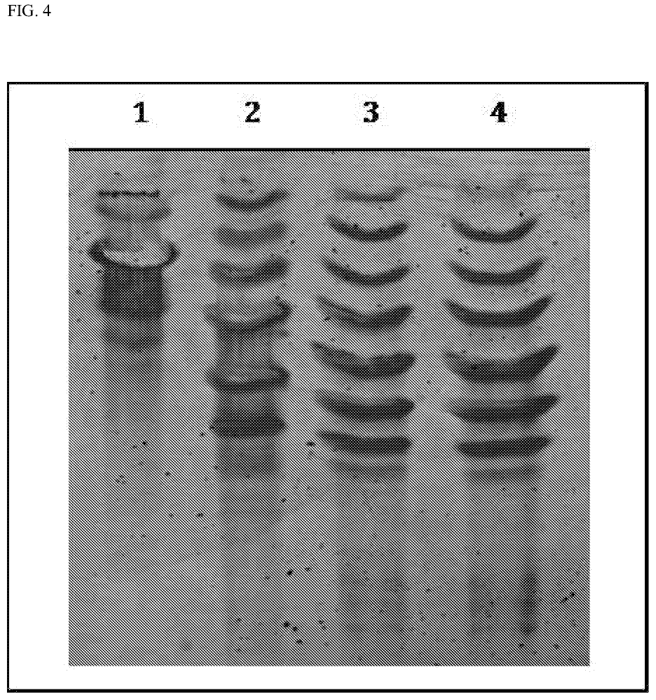

A higher content of glycan structures bound to the O-glycosylation moieties of GMOP-IFN were evidenced by the presence of approximately 7 isoforms, situated in the most acidic region of the gel. Interestingly, both deimmunized variants showed a different electrophoretic profile when compared with the original molecule. A total of 11 electrophoretic bands were detected for both proteins and three of them were located at the most acidic end of the gel. Moreover, a lower content of the most basic isoform for GMOP-IFN-VAR3 (FIG. 4) was also observed. These results are in agreement with data from the analysis of mucin type GalNAc O-glycosylation sites using the NetOGlyc 3.1 server software. This algorithm predicted the occurrence of five O-glycosylation sites for GMOP-IFN and six for GMOP-IFN-VAR2 and GMOP-IFN-VAR3.

It should be clarified that the modifications/substitutions presented by the deimmunized variants of GMOP-IFN-2b correspond to those amino acids not involved in the biological structure or function of cytokine. That is, this experiment and these mutations can be performed on any of the modified IFN-α2 polypeptides (or related modified IFN-α2 compounds and compositions) as disclosed herein, for example including the following variants: IFN-α2b, GMOP-IFN-α2b, or any other variant of IFN-α2 (including IFN-α2a, GMOP-IFN-α2a, IFN-α2c, and GMOP-IFN-α2c) with or without one or more GMOP sequences attached.

### (5) Immunogenicity Assessment

Human PBMC preparation and HLA-DR typing. All blood extraction and handling procedures were approved by the Universidad Nacional del Litoral Research Ethics Committee (Santa Fe, Ark.). Blood samples were obtained from healthy donors, aged between 18 and 60 years, by venipuncture after obtaining informed consent.

PBMCs were isolated by Ficoll-Paque™ PLUS (GE Healthcare Bio-Science, SE) density gradient separation according to manufacturers instructions, and the buffy coat was collected and washed twice with PBS. PBMCs were cryopreserved in liquid nitrogen at a concentration of 1-3×10⁷ cells/ml. Previously, an aliquot of blood was separated and HLA-DR allotypes were determined by Luminex Sequencing Technology (PRICAI, Buenos Aires, Ark.). Typing results were compared to publicly available HLA-DR frequencies in the world population on the Allele Frequency Net Database (The Royal Liverpool and Broadgreen University Hospitals, NHS Trust website: www.allelefrequencies.net).

Ex vivo T-cell assays. For ex vivo T-cell assays a strategy suggested by Jaber and Baker ((2007), J. Pharm. Biomed. Anal. 43: 1256-1261) with modifications was performed. Monocytes were isolated from PBMCs from each donor blood sample by differential adherence to plastic (Elkord E et al., (2005), Immunology. 114: 204-12). The adherent cells were retained for differentiation and the non-adherent cells were collected and cryopreserved for further use. To induce an immature phenotype of monocyte-derived DC, monocytes were incubated in growth medium containing 1000 U/ml each of human IL-4 (Millipore, USA) and granulocyte macrophage colony stimulating factor (GM-CSF, GemaBiotech, AR) for 6 days with a change of media at day 3. On day 6, immature dendritic cells were collected, counted and incubated with rhIFN-α2b variants or non-antigen (medium or excipients). Test antigens included in this study were GMOP-IFN and its de-immunized variants. After an overnight incubation, DC were washed to remove exogenous antigen, and resuspended in growth medium containing recombinant human tumor necrosis factor (rhTNF, ProsPec, USA) alpha, GM-CSF and IL-4 for 4 days, to induce DC maturation. Ag pulsed-DCs were then incubated with autologous cells for 48 h in medium containing 2 ng/ml human IL-2 (Thermo, USA). Supernatants were collected and evaluated for IFN-γ and IL-4 quantification by sandwich ELISA. Negative controls (medium or excipients), and positive controls (phytohemagglutinin, Sigma Aldrich, USA) were also included.
IFN-α sandwich ELISA. 96-well plates were coated with 100 μl primary hIFN-γ mAb (clone NIB42, BD, USA) at a concentration of 2 μg/ml, first for 1 h at 37° C. and then overnight at 4° C. After blocking 1 h at 37° C. with 1% (w/v) BSA in phosphate-buffered saline (PBS), culture supernatants were added and incubated for 2 h at 37° C. Serial 1:2 dilutions of rhIFN-γ (BD, USA) from 1 ng/ml were also included. Then, 100 μl/well of biotinylated hIFN-γ mAb (clone 4S.B3, BD, USA) at a concentration of 500 ng ml⁻¹ was added to the plates and incubated for 1 h at 37° C. Then, plates were incubated with Streptavidin horseradish peroxidase conjugate (RPN4401-AMDEX, USA) diluted 1:5000. After 1 h, plates were incubated with substrate solution (0.5 mg ml-1 o-phenylenediamine, 0.015% (v/v) H₂O₂ in 50 mM phosphate citrate buffer). Reactions were stopped by the addition of 2N H₂SO₄ and the absorbance was measured at 492 nm with a microtiter plate reader Labsystems Multiskan MCC/340, Finland). Between every step, plates were washed with PBS containing 0.05% (v/v) Tween 20 (PBS-T). Dilutions were prepared in PBS-T containing 0.1% (w/v) BSA. The assay was performed in triplicate. The Stimulation Index (SI) was defined as a ratio of the cytokine concentration from protein challenged samples divided by cytokine concentration from excipient treated samples.
Statistical Analysis. Differences between treatments were evaluated through a one-way analysis of variance (ANOVA). When the ANOVA produced significant differences (p<0.05), a post-hoc Tukey's multiple comparison test was applied.

### Example 5. Immunogenicity Analysis

Ex vivo human PBMC assays are based on measuring immune cell activation after exposure to therapeutic candidates. These allow to analyze the antigen-specific activation of T cells and determine the induction potential of the immune response presented by the therapeutic. The composition of these samples include not only some relevant immune cells such as T lymphocytes but also antigen presenting cells (e.g. monocytes, dendritic cells and B cells). If, as a result of this exposure to the therapeutic, an immune response occurs, it can be measured by quantifying certain cytokines, secreted by activated collaborating T cells, such as IFN-γ, IL-4, IL-6, TNF-α, among others. Consequently, this constitutes a suitable experimental platform to evaluate the risk associated with the presence of potentially immunogenic T-cell epitopes in therapeutic proteins.

Donors samples. Human immune cell based assays have been extensively used as protein immunogenicity risk assessments (Jaber A and Baker M, (2007), J. Pharm. Biomed. Anal. 43:1256-1261; Mazor R et al, (2012), Proc. Natl. Acad. Sci. U.S.A. 109: E3597-603; Lamberth K et al., (2017), Sci. Transl. Med. 9: 1-12). It is very clear now that these experiments are more reliable when carried out with diverse HLA genotypes donor pools and representative of the HLA occurrence in the world-wide population. In this study, blood samples were collected from 20 healthy donors aged between 18 and 60 years. An aliquot of blood was taken from each donor and HLA-DR allotypes were determined by Luminex Sequencing Technology. Briefly, this technique consists of a PCR (Polymerase Chain Reaction) amplification of extract 2 of the DRB1 gene, and then hybridization with specific probes that are attached to polystyrene spheres marked with orochromes. These spheres are read by the Luminex team and detected if the PCR product hybridized to the traces attached to the spheres. HLA-DRB1 alleles expressed by donors exhibited high heterogeneity and are shown in Table 4.

TABLE 4
HLA-DRB1 alleles expressed by donors exhibited high heterogeneity.5

| | Allele | |
|---|---|---|
| Donor | DRB1_1 | DRB1_2 |
| 1 | DRB1*01 | DRB1*04 |
| 2 | DRB1*15 | DRB1*15 |
| 3 | DRB1*04 | DRB1*13 |
| 4 | DRB1*03 | DRB1*04 |
| 6 | DRB1*03 | DRB1*08 |
| 7 | DRB1*01 | DRB1*03 |
| 8 | DRB1*09 | DRB1*11 |
| 9 | DRB1*11 | DRB1*16 |
| 10 | DRB1*07 | DRB1*13 |
| 12 | DRB1*03 | DRB1*08 |
| 13 | DRB1*07 | DRB1*11 |
| 14 | DRB1*01 | DRB1*13 |
| 16 | DRB1*11 | DRB1*15 |
| 17 | DRB1*01 | DRB1*16 |
| 18 | DRB1*04 | DRB1*15 |
| 19 | DRB1*11 | DRB1*15 |
| 22 | DRB1*01 | DRB1*03 |
| 23 | DRB1*04 | DRB1*13 |
| 24 | DRB1*07 | DRB1*11 |
| 25 | DRB1*13 | DRB1*16 |

T-cell activation response. The endogenous hIFN-α2b antiproliferative effect on T-cells restricts its direct incubation with PBMC samples. To circumvent this issue, an alternative protocol was adapted that included a previous step for generation of monocyte-derived DC (moDC). Immature DCs were pulsed with the different GMOP-IFN variants during short incubation time, at a high concentration, and then the cells were washed. During this step, immature DCs are able to endocytose and process the antigen. Upon maturation, DCs can present GMOP-IFN-derived peptides bound to MHC class II on the cell surface, where they would be available to stimulate T-cell responses. Blood samples were obtained from healthy donors and selected so as to include most major HLA-DR allotypes expressed in the world population. This enables the detection of any hIFN-α2b specific T-cell responses restricted to a particular HLA-DR allotype. Ex vivo T-cell assays and IFN-γ sandwich ELISAs were performed to evaluate the concentrations of IFN-γ and IL-4, as described above. The concentrations of these cytokines in the culture supernatants of the incubated samples with the proteins to be analyzed were compared with the levels of negative controls (dendritic cells incubated with PBS or excipients and faced with lymphocytes). Finally, the stimulation rates were calculated from the ratio between the IFN-γ levels in the sample with respect to negative control. From there, the percentage of donors who had reduced levels of IFN-γ in supernatant was assessed for each variant, with respect to the original GMOP-IFN-2b, considering significant differences between samples when p≤0.05.

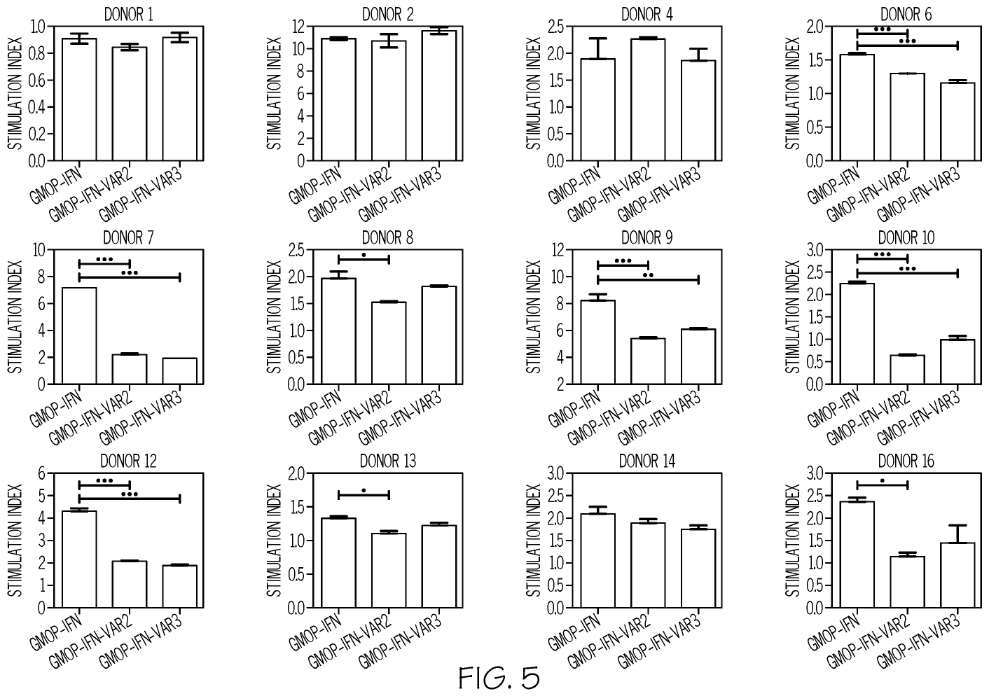

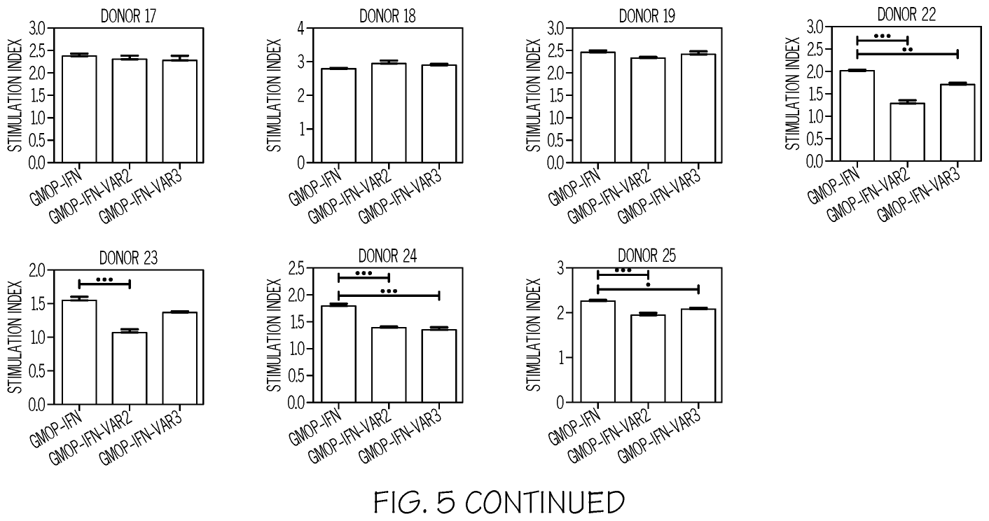

As shown in FIG. 5, almost all the donors responded to GMOP-IFN protein, as judged by an increase in IFN-γ production when compared to the negative control. This result is in good agreement with the computational predictions. Also consistent with the findings using the EpiMatrix algorithm, a marked reduction in immunogenicity was observed for both GMOP-IFN deimmunized variants, as evidenced by a reduction of the percentage of IFN-γ responses in 63% of donors for GMOP-IFN-VAR2 and 42% for GMOP-IFN-VAR3.

However, when analyzing IL-4 secretion (Th2 profile) no measurable levels of the cytokine were detected, with the exception of cells incubated with lectins that showed a clear T-cell activation.

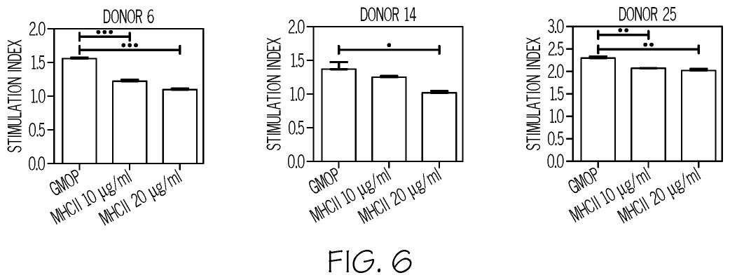

HLA-DR restriction for Antigen Presentation. To confirm that antigen presentation was mediated in the context of HLA-DR molecules, GMOP-IFN-pulsed dendritic cells derived from three responsive donors were treated with an anti-DR antibody (in two different concentrations) before incubation with autologous T-cells. A lower T-cell activation, as judged by a reduction in IFN-γ Stimulation Index (SI), was observed when DCs were previously treated with the anti-DR antibody (FIG. 6). Moreover, this effect was even more pronounced when the added amount of antibody was increased, demonstrating the essential role of HLA-DR molecules for IFN-derived peptide presentation and consequent T-cell activation.

It should be clarified that the modifications/substitutions presented by the deimmunized variants of GMOP-IFN-2b correspond to those amino acids not involved in the biological structure or function of cytokine. That is, this experiment and these mutations can be performed on any of the modified IFN-α2 polypeptides (or related modified IFN-α2 compounds and compositions) as disclosed herein, for example including the following variants: IFN-α2b, GMOP-IFN-α2b, or any other variant of IFN-α2 (including IFN-α2a, GMOP-IFN-α2a, IFN-α2c, and GMOP-IFN-α2c) with or without one or more GMOP sequences attached.

### (6) Pharmacokinetic Profiles

The evaluation of the pharmacokinetics of a biopharmaceutical by determining its biological activity provides valuable information as it allows the specific quantification of the protein fraction that is active in the sampled biological fluid.

### Example 6A. Comparative Pharmacokinetic Profiles of IFN-α Variants in Rats

In order to evaluate the effect of de-immunizing mutations introduced into the GMOP-IFN-α2b sequence on in vivo protein properties, pharmacokinetic parameters for GMOP-IFN-α2b and its de-immunized variants were analyzed.

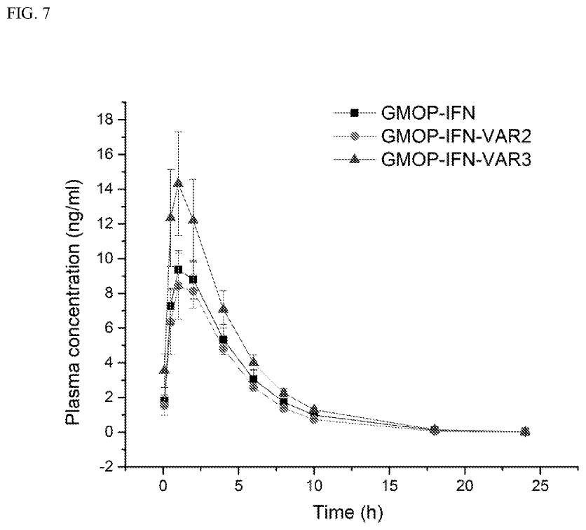

Female Wistar rats, two months old, with an average weight of 200 g (Center for Biological Experimentations and Bioterio, FCV-UNL), were used, which were kept in a biorium at a controlled temperature of 24° C. and a light/dark photoperiod of 12 hours, providing them with unrestricted water and food. The rats were separated into batches of eight animals each and subcutaneously inoculated with a single dose (in the same mass units) of GMOP-IFN-α2b, GMOP-IFN-α2b(VAR2) or GMOP-IFN-α2b(VAR3). The presence of IFN-α in rat plasma samples was monitored by collecting blood samples at different post-injection times by evaluating the remaining antiviral biological activity. The samples were centrifuged at 100×g for 10 min at room temperature and the plasma was separated and preserved at −20° C. for further analysis. Then, plasma protein concentration was plotted versus time (FIG. 7).

The behavior of proteins studied after subcutaneous inoculation showed absorption and elimination processes that can be assumed as first-order processes. For this reason, to describe the behavior of cytokines, a mathematical model was worked on in which both the overall rate of absorption and the rate of elimination can be treated as first-order processes. In this way, the experimental data were adjusted to a curve that allowed for calculation of the constants that characterize it and, finally, determine the pharmacokinetic parameters shown in Table 5.

Data was analyzed by using a one-compartment model, assuming first-order absorption and elimination kinetics. Pharmacokinetic parameters considered here were: maximum plasma protein concentration (Cₘₐₓ); time required to reach maximum plasma protein concentration (Tₘₐₓ); terminal half-life time (t_{1/2}), which refers to the time at which plasma protein concentration is 50% of the initial value; and apparent plasma clearance (Clₐₚₚ), which is the drug clearance rate (without considering drug bioavailability in the rat body). Differences between treatments were evaluated by ANOVA (p≤0.05) followed by Tukey's test.

As shown in Table 5, all IFN-α2 variants exhibited similar absorption and distribution phases, with no significant differences between them. No significant differences were shown in the times when each protein analogue achieved maximum biological activity in plasma (Tₘₐₓ), indicating that the initial distribution phase of cytokines would be similar, above the max Tᵥₐₗᵤₑ recorded for cytokine wild type (0.6±0.3 h).

Regarding the elimination phase, no differences in t_{1/2} were detected between IFN-α2 variants, all of which were much higher than the one described for IFN-2b-WT (0.9±0.2 h).

However, a significant reduction in plasma clearance rate (Clₐₚₚ) for GMOP-IFN-α2b-VAR3 in comparison with GMOP-IFN-α2b was observed. The differences between these proteins could be related to the diversity of the glycosydic structures attached to them, evidenced in the isoelectrofocus assay (FIG. 4).

In conclusion, altogether these results demonstrate that the improved pharmacokinetic properties obtained as a consequence of carbohydrate-rich peptide attachment to IFN-α2b molecule were retained for the de-immunized variants. Moreover, a further plasma clearance rate improvement was detected for GMOP-IFN-α2b-VAR3.

TABLE 5
IFN-α2 variants pharmacokinetic parameters in rats after
subcutaneous injection.

| | Parameter | | | | |
|---|---|---|---|---|---|
| Protein | Tₘₐₓ (h) | Cₘₐₓ (ng/ml) | t_{1/2} (h) | Clₐₚₚ (ml/h) | |
| GMOP-IFNα | 1.3 ± 0.2 | 10 ± 1 | 2.4 ± 0.1 | 124 | ± 18 |
| GMOP-IFNα-VAR2 | 1.4 ± 0.3 | 9 ± 2 | 2.2 ± 0.1 | 116 | ± 10 |
| GMOP-IFNα-VAR3 | 1.0 ± 0.2 | 14 ± 3 | 2.5 ± 0.3 | 73 | ± 9 * |

Asterisk character (*) denotes significant differences (p < 0.05) between the values of the indicated parameter for GMOP-IFN and GMOP-IFN-VAR3.

It should be clarified that the modifications/substitutions presented by the deimmunized variants of GMOP-IFN-2b correspond to those amino acids not involved in the biological structure or function of cytokine. That is, this experiment and these mutations can be performed on any of the modified IFN-α2 polypeptides (or related modified IFN-α2 compounds and compositions) as disclosed herein, for example including the following variants: IFN-α2b, GMOP-IFN-α2b, or any other variant of IFN-α2 (including IFN-α2a, GMOP-IFN-α2a, IFN-α2c, and GMOP-IFN-α2c) with or without one or more GMOP sequences attached.

### Example 6B. Comparative Pharmacokinetic Profiles of IFN-α Variants in Rats as Compared to Other Interferons

In order to evaluate the effect of de-immunizing mutations introduced into the GMOP-IFN-α2b sequence on in vivo protein properties, pharmacokinetic parameters for hyperglycosylated GMOP-IFN-α2b and its hyperglycosylated de-immunized variants are analyzed. The pharmacokinetic parameters for PEGylated IFN-α2b, non-glycosylated IFN-α2b, non-glycosylated GMOP-IFN variants 1-4 (of SEQ ID NOS: 2, 4, 6, and 8, respectively), non-glycosylated GMOP-IFN-α2b, and 4N-IFN are also analyzed.

Female Wistar rats, two months old, with an average weight of 200 g (Center for Biological Experimentations and Bioterio, FCV-UNL), are used, which are kept in a biorium at a controlled temperature of 24° C. and a light/dark photoperiod of 12 hours, providing them with unrestricted water and food. The rats were separated into batches of eight animals each and subcutaneously inoculated with a single dose (in the same mass units) of hyperglycosylated GMOP-IFN-α2b, hyperglycosylated GMOP-IFN-α2b(VAR2), hyperglycosylated GMOP-IFN-α2b(VAR3), PEGylated IFN-α2b, non-glycosylated IFN-α2b, non-glycosylated GMOP-IFN variants 1-4, non-glycosylated GMOP-IFN-α2b, and 4N-IFN. The presence of IFN-α in rat plasma samples is monitored by collecting blood samples at different post-injection times by evaluating the remaining antiviral biological activity. The samples are centrifuged at 100×g for 10 min at room temperature and the plasma is separated and preserved at −20° C. for further analysis. Then, plasma protein concentration is plotted versus time.

The quantification of proteins in plasma is carried out by assessment of its biological activity. With the data obtained, the biological activity of each sample is plotted according to the time elapsed since the inoculation of the molecule. The behavior of proteins studied after subcutaneous inoculation shows absorption and elimination processes that can be assumed as first-order processes. For this reason, to describe the behavior of cytokines, a mathematical model is worked on in which both the overall rate of absorption and the rate of elimination can be treated as first-order processes. In this way, the experimental data are adjusted to a curve that allows for calculation of the constants that characterize it and, finally, determine the pharmacokinetic parameters.

Data is analyzed by using a one-compartment model, assuming first-order absorption and elimination kinetics. Pharmacokinetic parameters considered here are: maximum plasma protein concentration (Cₘₐₓ); time required to reach maximum plasma protein concentration (Tₘₐₓ); terminal half-life time (t_{1/2}), which refers to the time at which plasma protein concentration is 50% of the initial value; and apparent plasma clearance (Clₐₚₚ), which is the drug clearance rate (without considering drug bioavailability in the rat body). Differences between treatments are evaluated by ANOVA (p≤0.05) followed by Tukey's test.

Hyperglycosylated GMOP-IFN-α2b(VAR2) and hyperglycosylated GMOP-IFN-α2b(VAR3) exhibit similar absorption and distribution phases, with no significant differences between them. No significant differences are shown in the times when each protein analogue achieved maximum biological activity in plasma (Tₘₐₓ), indicating that the initial distribution phase of cytokines will be similar, above the max Tᵥₐₗᵤₑ that is recorded for cytokine wild type. Conversely, PEGylated IFN-α2b, non-glycosylated IFN-α2b, non-glycosylated GMOP-IFN variants 1-4, non-glycosylated GMOP-IFN-α2b, and 4N-IFN are expected to show diminished max Tᵥₐₗᵤₑ.

Regarding the elimination phase, no differences in t_{1/2} are detected Hyperglycosylated GMOP-IFN-α2b(VAR2) and hyperglycosylated GMOP-IFN-α2b(VAR3), both of which are much higher than the one described for IFN-2b-WT. Conversely, PEGylated IFN-α2b, non-glycosylated IFN-α2b, non-glycosylated GMOP-IFN variants 1-4, non-glycosylated GMOP-IFN-α2b, and 4N-IFN are expected to show significantly lower t_{1/2} than the one described for IFN-2b-WT

However, a significant reduction in plasma clearance rate (Clₐₚₚ) for GMOP-IFN-α2b-VAR3 in comparison with GMOP-IFN-α2b is observed. The differences between these proteins could be related to the diversity of the glycosydic structures attached to them. On the other hand, a high plasma clearance rate (Clₐₚₚ) is expected for PEGylated IFN-α2b, non-glycosylated IFN-α2b, non-glycosylated GMOP-IFN variants 1-4, non-glycosylated GMOP-IFN-α2b, and 4N-IFN.

In conclusion, altogether these results demonstrate that the improved pharmacokinetic properties that will be obtained as a consequence of carbohydrate-rich peptide attachment to IFN-α2b molecule will be retained for the de-immunized variants. Moreover, a further plasma clearance rate improvement was detected for GMOP-IFN-α2b-VAR3. Conversely, the improved pharmacokinetic properties of the de-immunized variants are not expected to be observed in PEGylated IFN-α2b, non-glycosylated IFN-α2b, non-glycosylated GMOP-IFN variants 1-4, non-glycosylated GMOP-IFN-α2b, and 4N-IFN.

It should be clarified that the modifications/substitutions presented by the deimmunized variants of GMOP-IFN-2b correspond to those amino acids not involved in the biological structure or function of cytokine. That is, this experiment and these mutations can be performed on any of the modified IFN-α2 polypeptides (or related modified IFN-α2 compounds and compositions) as disclosed herein, for example including the following variants: IFN-α2b, GMOP-IFN-α2b, or any other variant of IFN-α2 (including IFN-α2a, GMOP-IFN-α2a, IFN-α2c, and GMOP-IFN-α2c) with or without one or more GMOP sequences attached.

### EQUIVALENTS

While the instant disclosure has been described in connection with the specific aspects thereof, it will be understood that it is capable of further modification. Furthermore, this application is intended to cover any variations, uses, or adaptations of the invention, including such departures from the present disclosure as come within known or customary practice in the art to which the disclosure pertains, and as fall within the scope of the appended claims.

1-108. (canceled)

109. A modified interferon-α2 polypeptide having interferon-α2 activity, the polypeptide comprising:
an amino acid sequence comprising at least 80% identity to SEQ ID NO: 12 and comprising one or more amino acid substitutions in any of the positions selected from the group consisting of: 9, 17, 47, 65, 66, 117, 123, 128, 147, and 157; wherein said substitution comprises the change of the amino acid of said position to alanine, glycine, or threonine; or
an amino acid sequence with at least 80% homology to SEQ ID NO: 10 and comprising one or more amino acid substitutions in any of the positions selected from the group consisting of: 23, 31, 61, 79, 80, 131, 137, 142, 161, and 171; wherein said substitution comprises the change of the amino acid of said position to alanine, glycine, or threonine; or
wherein the polypeptide comprises an amino acid sequence with at least 80% homology to SEQ ID NO: 22 and comprising at least five amino acid substitutions in any of the positions selected from the group consisting of: 9, 17, 47, 65, 66, 117, 123, 128, 147, and 157; wherein said substitution comprises the change of the amino acid of said position to alanine, glycine, or threonine; or
wherein the polypeptide comprises an amino acid sequence with at least 80% homology to SEQ ID NO: 21 and comprising at least five amino acid substitutions in any of the positions selected from the group consisting of: 23, 31, 61, 79, 80, 131, 137, 142, 161, and 171; wherein said substitution comprises the change of the amino acid of said position to alanine, glycine, or threonine; or
wherein the polypeptide comprises an amino acid sequence with at least 80% homology to SEQ ID NO: 24 and comprising at least five amino acid substitutions in any of the positions selected from the group consisting of: 9, 17, 47, 65, 66, 117, 123, 128, 147, and 157; wherein said substitution comprises the change of the amino acid of said position to alanine, glycine, or threonine;
wherein the polypeptide comprises an amino acid sequence with at least 80% homology to SEQ ID NO: 23 and comprising at least five amino acid substitutions in any of the positions selected from the group consisting of: 23, 31, 61, 79, 80, 131, 137, 142, 161, and 171; wherein said substitution comprises the change of the amino acid of said position to alanine, glycine, or threonine;
a nucleic acid encoding any one of said polypeptides;
a plasmid encoding any one of said polypeptides;
a vector comprising a nucleic acid encoding any one of said polypeptides;
a cell line comprising a nucleic acid encoding any one of said polypeptides;
or a pharmaceutical composition comprising any one of said polypeptides.

110. The modified interferon-α2 polypeptide of claim 109, wherein the polypeptide has a percentage antiproliferative biological activity of less than 5%.

111. The modified interferon-α2 polypeptide of claim 109, wherein the polypeptide has an apparent plasma clearance rate (Clₐₚₚ) of less than 115 mL/h.

112. The modified interferon-α2 polypeptide of claim 109, wherein the polypeptide is hyperglycosylated.

113. The modified interferon-α2 polypeptide of claim 109, wherein said polypeptide comprises the amino acid sequence comprising at least 80% identity to SEQ ID NO: 12 and comprising mutations: L9A, F47A, L117A, F123A, and L128A.

114. The modified interferon-α2 polypeptide of claim 109 further comprising mutations:
I147T and L157A;
N65A and L66A;
L17A, I147T, and L157A; or
combinations thereof.

115. The modified interferon-α2 polypeptide of claim 109, wherein said polypeptide comprises the amino acid sequence with at least 80% homology to SEQ ID NO: 10 comprising mutations: L23A, F61A, L131A, F137A, and L142A.

116. The modified interferon-α2 polypeptide of claim 115 further comprising mutations:
I161T and L171A;
N79A and L80A;
L31A, I161T, and L171A; or
combinations thereof.

117. The modified interferon-α2 polypeptide of claim 109, wherein said polypeptide comprises the amino acid sequence with at least 80% homology to SEQ ID NO: 22, comprising mutations L9A, F47A, L117A, F123A, and L128A.

118. The modified interferon-α2 polypeptide of claim 117, and further comprising mutations:
I147T and L157A;
N65A and L66A;
L17A, I147T, and L157A; or
combinations thereof.

119. The modified interferon-α2 polypeptide of claim 109, wherein said polypeptide comprises the amino acid sequence with at least 80% homology to SEQ ID NO: 21, comprising mutations L23A, F61A, L131A, F137A, and L142A.

120. The modified interferon-α2 polypeptide of claim 119 further comprising mutations:
I161T and L171A;
N79A and L80A;
L31A, I161T, and L171A; or
combinations thereof.

121. The modified interferon-α2 polypeptide of claim 109, wherein said polypeptide comprises the amino acid sequence with at least 80% homology to SEQ ID NO: 24, comprising mutations L9A, F47A, L117A, F123A, and L128A.

122. The modified interferon-α2 polypeptide of claim 121 further comprising mutations:
I147T and L157A;
N65A and L66A;
L17A, I147T, and L157A; or
combinations thereof.

123. The modified interferon-α2 polypeptide of claim 109, wherein said polypeptide comprises the amino acid sequence with at least 80% homology to SEQ ID NO: 23, comprising mutations L23A, F61A, L131A, F137A, and L142A.

124. The modified interferon-α2 polypeptide of claim 123 further comprising mutations:
I161T and L171A;
N79A and L80A;
L31A, I161T, and L171A; or
combinations thereof.

125. The modified interferon-α2 polypeptide of claim 109, wherein
the polypeptide comprises the amino acid sequence with at least 80% homology to SEQ ID NO: 12 and the polypeptide has a reduced immunogenicity as compared to a wild type interferon-α2b polypeptide of SEQ ID NO: 12;
the polypeptide comprises the amino acid sequence with at least 80% homology to SEQ ID NO: 10 and the polypeptide has a reduced immunogenicity as compared to a wild type interferon-α2b polypeptide of SEQ ID NO: 10;
the polypeptide comprises the amino acid sequence with at least 80% homology to SEQ ID NO: 22 and the polypeptide has a reduced immunogenicity as compared to a wild type interferon-α2b polypeptide of SEQ ID NO: 22;
the polypeptide comprises the amino acid sequence with at least 80% homology to SEQ ID NO: 21 and the polypeptide has a reduced immunogenicity as compared to a wild type interferon-α2b polypeptide of SEQ ID NO: 21;
the polypeptide comprises the amino acid sequence with at least 80% homology to SEQ ID NO: 24 and the polypeptide has a reduced immunogenicity as compared to a wild type interferon-α2b polypeptide of SEQ ID NO: 24; or
the polypeptide comprises the amino acid sequence with at least 80% homology to SEQ ID NO: 23 and the polypeptide has a reduced immunogenicity as compared to a wild type interferon-α2b polypeptide of SEQ ID NO: 23.

126. The modified interferon-α2 polypeptide of claim 109, wherein
the polypeptide comprises the amino acid sequence with at least 80% homology to SEQ ID NO: 12 and has a relative antiviral activity of between 5% and 95% as compared to a wild type interferon-α2b polypeptide of SEQ ID NO: 12;
the polypeptide comprises the amino acid sequence with at least 80% homology to SEQ ID NO: 10 and has a relative antiviral activity of between 5% and 95% as compared to a wild type interferon-α2b polypeptide of SEQ ID NO: 10;
the polypeptide comprises the amino acid sequence with at least 80% homology to SEQ ID NO: 22 and has a relative antiviral activity of between 5% and 95% as compared to a wild type interferon-α2b polypeptide of SEQ ID NO: 22;
the polypeptide comprises the amino acid sequence with at least 80% homology to SEQ ID NO: 21 and has a relative antiviral activity of between 5% and 95% as compared to a wild type interferon-α2b polypeptide of SEQ ID NO: 21;
the polypeptide comprises the amino acid sequence with at least 80% homology to SEQ ID NO: 24 and has a relative antiviral activity of between 5% and 95% as compared to a wild type interferon-α2b polypeptide of SEQ ID NO: 24; or
the polypeptide comprises the amino acid sequence with at least 80% homology to SEQ ID NO: 23 and has a relative antiviral activity of between 5% and 95% as compared to a wild type interferon-α2b polypeptide of SEQ ID NO: 23.

127. The polypeptide of claim 109 at a therapeutically effective amount and in a pharmaceutical composition configured for administration to a subject.

128. A method of treating one or more diseases in a subject, comprising administering to the subject one or more of the modified interferon-α2 polypeptides of claim 109, wherein said disease is selected from the group consisting of melanomas (including malignant melanoma), chronic hepatitis C (including in patients with compensated liver disease), acute and chronic hepatitis B, acute and chronic non-A, non-B hepatitis, Kaposi's sarcoma (including AIDS-related Kaposi's sarcoma), multiple sclerosis, genital warts, leukemia (including Hairy cell leukemia), lymphomas (including follicular lymphoma), condylomata acumiate, SARS-CoV-2 infection, ZIKV infection, CHIKV infection, and influenza A infection.

129. A method of isolating the polypeptide of claim 109 comprising:
contacting a sample with an antibody using immunoaffinity chromatography, wherein said antibody comprises an anti-nonglycosylated rhIFN-α2b mAb CA5E6 antibody, an anti-hGM-CSF monoclonal antibody, or both.
